(19) United States
(12) Patent Application Publication
(10) Pub. No.: US 2016/0115136 A1
(43) Pub. Date: Apr. 28, 2016

(54) COMPOUNDS, COMPOSITIONS COMPRSING SAME, AND METHODS RELATED THERETO

(71) Applicant: UNIVERSITY OF IOWA RESEARCH FOUNDATION, Iowa City, IA (US)

(72) Inventors: Michael Ashley Spies, Iowa City, IA (US); Katie Lynn Whalen, Coralville, IA (US)

(21) Appl. No.: 14/776,508

(22) PCT Filed: Mar. 13, 2014
(86) PCT No.: PCT/US14/26513

§ 371 Date: Sep. 14, 2015

Related U.S. Application Data
(60) Provisional application No. 61/779,727, filed on Mar. 13, 2013.

(51) Int. Cl. C07D 235/28; A61K 31/5383; C07D 403/12; C07D 403/04; A61K 31/5377; C07D 491/056; C07D 498/04; A61K 31/4184

(52) U.S. Cl. CPC C07D 235/28; C07D 498/04; A61K 31/5383; A61K 31/4184; C07D 403/04; A61K 31/5377; C07D 491/056; C07D 403/12

(57) ABSTRACT

Disclosed herein are compounds, such as benzimidazole derivatives, and composition, such as pharmaceutical compositions, and methods related thereto for treating or preventing microbial infections. This abstract is intended as a scanning tool for purposes of searching in the particular art and is not intended to be limiting of the present invention.

### CROSS-REFERENCE TO RELATED APPLICATIONS

This application claims the benefit of U.S. Provisional Application No. 61/779,727, filed on Mar. 13, 2013, which is incorporated herein by reference in its entirety.

### STATEMENT REGARDING FEDERALLY SPONSORED RESEARCH

This invention was made with government support under grant GM097373 awarded by the National Institute of Health (NIH). The United States government has certain rights in the invention.

### BACKGROUND

D-amino acids play a number of vital roles in the physiology of microorganisms, making their production a potentially attractive avenue for antimicrobial drug development. The D-stereoisomer is an exotic form of amino acids (which are usually in the mirror image, L-form), which are incorporated into the peptidoglycan layer of bacterial cell walls in order to make them resistant to degradation. Glutamate racemase (GR) is part of an extended family of “cofactor-independent” racemases and epimerases, which are able to convert the normal L-amino acid to a D-amino acid. Without glutamate racemase, D-glutamate cannot be acquired and peptidoglycan synthesis is shut down. Several knockout studies have confirmed the absence of glutamate racemase as being a lethal mutation for bacteria. Thus, inhibition of this enzyme has great promise as a therapeutic route for fighting bacterial infections. Additionally, no currently available antibiotics target this particular enzyme, reducing the likelihood of immediate antibiotic resistance against these therapeutics.

However, the discovery of competitive, reversible (i.e. not mechanism based inhibitors, which are often too reactive to be good drug leads) for glutamate racemase, has been especially challenging. In fact, a recent Nature paper by a team at AstraZeneca plc., (Lundqvist et al. Nature 2007, 447 (7146), 817-22.) which reported novel uncompetitive inhibitors for glutamate racemase, indicated that a screening of their entire library of nearly 400,000 compounds did not lead to the identification of a single competitive inhibitor. Furthermore, new antimicrobial, such as antibacterial, drugs have been introduced to the market since the 1960. Since then drug- and multidrug-resistant strains of bacteria have developed. Accordingly, there is an increasing need for the development of new antimicrobial compounds.

Described herein are compounds, compositions, and methods related to antimicrobial activities.

### SUMMARY

In accordance with the purpose(s) of the invention, as embodied and broadly described herein, the invention, in one aspect, relates to compounds, articles comprising the compounds which are useful in methods to treat or prevent microbial infections, such as bacterial infections.

Disclosed herein are compounds having the formula:

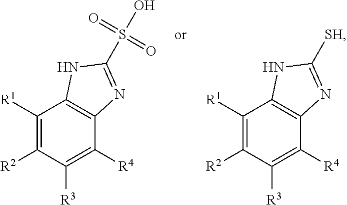

wherein R¹, R², R³, and R⁴ are independently selected from —H, halogen, —OH, —CN, —NH₂, —NO₂, —N₃, —CF₃, —C(O)O—R⁵, —S(O)ₙR⁶, —NHR⁷, —C(O)R⁸, C1-C6 alkyl, —(C1-C6 alkyl)-surface anchoring moiety, surface anchoring moiety, C2-C6 alkenyl, C2-C6 alkynyl, C1-C6 haloalkyl, C1-C6 polyhaloalkyl, C1-C6 alkoxy, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, and Cy¹; R¹ and R² are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; R² and R³ are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; R³ and R⁴ are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; wherein each n is an integer independently selected from 0, 1 and 2; wherein each R⁹, when present, is independently selected from hydrogen and C1-C8 alkyl; wherein each R¹⁰, when present, is independently selected from hydrogen and C1-C8 alkyl; wherein each R¹¹, when present, is independently selected from hydrogen and C1-C8 alkyl; wherein each R¹², when present, is independently selected from hydrogen, —OH, C1-C8 alkyl, C1-C8 hydroxyalkyl, C1-C8 monohaloalkyl, C1-C8 polyhaloalkyl, C3-C9 cycloalkyl, C2-C7 heterocycloalkyl, —(C1-C6)-Ar², and Ar²; wherein each R¹³, when present, is independently selected from hydrogen and C1-C8 alkyl; wherein each R¹⁴, when present, is independently selected from hydrogen, —OH, C1-C8 alkyl, C3-C9 cycloalkyl, C2-C7 heterocycloalkyl, C1-C8 monohaloalkyl, C1-C8 polyhaloalkyl, —(C1-C6)-Ar³, and Ar³; wherein each Ar³, when present, is independently selected from phenyl, naphthyl, and heteroaryl, and wherein each Ar³ is independently substituted with 0, 1, 2, or 3 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino; wherein each Ar², when present, is independently selected from phenyl, naphthyl, and heteroaryl, and wherein each Ar² is independently substituted with 0, 1, 2, or 3 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino; wherein each Ar¹, when present, is selected from phenyl and monocycle heteroaryl; and wherein Ar¹ is substituted with 0, 1, 2, or 3 groups independently selected from halogen, —OH, —CN, —NH₂, —NO₂, —N₃, —NHR, —C(O)R⁸, —C(O)O—R⁵, —SO₂R⁶, C1-C6 alkyl, —(C1-C6 alkyl)-surface anchoring moiety, surface anchoring moiety, C1-C6 alkoxy, C1-C6 haloalkyl, or C1-C6 polyhaloalkyl, C1-C6 alkylamino, and C1-C6 dialkylamino; wherein each Cy¹, when present, is selected from C3-C9 cycloalkyl and C3-C8 heterocycloalkyl; and wherein Cy¹ is substituted with 0, 1, 2, or 3 groups independently selected from halogen, —OH, —CN, —NH₂, —NO₂, —N₃, —NHR⁷, —C(O)R⁸, —C(O)O—R⁵, —SO₂R⁶, C1-C6 alkyl, C1-C6 alkoxy, C1-C6 haloalkyl, or C1-C6 polyhaloalkyl, C1-C6 alkylamino, and C1-C6 dialkylamino; wherein each R⁵, when present, is independently selected from —H, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety; wherein each R⁶, when present, is independently selected from —H, —OH, —NHR², C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, and a surface anchoring moiety; wherein each R⁷, when present, is independently selected from —H, —C(O)R⁸, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety; and wherein each R⁸, when present, is independently selected from —H, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹ C1-C6-surface anchoring moiety, and a surface anchoring unit; or a pharmaceutically acceptable salt, solvate, or polymorph thereof.

In one aspect, the compound is not:

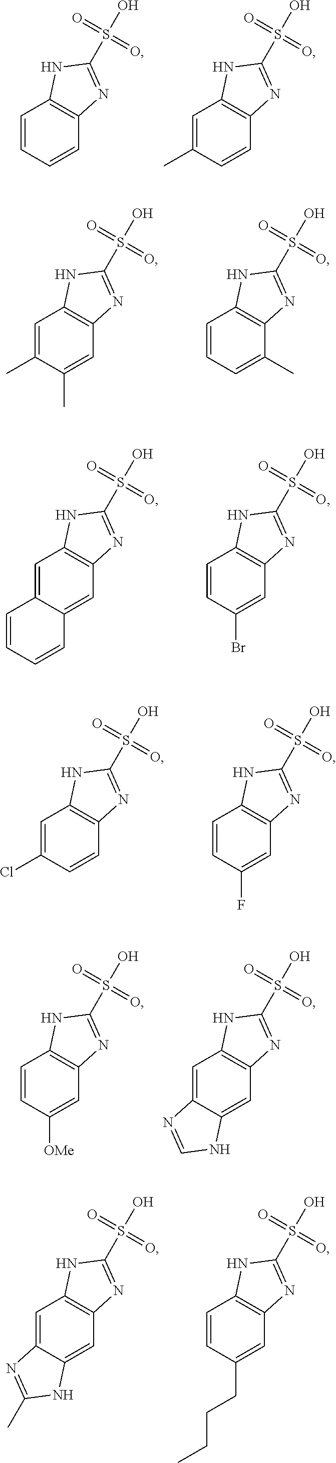

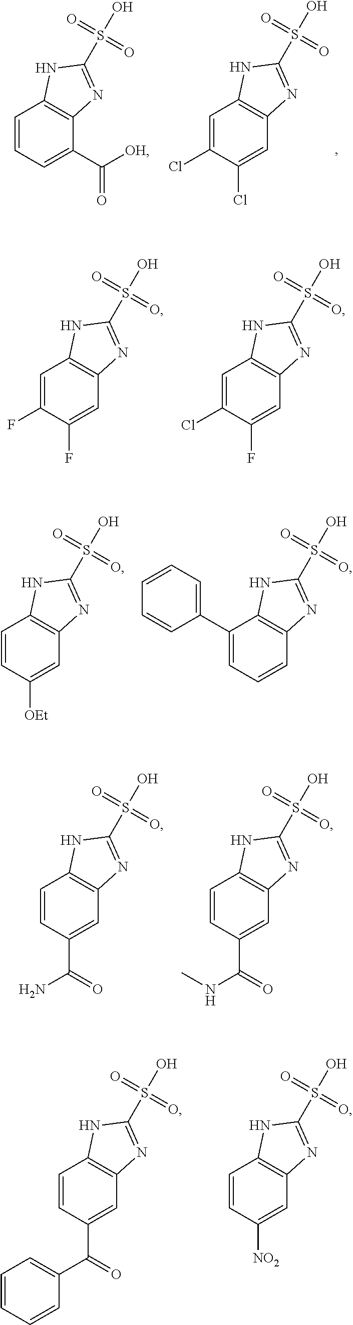

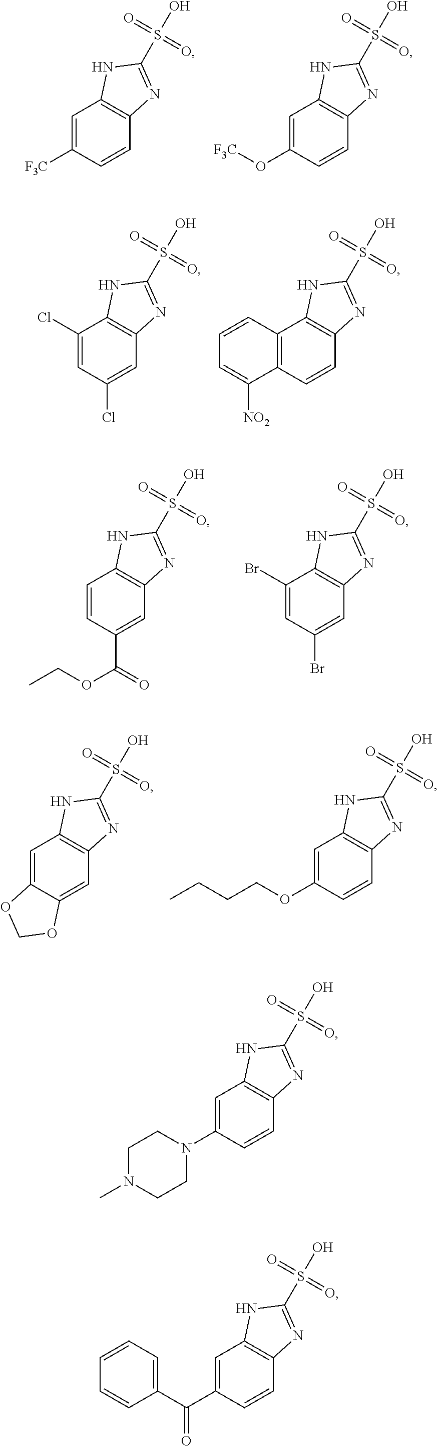

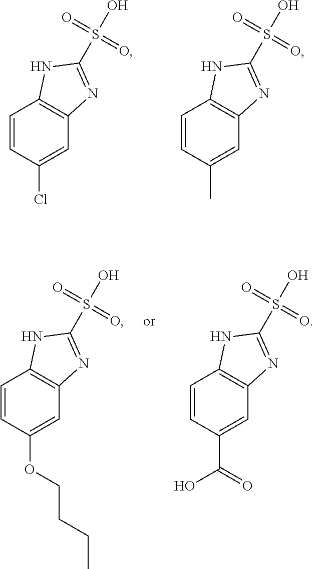 [SMILES: O=S(=O)(O)c1nc2cc(Cl)ccc2[nH]1 ; Cc1ccc2[nH]c(S(=O)(=O)O)nc2c1 ; CCCCOc1ccc2[nH]c(S(=O)(=O)O)nc2c1 ; O=C(O)c1ccc2[nH]c(S(=O)(=O)O)nc2c1]

Also disclosed herein is a pharmaceutical composition comprising a therapeutically effective amount of one or more of the disclosed compounds.

In one aspect, the pharmaceutical composition can further comprise a penicillin, a cephalosporin, a vanomycin, a bacitracin, a monobactams, a fosfomycin, a cycloserine, or a polymyxin, or a mixture thereof.

Also disclosed herein is a method comprising administering a therapeutically effective amount of one or more of the disclosed compounds to a subject.

Also disclosed herein is an article comprising at least a first surface, wherein the first surface comprises one or more of the disclosed compounds.

Also disclosed herein is a kit comprising one or more of the disclosed compounds and at least one of the following: a. at least one antibiotic agent, b. instructions for treating a disorder associated with microbial activity, c. instructions for treating an infection, d. a penicillin, e. a cephalosporin, f. a vanomycin, g. a bacitracin, h. a monobactams, i. a fosfomycin, j. a cycloserine, or k. a polymyxin.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows representative data demonstrating the potency of compound 2 against a range of GR isozymes isolated from the indicated bacterial species.

FIGS. 2A and 2B shows representative data demonstrating the binding pose for compound 2. Specifically, FIG. 2A shows the top-ranked binding pose for compound 2 bound to B. subtilis GR as predicted by docking. FIG. 2B shows the binding pose for compound 2 after 4 ns MD simulation with explicit water.

FIG. 3 shows representative data pertaining to a MD simulation on the glutamate-bound crystal structure of B. subtilis GR. Structures were obtained along the unbinding trajectory that corresponds approximately to the following states: closed, partially open, and fully open.

FIGS. 4A and 4B show the H¹ NMR of compound 4: ¹H NMR (500 MHz, DMSO) δ 7.79 (2H, q). FIG. 4B shows a closer perspective of the region of interest.

FIG. 5A-5H shows the liquid chromatography elution profiles and the mass spectrometry profiles for compound 4.

FIGS. 6A and 6B show the H¹ NMR of compound 18: ¹H NMR (500 MHz, DMSO) δ 8.06 (1H, s), 7.89 (2H, t), 7.73 (1H, t), 2.74 (2H, q), 1.35 (2H, t), 1.23 (2H, q), 0.80 (3H, t). FIG. 6B shows a close perspective of the region of interest.

FIG. 7A-7G shows the liquid chromatography elution profiles and the mass spectrometry profiles for compound 18.

FIGS. 8A and 8B show the H¹ NMR of compound 29: ¹H NMR (500 MHz, DMSO) δ 7.95 (1H, s), 7.91 (1H, d), 7.80 (1H, d), 3.63 (4H, s), 2.90 (4H, s). FIG. 8B shows a close perspective of the region of interest.

FIG. 9A-9G shows the liquid chromatography elution profiles and the mass spectrometry profiles for compound 29.

FIG. 10 shows representative data pertaining to an SDS-page analysis of purified glutamate racemases from B. subtilis (*), B. anthracis (*), and F. tularensis (**).

FIG. 11 shows the circular dichroism (CD) of purified proteins to assess protein foldness.

FIG. 12 shows representative data pertaining to the MIC₅₀ reagent volumes for a single replicate of one inhibitor. Inhibitor concentration varies along the X-axis of the plate. Two compounds could be assayed in triplicate per 96-well plate of bacteria. Blank wells contain 100 μL of phosphate-buffered saline (PBS) and 100 μL of 2× media.

FIG. 13 shows in vitro data for inhibition of glutamate racemase from Bacillus subtilis by compound 1 (6-fluoro-2-sulfo-1H-benzimidazole-4-carboxylic acid) (shown below). Data fit globally to a competitive inhibition model.

FIG. 14 shows in vitro data for inhibition of glutamate racemase isozyme 2 from Bacillus anthracis (BsRaceE2) by compound 4. Data fit globally to a competitive inhibition model. Compound 4 shows equal potency against isozymes from both species of Bacillus (subtilis and anthracis).

FIG. 15 shows in vivo data for inhibition of the microbial growth of Bacillus subtilis by treatment of compound 4. Data is fit to a dose-response curve to determine the Log IC₅₀ (Log Inhibitor Concentration at 50% Reduction of microbial growth). The MIC50 (Minimum Inhibitory Concentration required to inhibit the growth of 50% of bacteria) for compound 4 in FIG. 15 is 1,788 μM or 460 μg/mL.

FIG. 16 shows in vitro assay of D- to L-glutamate conversion by glutamate racemase from H. pylori (y-axis is enzyme activity in nmol/sec) with increasing concentrations of compound 4 (x-axis is the log of compound concentration in mM). Fitting of the data with a dose-response inhibition model gives an IC₅₀ value (concentration of compound where 50% enzymes are inhibited) of ˜100 μM.

FIG. 17 shows in vivo data for compound 4 for bacterial growth (square) as well as bacterial cell lysis (circle) as measured by optical density and luminescence, respectively. The EC₅₀ (effective concentration required to induce a 50% effect) value of cell lysis is within the same range as the MIC₅₀ value for bacterial growth inhibition against B. subtilis, 2 mM.

FIG. 18A-F show representative data pertaining to the in vitro inhibition data used to acquire Kᵢ values for compounds 4 (18A), 15 (18B), 18 (18C), 24 (18D), 26 (18E), and 29 (18F).

FIG. 19A-C show representative data pertaining to colloidal aggregate testing for compounds 4, 18, and 29.

FIG. 20 shows representative data comparing the MIC₅₀ curves for compounds 2, 4, and 24 against B. subtilis.

FIG. 21A-C show representative data pertaining to MIC₅₀ curves for compounds 4 (17A), 18 (17B), and 29 (17C) comparing species specificity between E. coli (), B. subtilis (), and S. aureus (▴).

FIG. 22 shows a representative illustration of the treatment of bacterial cells in the Cyto Tox-Glo assay.

FIGS. 23A and 23B shows representative data pertaining to a cytotoxicity assay that monitors cell wall lysis with a luminescent readout.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. DEFINITIONS

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” refers to the target of administration, e.g. an animal. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, fish, bird, or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more muscle disorders prior to the administering step. In some aspects of the disclosed method, the subject has been diagnosed with a need for promoting muscle health prior to the administering step. In some aspects of the disclosed method, the subject has been diagnosed with a need for promoting muscle health prior, promote normal muscle function, and/or promote healthy aging muscles to the administering step.

As used herein, the term “treatment” refers to the medical management of a patient with the intent to cure, ameliorate, stabilize, or prevent a disease, pathological condition, or disorder. This term includes the use for aesthetic and self-improvement purposes, for example, such uses include, but are not limited to, the administration of the disclosed compound in neutraceuticals, medicinal food, energy bar, energy drink, sports drink, protein bar, tea, coffee, milk, milk products, cereal, oatmeal, infant formulas, supplements (such as multivitamins). This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, fish, bird, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a microbial infection” means having been subjected to a physical examination by a person of skill, for example, a physician, and found to have a condition that can be diagnosed or treated by a compound or composition that can treat or prevent a microbial infection. As a further example, “diagnosed with a need for treating or preventing a microbial infection” refers to having been subjected to a physical examination by a person of skill, for example, a physician, and found to have a condition characterized by muscle atrophy or other disease wherein treating or preventing a microbial infection would be beneficial to the subject. Such a diagnosis can be in reference to a disorder, such as cancer or duodenal ulcers, and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to a microbial infection) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “EC₅₀,” is intended to refer to the concentration or dose of a substance (e.g., a compound or a drug) that is required for 50% enhancement or activation of a biological process, or component of a process, including a protein, subunit, organelle, ribonucleoprotein, etc. EC₅₀ also refers to the concentration or dose of a substance that is required for 50% enhancement or activation in vivo, as further defined elsewhere herein. Alternatively, EC₅₀ can refer to the concentration or dose of compound that provokes a response halfway between the baseline and maximum response. The response can be measured in an in vitro or in vivo system as is convenient and appropriate for the biological response of interest. For example, the response can be measured in vitro using cultured muscle cells or in an ex vivo organ culture system with isolated muscle fibers. Alternatively, the response can be measured in vivo using an appropriate research model such as rodent, including mice and rats. The mouse or rat can be an inbred strain with phenotypic characteristics of interest such as obesity or diabetes. As appropriate, the response can be measured in a transgenic or knockout mouse or rat wherein the a gene or genes has been introduced or knocked-out, as appropriate, to replicate a disease process.

As used herein, “IC₅₀,” is intended to refer to the concentration or dose of a substance (e.g., a compound or a drug) that is required for 50% inhibition or diminution of a biological process, or component of a process, including a protein, subunit, organelle, ribonucleoprotein, etc. IC₅₀ also refers to the concentration or dose of a substance that is required for 50% inhibition or diminution in vivo, as further defined elsewhere herein. Alternatively, IC₅₀ also refers to the half maximal (50%) inhibitory concentration (IC) or inhibitory dose of a substance. The response can be measured in an in vitro or in vivo system as is convenient and appropriate for the biological response of interest. For example, the response can be measured in vitro using cultured muscle cells or in an ex vivo organ culture system with isolated muscle fibers. Alternatively, the response can be measured in vivo using an appropriate research model such as rodent, including mice and rats. The mouse or rat can be an inbred strain with phenotypic characteristics of interest such as obesity or diabetes. As appropriate, the response can be measured in a transgenic or knockout mouse or rat wherein a gene or genes has been introduced or knocked-out, as appropriate, to replicate a disease process.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹”, “A²”, “A³”, and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A², where A¹ and A² can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes azetidine, dioxane, furan, imidazole, isothiazole, isoxazole, morpholine, oxazole, oxazole, including, 1,2,3-oxadiazole, 1,2,5-oxadiazole and 1,3,4-oxadiazole, piperazine, piperidine, pyrazine, pyrazole, pyridazine, pyridine, pyrimidine, pyrrole, pyrrolidine, tetrahydrofuran, tetrahydropyran, tetrazine, including 1,2,4,5-tetrazine, tetrazole, including 1,2,3,4-tetrazole and 1,2,4,5-tetrazole, thiadiazole, including, 1,2,3-thiadiazole, 1,2,5-thiadiazole, and 1,3,4-thiadiazole, thiazole, thiophene, triazine, including 1,3,5-triazine and 1,2,4-triazine, triazole, including, 1,2,3-triazole, 1,3,4-triazole, and the like.

The term “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O(CH₂)₀₋₄C(O)OR^{∘}; (CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘})₂; —OP(O)(OR^{∘})₂; —SiR^{∘}₃; —(C₁₋₄ straight or branched)alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6-membered heteroaryl ring), or a 5-6 saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12 saturated, partially unsaturated, or aryl mono or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(C)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R^{*}₂))₂₋₃O, or —S(C(R^{*}₂))₂₋₃S, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—. wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

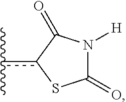

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

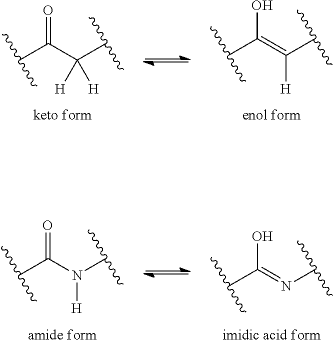 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

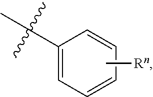

which is understood to be equivalent to a formula:

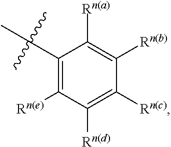

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

The compounds disclosed herein are antimicrobial compounds. In one aspect, the compounds are antibacterial. In another aspect, the compounds inhibit glutamate racemase in bacteria. In one aspect, the bacteria can be Bacillus anthracis, Staphylococcus aureus, Bacillus subtilis, or Helicobacter pylori, Escherichia coli, or a mixture thereof. In another aspect, the bacteria can be any species of bacteria, which utilizes peptidoglycan as protection from osmotic pressure.

In one aspect, the compounds disclosed herein can treat or prevent microbial infections. In another aspect, the compounds disclosed herein can treat or prevent bacterial infections. In yet another aspect, the compounds disclosed herein can treat or prevent bacterial infections from Bacillus anthracis, Staphylococcus aureus, Bacillus subtilis, or Helicobacter pylori, Escherichia coli, or a mixture thereof. For example, the compounds here can treat or prevent a bacterial infection from Staphylococcus aureus. In another example, the compounds here can treat or prevent a bacterial infection from Escherichia coli.

In one aspect, the compounds disclosed herein have an inhibitory constant (Kᵢ) of less than 20 μM, 15 μM, 10 μM, 7.5 μM, 5 μM, 2.5 μM, 1.5 μM, 1.0 μM, 0.5, μM, 0.3 μM or 0.1 μM. In another aspect, the compounds disclosed herein have an inhibitory constant (Kᵢ) of less than 5 μM, 2.5 μM, 1.5 μM, 1.0 μM, 0.5, μM, 0.3 μM or 0.1 μM. In yet another aspect, the compounds disclosed herein have an inhibitory constant (Kᵢ) of less than 2.5 μM. In yet another aspect, the compounds disclosed herein have an inhibitory constant (Kᵢ) of less than 1.0 μM. The inhibitory constant (Kᵢ) is determined by the inhibitory concentration at which microbial growth 50% of enzymes are bound to the inhibitor. Thus, Kᵢ describes the affinity for a small molecule to bind an enzyme Inhibitory constants are determined using enzyme that was purified using recombinant DNA in-house. Protocols for enzymatic assays as well as enzyme expression and purification can be found in Whalen et al. Molecular Informatics, 2011, 30, 459-471.

In one aspect, the compounds disclosed herein are negative to the formation of a colloidal aggregate. Colloidal aggregates can produce false positive results by inhibiting the enzyme target in a non-drug-like fashion. Compounds that form colloidal aggregates cannot be advanced into later stages of drug development due to their dangerous interactions with proteins of all types.

In one aspect, the compound can have the formula:

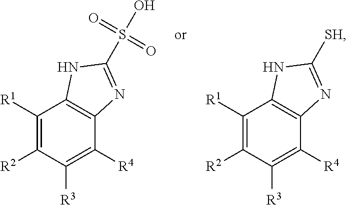

wherein R¹, R², R³, and R⁴ are independently selected from —H, halogen, —OH, —CN, —NH₂, —NO₂, —N₃, —CF₃, —C(O)O—R⁵, —S(O)ₙR⁶, —NHR⁷, —C(O)R⁸, C1-C6 alkyl, —(C1-C6 alkyl)-surface anchoring moiety, surface anchoring moiety, C2-C6 alkenyl, C2-C6 alkynyl, C1-C6 haloalkyl, C1-C6 polyhaloalkyl, C1-C6 alkoxy, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, and Cy¹; R¹ and R² are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; R² and R³ are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; R³ and R⁴ are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; wherein each n is an integer independently selected from 0, 1 and 2; wherein each R⁹, when present, is independently selected from hydrogen and C1-C8 alkyl; wherein each R¹⁰, when present, is independently selected from hydrogen and C1-C8 alkyl; wherein each R¹¹, when present, is independently selected from hydrogen and C1-C8 alkyl; wherein each R¹², when present, is independently selected from hydrogen, C1-C8 alkyl, C1-C8 hydroxyalkyl, C1-C8 monohaloalkyl, C1-C8 polyhaloalkyl, C3-C9 cycloalkyl, C2-C7 heterocycloalkyl, —(C1-C6)-Ar², and Ar²; wherein each R¹³, when present, is independently selected from hydrogen and C1-C8 alkyl; wherein each R¹⁴, when present, is independently selected from hydrogen, C1-C8 alkyl, C3-C9 cycloalkyl, C2-C7 heterocycloalkyl, C1-C8 monohaloalkyl, C1-C8 polyhaloalkyl, —(C1-C6)-Ar³, and Ar³; wherein each Ar³, when present, is independently selected from phenyl, naphthyl, and heteroaryl, and wherein each Ar³ is independently substituted with 0, 1, 2, or 3 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino; wherein each Ar², when present, is independently selected from phenyl, naphthyl, and heteroaryl, and wherein each Ar² is independently substituted with 0, 1, 2, or 3 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino; wherein each Ar¹, when present, is selected from phenyl and monocycle heteroaryl; and wherein Ar¹ is substituted with 0, 1, 2, or 3 groups independently selected from halogen, —OH, —CN, —NH₂, —NO₂, —N₃, —NHR⁷, —C(O)R⁸, —C(O)O—R⁵, —SO₂R⁶, C1-C6 alkyl, —(C1-C6 alkyl)-surface anchoring moiety, surface anchoring moiety, C1-C6 alkoxy, C1-C6 haloalkyl, or C1-C6 polyhaloalkyl, C1-C6 alkylamino, and C1-C6 dialkylamino; wherein each Cy¹, when present, is selected from C3-C9 cycloalkyl and C3-C8 heterocycloalkyl; and wherein Cy¹ is substituted with 0, 1, 2, or 3 groups independently selected from halogen, —OH, —CN, —NH₂, —NO₂, —N₃, —NHR⁷, —C(O)R⁸, —C(O)O—R⁵, —SO₂R⁶, C1-C6 alkyl, C1-C6 alkoxy, C1-C6 haloalkyl, or C1-C6 polyhaloalkyl, C1-C6 alkylamino, and C1-C6 dialkylamino; wherein each R⁵, when present, is independently selected from —H, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety; wherein each R⁶, when present, is independently selected from —H, —NHR⁷, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁶, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, and a surface anchoring moiety; wherein each R⁷, when present, is independently selected from —H, —C(O)R⁸, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety; and wherein each R⁸, when present, is independently selected from H, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁶, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, —Cy¹ C1-C6-surface anchoring moiety, and a surface anchoring unit; or a pharmaceutically acceptable salt, solvate, or polymorph thereof.

In one aspect, the compound has the formula:

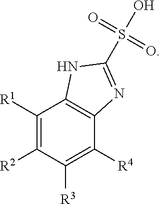

In one aspect, the compound has the formula:

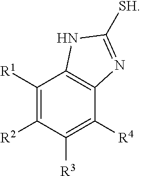

In one aspect, the compound has the formula:

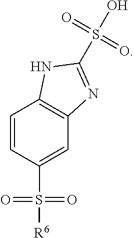

In one aspect, R⁶ can be —NHR⁷, the compound would then have the formula:

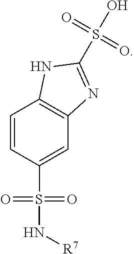

In one aspect, the compound has the formula:

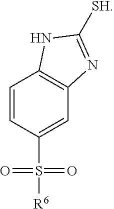

In one aspect, R⁶ can be —NHR⁷, the compound would then have the formula:

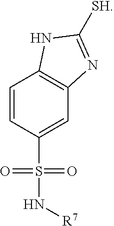

In one aspect, the compound has the formula:

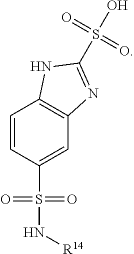

In one aspect, the compound has the formula:

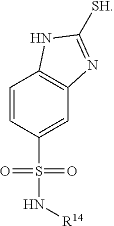

In one aspect, the compound has the formula:

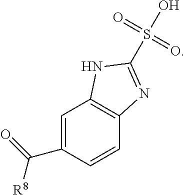

In one aspect, the compound has the formula:

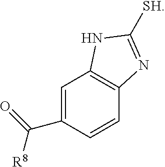

In one aspect, the compound has the formula:

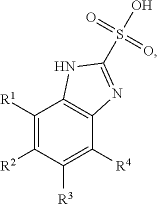

wherein R¹ and R² are covalently bonded together to form a C3-C10 cycloalkyl or C2-C7 heterocycloalkyl. For example, R¹ and R² can be covalently bonded together to form a C3-C10 cycloalkyl, such as, for example, C6, C8 or C10 cycloalkyl. In one aspect, the C3-C10 cycloalkyl, such as, for example, C6, C8 or C10 cycloalkyl, can be substituted with 0, 1, 2, or 3 groups independently selected from halogen, —NH₂, —OH, ═O, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹. For example, the C3-C10 cycloalkyl, such as, for example, C6, C8 or C10 cycloalkyl, can be substituted with 2 groups, for example, two ═O groups. In another example, R¹ and R² can be covalently bonded together to form a C2-C7 heterocycloalkyl, such as, for example, C4 or C5 heterocycloalkyl. In one example, the C2-C7 heterocycloalkyl, such as, for example, C4 or C5 heterocycloalkyl, can be substituted with 0, 1, 2, 3, or 4 groups independently selected from halogen, —NH₂, —OH, —CN, ═O, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹. In another example, the C2-C7 heterocycloalkyl, such as, for example, C4 or C5 heterocycloalkyl, can be substituted with 0 groups. In yet another example, the C2-C7 heterocycloalkyl, such as, for example, C4 or C5 heterocycloalkyl, can be substituted with 4 groups independently selected from halogen, —NH₂, —OH, ═O, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹. The C2-C7 heterocycloalkyl, such as, for example, C4 or C5 heterocycloalkyl, for example, can be substituted with 1, 2, 3, or 4 groups independently selected from halogen, ═O, C1-C6 alkyl, C1-C6 haloalkyl, C1-C6 polyhaloalkyl, C1-C6 alkoxy, Ar¹, and Cy¹.

In one aspect, the compound has the formula:

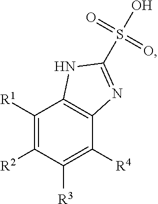

wherein R² and R³ are covalently bonded together to form a C3-C10 cycloalkyl or C2-C7 heterocycloalkyl. For example, R² and R³ can be covalently bonded together to form a C3-C10 cycloalkyl, such as, for example, C6, C8 or C10 cycloalkyl. In one aspect, the C3-C10 cycloalkyl, such as, for example, C6, C8 or C10 cycloalkyl, can be substituted with 0, 1, 2, or 3 groups independently selected from halogen, —NH₂, —OH, ═O, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹. For example, the C3-C10 cycloalkyl, such as, for example, C6, C8 or C10 cycloalkyl, can be substituted with 2 groups, for example, two ═O groups. In another example, R² and R³ can be covalently bonded together to form a C2-C7 heterocycloalkyl, such as, for example, C4 or C5 heterocycloalkyl. In one example, the C2-C7 heterocycloalkyl, such as, for example, C4 or C5 heterocycloalkyl, can be substituted with 0, 1, 2, 3, or 4 groups independently selected from halogen, —NH₂, —OH, —CN, ═O, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹. In another example, the C2-C7 heterocycloalkyl, such as, for example, C4 or C5 heterocycloalkyl, can be substituted with 0 groups. In yet another example, the C2-C7 heterocycloalkyl, such as, for example, C4 or C5 heterocycloalkyl, can be substituted with 4 groups independently selected from halogen, —NH₂, —OH, ═O, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹. The C2-C7 heterocycloalkyl, such as, for example, C4 or C5 heterocycloalkyl, for example, can be substituted with 1, 2, 3, or 4 groups independently selected from halogen, ═O, C1-C6 alkyl, C1-C6 haloalkyl, C1-C6 polyhaloalkyl, C1-C6 alkoxy, Ar¹, and Cy¹.

In one aspect, the compound has the formula:

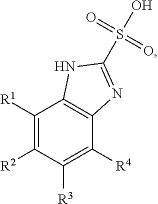

wherein R³ and R⁴ are covalently bonded together to form a C3-C10 cycloalkyl or C2-C7 heterocycloalkyl. For example, R³ and R⁴ can be covalently bonded together to form a C3-C10 cycloalkyl, such as, for example, C6, C8 or C10 cycloalkyl. In one aspect, the C3-C10 cycloalkyl, such as, for example, C6, C8 or C10 cycloalkyl, can be substituted with 0, 1, 2, or 3 groups independently selected from halogen, —NH₂, —OH, ═O, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹. For example, the C3-C10 cycloalkyl, such as, for example, C6, C8 or C10 cycloalkyl, can be substituted with 2 groups, for example, two ═O groups. In another example, R³ and R⁴ can be covalently bonded together to form a C2-C7 heterocycloalkyl, such as, for example, C4 or C5 heterocycloalkyl. In one example, the C2-C7 heterocycloalkyl, such as, for example, C4 or C5 heterocycloalkyl, can be substituted with 0, 1, 2, 3, or 4 groups independently selected from halogen, —NH₂, —OH, —CN, ═O, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹. In another example, the C2-C7 heterocycloalkyl, such as, for example, C4 or C5 heterocycloalkyl, can be substituted with 0 groups. In yet another example, the C2-C7 heterocycloalkyl, such as, for example, C4 or C5 heterocycloalkyl, can be substituted with 4 groups independently selected from halogen, —NH₂, —OH, ═O, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹. The C2-C7 heterocycloalkyl, such as, for example, C4 or C5 heterocycloalkyl, for example, can be substituted with 1, 2, 3, or 4 groups independently selected from halogen, ═O, C1-C6 alkyl, C1-C6 haloalkyl, C1-C6 polyhaloalkyl, C1-C6 alkoxy, Ar¹, and Cy¹.

In one aspect, the compound is not:

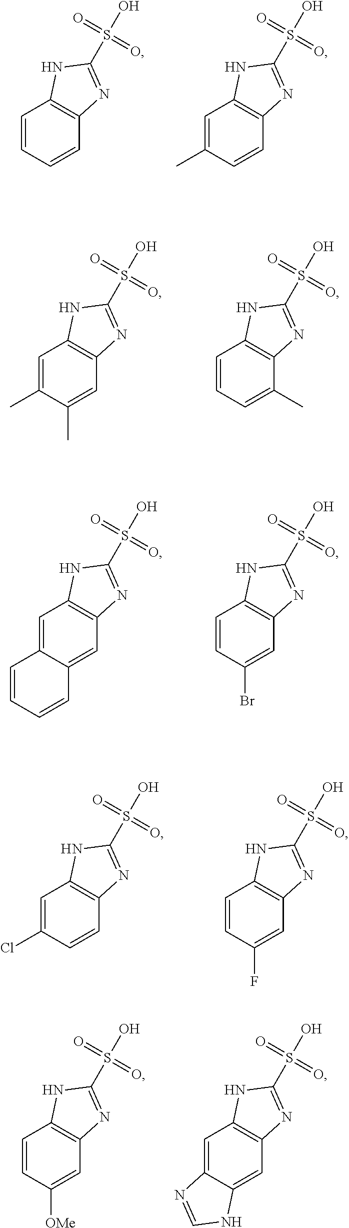

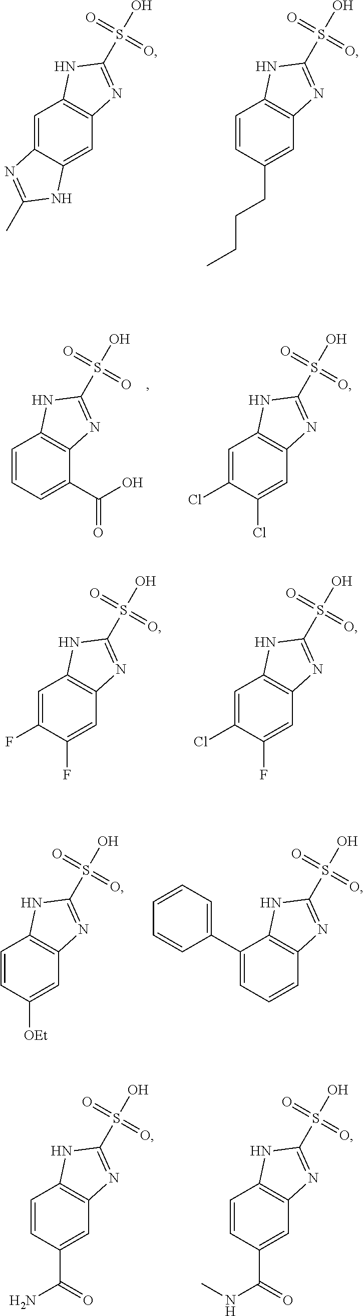

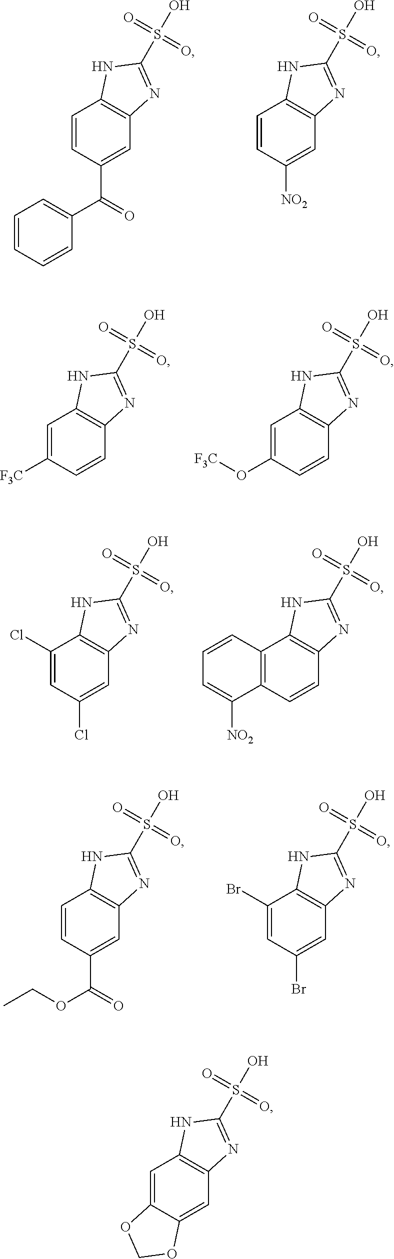

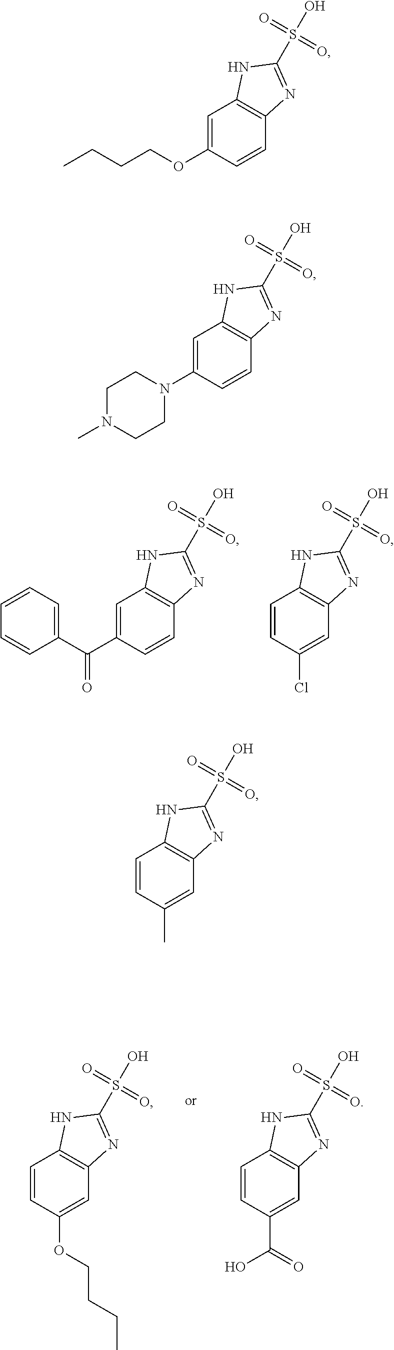

In one aspect, the compound is not:

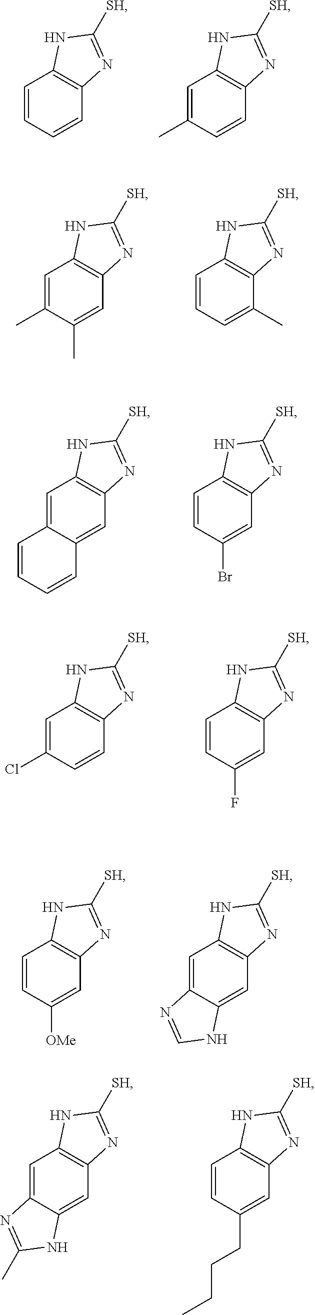

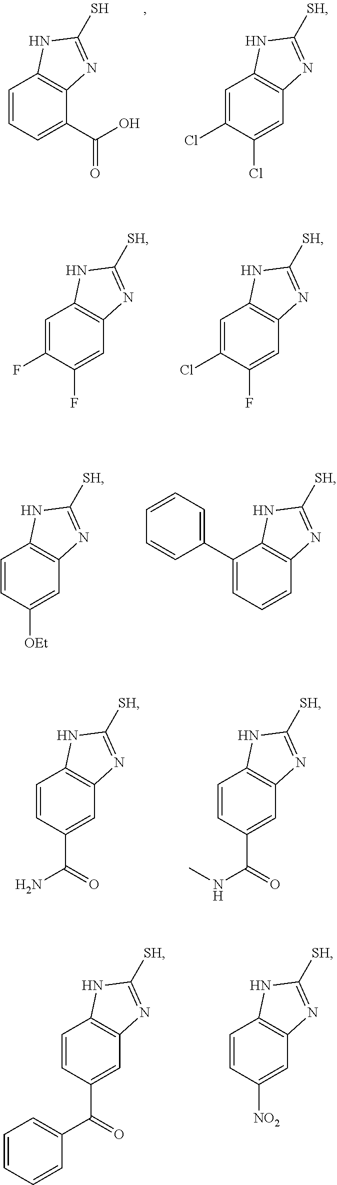

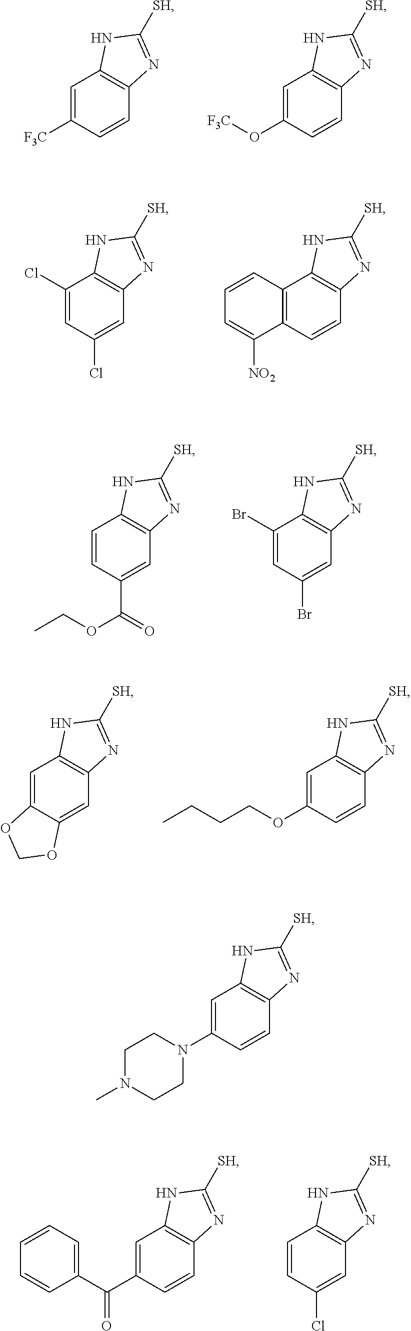

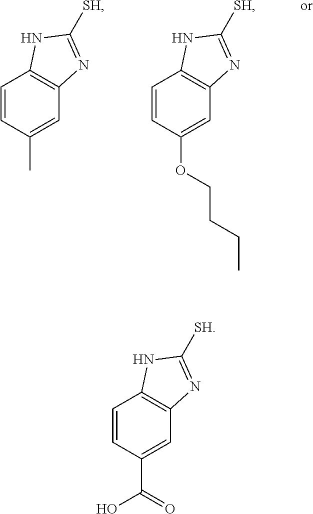

1. R¹, R², R³, and R⁴

In one aspect, at least one of R¹, R², R³, and R⁴ is selected from halogen, —OH, —C(O)O—R⁵, or —C(O)R⁸. In another aspect, at least one of R¹, R², R³, and R⁴ is —C(O)O—R⁵. In yet another aspect, at least one of R¹, R², R³, and R⁴ is —C(O)R⁸. In yet another aspect, at least one of R¹, R², R³, and R⁴ is —OH. In yet another aspect, at least one of R¹, R², R³, and R⁴ is a halogen. In yet another aspect, at least one of R¹, R², R³, and R⁴ is selected from halogen, —OH, —C(O)O—R⁵, or —C(O)R⁸ and at least one of R¹, R², R³, and R⁴ is a halogen. In yet another aspect, R⁴ is selected from halogen, —OH, —C(O)O—R⁵, or —C(O)R⁸ and R² is a halogen. In yet another aspect, at least one of R¹, R², R³, and R⁴ is C1-C6 alkoxy. In yet another aspect, at least one of R¹, R², R³, and R⁴ is C1-C6 alkyl. In yet another aspect, at least one of R¹, R², R³, and R⁴ is —(C1-C6 alkyl)-surface anchoring moiety or a surface anchoring moiety. In yet another aspect, at least one of R¹, R², R³, and R⁴ is —H. In yet another aspect, at least two of R¹, R², R³, and R⁴ are —H. In yet another aspect, at least three of R¹, R², R³, and R⁴ are —H. In yet another aspect, at least one of R¹, R², R³, and R⁴ is C1-C6 alkylamino or C1-C6 dialkylamino. In yet another aspect, at least one of R¹, R², R³, and R⁴ is —NO₂. In yet another aspect, at least one of R¹, R², R³, and R⁴ is —CN. In yet another aspect, at least one of R¹, R², R³, and R⁴ is C1-C6 haloalkyl. In yet another aspect, at least one of R¹, R², R³, and R⁴ is —CF₃. In yet another aspect, at least one of R¹, R², R³, and R⁴ is Cy¹. In yet another aspect, at least one of R¹, R², R³, and R⁴ is Ar¹.

In yet another aspect, at least one of R¹, R², R³, and R⁴ is S(O)ₙR⁶, wherein n is 1 or 2. In yet another aspect, at least one of R¹, R², R³, and R⁴ is S(O)ₙR⁶, wherein n is 1. In yet another aspect, at least one of R¹, R², R³, and R⁴ is S(O)ₙR⁶, wherein n is 2. For example, R² can be S(O)ₙR⁶, wherein n is 2. In another example, R² can be S(O)ₙR⁶, wherein n is 2, and R¹, R³, and R⁴ can be —H. In yet another example, R³ can be S(O). R⁶, wherein n is 2. In yet another example, R³ can be S(O)ₙR⁶, wherein n is 2, and R¹, R², and R⁴ can be —H.

In yet another aspect, R² and R³ are covalently bonded together. In yet another aspect, R¹ and R² are covalently bonded together. In yet another aspect, R³ and R⁴ are covalently bonded together.

2. R⁵

In one aspect, each R⁵, when present, is independently selected from —H, C1-C6 alkyl, C1-C6 haloalkyl, C1-C6 polyhaloalkyl, C1-C6 alkylamino, C1-C6 dialkylamino, —(C1-C3 alkyl)-Ar¹, —(C1-C3 alkyl)-Cy¹, C1-C6-surface anchoring moiety, and a surface anchoring moiety. In another aspect, each R⁵, when present, is independently selected from —H, C1-C6 alkyl, —(C1-C3 alkyl)-Ar¹, —(C1-C3 alkyl)-Cy¹, C1-C6-surface anchoring moiety, and a surface anchoring moiety. In yet another aspect, each R⁵, when present, is independently selected from —H, C1-C6 alkyl, C1-C6-surface anchoring moiety, and a surface anchoring moiety. In another aspect, each R⁵, when present, is —H. In yet another aspect, each R⁵, when present, is C1-C6-surface anchoring moiety. In yet another aspect, each R⁵, when present, is a surface anchoring moiety. In yet another aspect, each R⁵, when present, is C1-C6 alkyl.

3. n

In one aspect, n can be 0. In another aspect, n can be 1. In another aspect, n can be 2. In yet another aspect, n can be 1 or 2.

4. R⁶

In one aspect, each R⁶, when present, is independently selected from —H, —OH, —NHR⁷, C1-C6 haloalkyl, —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², —NR¹¹S(O)ₙR¹², —(C1-C3 alkyl)-Ar¹, Ar¹, —(C1-C3 alkyl)-Cy¹, Cy¹, and a surface anchoring moiety. In another aspect, each R⁶, when present, is independently selected from —OH, —NHR⁷, C1-C6 haloalkyl, —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², and —NR¹¹S(O)ₙR¹². In yet another aspect, each R⁶, when present, is —NHR⁷. In yet another aspect, each R⁶, when present, is C1-C6 haloalkyl. In yet another aspect, each R⁶, when present, is C1-C6 haloalkyl. In yet another aspect, each R⁶, when present, is independently selected from —NR¹¹(C1-C6 alkyl)-(C═O)R¹², —NR¹¹(C1-C6 alkyl)-(C═O)OR¹², NR¹¹(C1-C6 alkyl)-(C═O)NR¹², NR¹¹(C1-C6 alkyl)-S(O)ₙR¹², NR¹¹(C1-C6 alkyl)-S(O)ₙNR⁹R¹⁰, —NR¹¹(C═O)R¹², —NR¹¹(C═O)OR¹², —NR¹¹(C═O)NR¹², and —NR¹¹S(O)ₙR¹².

5. R⁷

In one aspect, each R⁷, when present, is independently selected from —H, —C(O)R⁸, C1-C6 alkyl, C1-C6 haloalkyl, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety. In another aspect, each R⁷, when present, is independently selected from —H, —C(O)R⁸, C1-C6 alkyl, —(C1-C3 alkyl)-Ar¹, Ar¹, —(C1-C3 alkyl)-Cy¹, Cy¹, C1-C6-surface anchoring moiety, and a surface anchoring moiety. In yet another aspect, each R⁷, when present, is independently selected from —H, —C(O)R⁸, and C1-C6 alkyl. In yet another aspect, each R⁷, when present, is independently selected from Ar¹ and Cy¹. For example, each R⁷, when present, is Ar¹. In another example, each R⁷, when present, is Cy¹. In yet another aspect, each R⁷ is —H. In yet another aspect, each R⁷ is —C(O)R⁸. In yet another aspect, each R⁷ is a C1-C6-surface anchoring moiety. In yet another aspect, each R⁷ is a surface anchoring moiety.

6. R⁸

In one aspect, each R⁸, when present, is independently selected from —H, C1-C6 alkyl, C1-C6 haloalkyl, C1-C6 polyhaloalkyl, C1-C6 alkylamino, C1-C6 dialkylamino, —(C1-C3 alkyl)-Ar¹, —(C1-C3 alkyl)-Cy¹, C1-C6-surface anchoring moiety, and a surface anchoring moiety. In another aspect, each R⁸, when present, is independently selected from —H, C1-C6 alkyl, —(C1-C3 alkyl)-Ar¹, —(C1-C3 alkyl)-Cy¹, C1-C6-surface anchoring moiety, and a surface anchoring moiety. In yet another aspect, each R⁸, when present, is independently selected from —H, C1-C6 alkyl, C1-C6-surface anchoring moiety, and a surface anchoring moiety. In another aspect, each R⁸, when present, is —H. In yet another aspect, each R⁸, when present, is C1-C6-surface anchoring moiety. In yet another aspect, each R⁸, when present, is a surface anchoring moiety. In yet another aspect, each R⁸, when present, is C1-C6 alkyl. n yet another aspect, each R⁸, when present, is Ar¹ or Cy¹. For example, R⁸, when present, can be Ar¹.

7. R⁹

In one aspect, R⁹ is hydrogen. In another aspect, R⁹ is C1-C8 alkyl.

8. R¹⁰

In one aspect, R¹⁰ is hydrogen. In another aspect, R¹⁰ is C1-C8 alkyl.

9. R¹¹

In one aspect, R¹¹ is hydrogen. In another aspect, R¹¹ is C1-C8 alkyl.

10. R¹²

In one aspect, each R¹², when present, is independently selected from hydrogen, —OH, C1-C8 alkyl, C3-C9 cycloalkyl, C2-C7 heterocycloalkyl, —(C1-C6)-Ar², and Ar². In another aspect, each R¹², when present, is independently selected from hydrogen, —OH, and C1-C8 alkyl. In another aspect, each R¹² is hydrogen. In yet another aspect, each R¹² is —OH. In yet another aspect, each R¹² is C1-C8 alkyl. In yet another aspect, each R¹² is —(C1-C6)-Ar², or Ar².

11. R¹³

In one aspect, R¹³ is hydrogen. In another aspect, R¹³ is C1-C8 alkyl.

12. R¹⁴

In one aspect, each R¹⁴, when present, is independently selected from hydrogen, —OH, C1-C8 alkyl, C3-C9 cycloalkyl, C2-C7 heterocycloalkyl, —(C1-C6)-Ar³, and Ar³. For example, R¹⁴, when present, can be C3-C9 cycloalkyl, such as, for example, C6 cycloalkyl. In one aspect, the C3-C9 cycloalkyl, such as, for example, C6 cycloalkyl, can be substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹. For example, the C3-C9 cycloalkyl, such as, for example, C6 cycloalkyl, can be substituted with 1 group, for example, a C1-C6 alkoxy group, such as, for example, methoxy. In another example, R¹⁴, when present, can be C2-C7 heterocycloalkyl, such as, for example, C3 heterocycloalkyl. In one example, the C2-C7 heterocycloalkyl, such as, for example, C3 heterocycloalkyl, can be substituted with 0, 1, 2, 3, or 4 groups independently selected from halogen, —NH₂, —OH, —CN, ═O, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹. In another example, the C2-C7 heterocycloalkyl, such as, for example, C3 heterocycloalkyl, can be substituted with 0 groups. In yet another example, the C2-C7 heterocycloalkyl, such as, for example, C3 heterocycloalkyl, can be substituted with 4 groups independently selected from halogen, —NH₂, —OH, ═O, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹. The C2-C7 heterocycloalkyl, such as, for example, C3 heterocycloalkyl, for example, can be substituted with 1, 2, 3, or 4 groups independently selected from halogen, ═O, C1-C6 alkyl, C1-C6 haloalkyl, C1-C6 polyhaloalkyl, C1-C6 alkoxy, Ar¹, and Cy¹. In another aspect, each R¹⁴, when present, is independently selected from hydrogen, —OH, and C1-C8 alkyl. In another aspect, each R¹² is hydrogen. In yet another aspect, each R¹⁴ is —OH. In yet another aspect, each R¹⁴ is C1-C8 alkyl. In yet another aspect, each R¹⁴ is —(C1-C6)-Ar³, or Ar^{a}.

13. Ar¹

In one aspect, each Ar¹, when present, is phenyl. In another aspect, each Ar¹, when present, is monocycle heteroaryl. In yet another aspect, each Ar¹, when present, is independently substituted with 0, 1, or 2, groups independently selected from halogen, —OH, —CN, —NH₂, —NO₂, —N₃, —NHR⁷, —C(O)R⁸, —C(O)O—R⁵, —SO₂R⁶, C1-C6 alkyl, —(C1-C6 alkyl)-surface anchoring moiety, surface anchoring moiety, C1-C6 alkoxy, C1-C6 haloalkyl, or C1-C6 polyhaloalkyl, C1-C6 alkylamino, and C1-C6 dialkylamino. In yet another aspect, each Ar¹, when present, is independently substituted with 0 or 1 groups independently selected from halogen, —OH, —CN, —NH₂, —NO₂, —N₃, —NHR⁷, —C(O)R⁸, —C(O)O—R⁵, —SO₂R⁶, C1-C6 alkyl, —(C1-C6 alkyl)-surface anchoring moiety, surface anchoring moiety, C1-C6 alkoxy, C1-C6 haloalkyl, or C1-C6 polyhaloalkyl, C1-C6 alkylamino, and C1-C6 dialkylamino. In yet another aspect, each Ar¹, is substituted with 0 groups.

14. Ar²

In one aspect, each Ar², when present, is independently selected from phenyl, and heteroaryl. In another aspect, each Ar², when present, is phenyl. In yet another aspect, each Ar², when present, is naphthyl. In yet another aspect, each Ar², when present, is heteroaryl. In yet another aspect, each Ar² is independently substituted with 0, 1, or 2 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino. In yet another aspect, each Ar² is independently substituted with 0, or 1 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino. In yet another aspect, each Ar² is substituted with 0 groups.

15. Ar³

In one aspect, each Ar³, when present, is independently selected from phenyl, and heteroaryl. In another aspect, each Ar³, when present, is phenyl. In yet another aspect, each Ar², when present, is naphthyl. In yet another aspect, each Ar³, when present, is heteroaryl. In yet another aspect, each Ar³ is independently substituted with 0, 1, or 2 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino. In yet another aspect, each Ar³ is independently substituted with 0, or 1 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino. In yet another aspect, each Ar³ is substituted with 0 groups.

16. Cy¹

In one aspect, each Cy¹, when present, is C3-C9 cycloalkyl. In another aspect, each Cy¹, when present, is C3-C8 heterocycloalkyl. In yet another aspect, each Cy¹, when present, is independently substituted with 0, 1, or 2 groups independently selected from halogen, —OH, —CN, —NH₂, —NO₂, —N₃, —NHR⁷, —C(O)R⁸, —C(O)O—R⁵, —SO₂R⁶, C1-C6 alkyl, C1-C6 alkoxy, C1-C6 haloalkyl, or C1-C6 polyhaloalkyl, C1-C6 alkylamino, and C1-C6 dialkylamino. In yet another aspect, each Cy¹, when present, is independently substituted with 0, or 1 groups independently selected from halogen, —OH, —CN, —NH₂, —NO₂, —N₃, —NHR⁷, —C(O)R⁸, —C(O)O—R⁵, —SO₂R⁶, C1-C6 alkyl, C1-C6 alkoxy, C1-C6 haloalkyl, or C1-C6 polyhaloalkyl, C1-C6 alkylamino, and C1-C6 dialkylamino. In yet another aspect, each Cy¹, when present, is with 0 groups. In yet another aspect, each Cy¹, when present, is independently substituted with 4 groups independently selected from halogen, —OH, —CN, —NH₂, —NO₂, —N₃, —NHR⁷, —C(O)R⁸, —C(O)O—R⁵, —SO₂R⁶, C1-C6 alkyl, C1-C6 alkoxy, C1-C6 haloalkyl, or C1-C6 polyhaloalkyl, C1-C6 alkylamino, and C1-C6 dialkylamino. In yet another aspect, each Cy¹, when present, is independently substituted with 3 groups independently selected from halogen, —OH, —CN, —NH₂, —NO₂, —N₃, —NHR⁷, —C(O)R⁸, —C(O)O—R⁵, —SO₂R⁶, C1-C6 alkyl, C1-C6 alkoxy, C1-C6 haloalkyl, or C1-C6 polyhaloalkyl, C1-C6 alkylamino, and C1-C6 dialkylamino.

17. Surface Anchoring Moiety

A “surface anchoring moiety” is a chemical entity capable of interacting with a surface. The interaction can be either via covalent bonds or via a non-covalent interaction. In one aspect, the interaction can be via covalent bonds. Thus, the surface anchoring moiety is covalently bonded to the surface.

The surface can be any surface. For example, the surface can be the outer surface of an article. In one aspect, the article can be medical device.

The surface anchoring moiety as part of the disclosed compounds can be any molecule capable of providing adhesion or fixation to a surface. The skilled person will understand that the surface anchoring moiety will depend on the surface chemistry of the material to which the finish is to be applied. For example, textile applications can have terminal hydroxyl, carboxyl or amine groups to which the surface anchoring group can be bonded. For wood applications hydroxyl groups are present to be bonded with the surface anchoring group. In the case of glass, metal and ceramics also hydroxyl groups are present.

In one aspect, the surface anchoring moiety can comprise an epoxy moiety, urethane moiety, alkoxysilane moiety, chlorosilane moiety, alkoxytitane moiety, hydroxyl moiety, carboxyl moiety, amine moiety, hydrazide moiety, amide moiety or an aldehyde moiety or a mixture thereof. In another aspect, the surface anchoring moiety can comprise an epoxy moiety, urethane moiety, alkoxysilane moiety, chlorosilane moiety, carboxyl moiety, amine moiety, hydrazide moiety, or amide moiety or a mixture thereof. In yet another aspect, the surface anchoring moiety comprises an epoxy, hydroxyl, carboxyl or amine moiety.

If no suitable reactive groups are available on the surface for bonding with the surface anchoring group, then the compounds can be coated onto the surface using one or more coating techniques known in the art.

18. Specific Compounds

In one aspect, the compound is selected from:

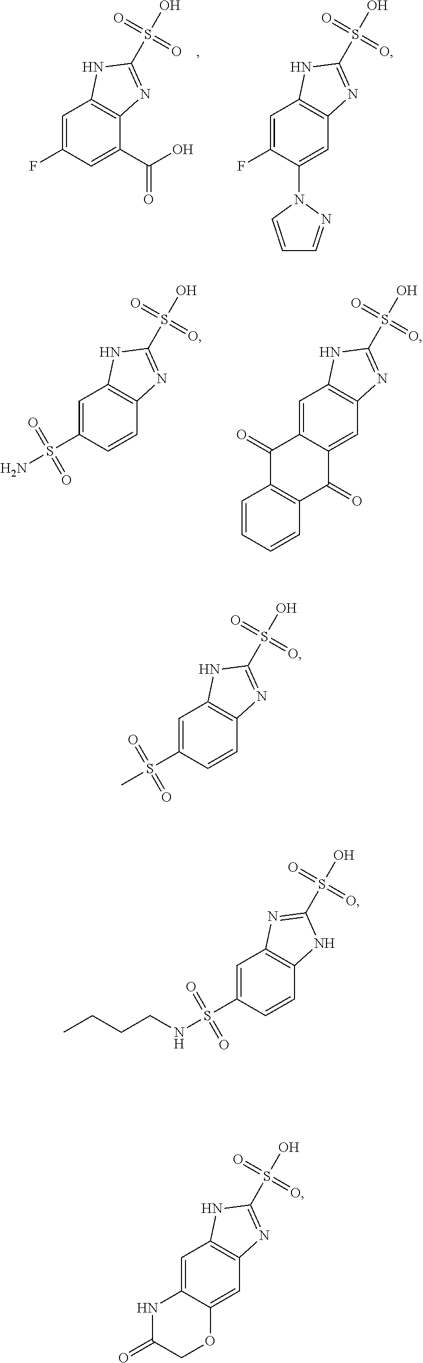

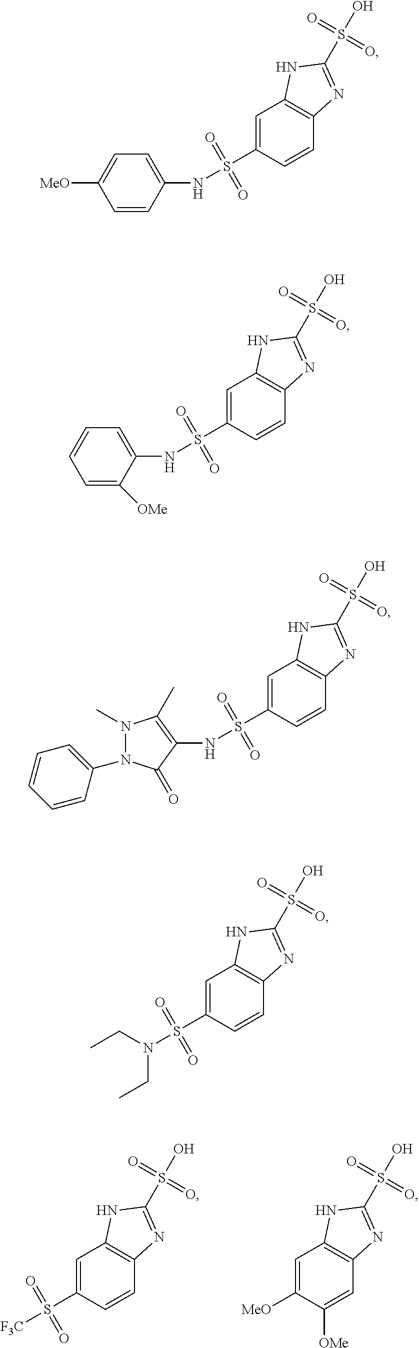 [SMILES: COc1ccc(NS(=O)(=O)c2ccc3nc(S(=O)(=O)O)[nH]c3c2)cc1 ; COc1ccccc1NS(=O)(=O)c1ccc2nc(S(=O)(=O)O)[nH]c2c1 ; Cc1c(NS(=O)(=O)c2ccc3nc(S(=O)(=O)O)[nH]c3c2)c(=O)n(-c2ccccc2)n1C ; CCN(CC)S(=O)(=O)c1ccc2nc(S(=O)(=O)O)[nH]c2c1 ; O=S(=O)(O)c1nc2ccc(S(=O)(=O)C(F)(F)F)cc2[nH]1 ; COc1cc2nc(S(=O)(=O)O)[nH]c2cc1OC]

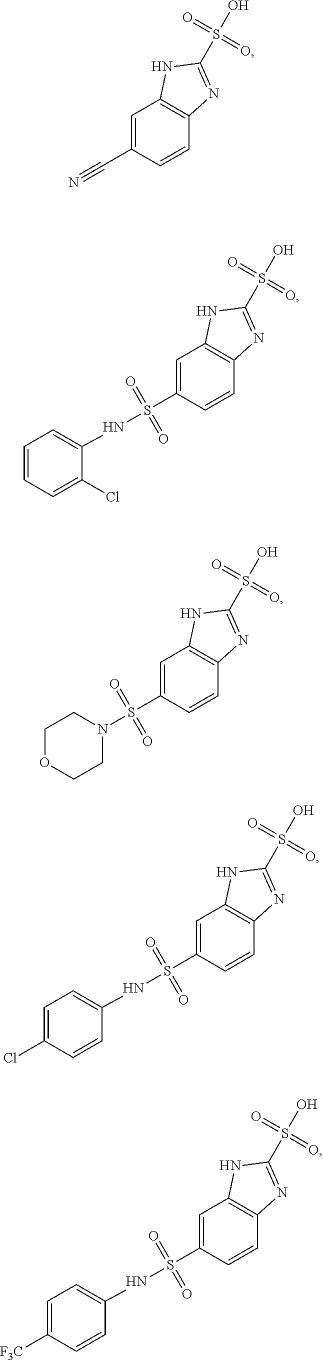 [SMILES: N#Cc1ccc2nc(S(=O)(=O)O)[nH]c2c1 ; O=S(=O)(O)c1nc2ccc(S(=O)(=O)Nc3ccccc3Cl)cc2[nH]1 ; O=S(=O)(O)c1nc2ccc(S(=O)(=O)N3CCOCC3)cc2[nH]1 ; O=S(=O)(O)c1nc2ccc(S(=O)(=O)Nc3ccc(Cl)cc3)cc2[nH]1 ; O=S(=O)(O)c1nc2ccc(S(=O)(=O)Nc3ccc(C(F)(F)F)cc3)cc2[nH]1]

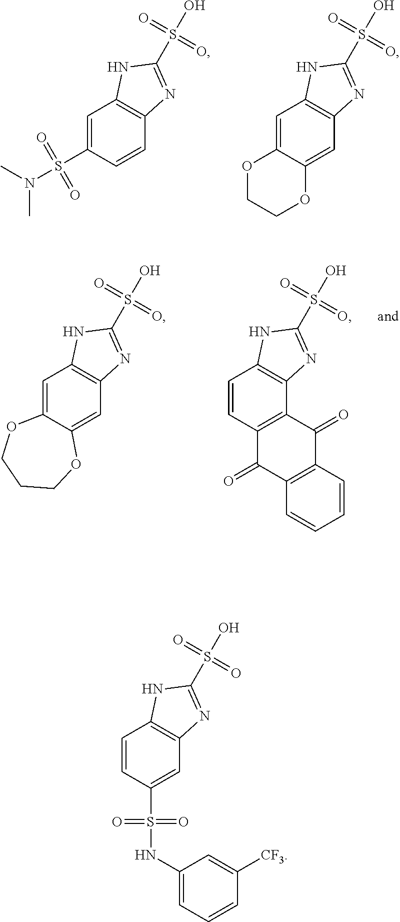 [SMILES: CN(C)S(=O)(=O)c1ccc2nc(S(=O)(=O)O)[nH]c2c1 ; O=S(=O)(O)c1nc2cc3c(cc2[nH]1)OCCO3 ; O=S(=O)(O)c1nc2cc3c(cc2[nH]1)OCCCO3 ; O=C1c2ccccc2C(=O)c2c1ccc1[nH]c(S(=O)(=O)O)nc21 ; "and" ; O=S(=O)(O)c1nc2cc(S(=O)(=O)Nc3cccc(C(F)(F)F)c3)ccc2[nH]1]

In another aspect, the compound is:

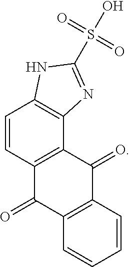 [SMILES: O=C1c2ccccc2C(=O)c2c1ccc1[nH]c(S(=O)(=O)O)nc21]

In another aspect, the compound is:

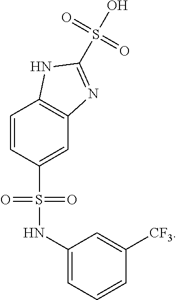 [SMILES: O=S(=O)(O)c1nc2cc(S(=O)(=O)Nc3cccc(C(F)(F)F)c3)ccc2[nH]1]

In another aspect, the compound is:

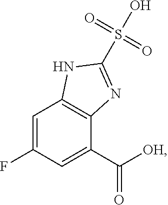 [SMILES: O=C(O)c1cc(F)cc2[nH]c(S(=O)(=O)O)nc12]

In yet another aspect, the compound is:

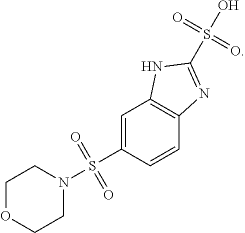 [SMILES: O=S(=O)(O)c1nc2ccc(S(=O)(=O)N3CCOCC3)cc2[nH]1]

In yet another aspect, the compound is:

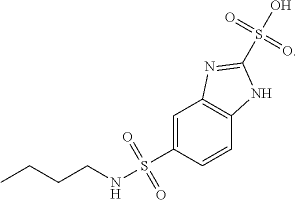 [SMILES: CCCCNS(=O)(=O)c1ccc2[nH]c(S(=O)(=O)O)nc2c1]

In yet another aspect, the compound is:

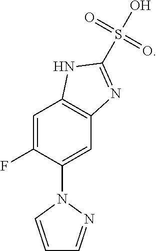 [SMILES: O=S(=O)(O)c1nc2cc(-n3cccn3)c(F)cc2[nH]1]

In yet another aspect, the compound is:

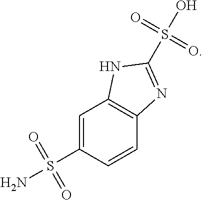 [SMILES: NS(=O)(=O)c1ccc2nc(S(=O)(=O)O)[nH]c2c1]

In yet another aspect, the compound is:

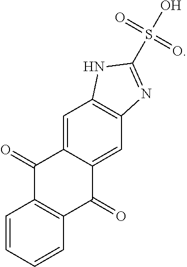 [SMILES: O=C1c2ccccc2C(=O)c2cc3[nH]c(S(=O)(=O)O)nc3cc21]

In yet another aspect, the compound is:

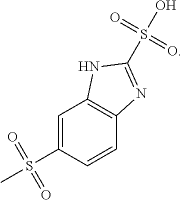 [SMILES: CS(=O)(=O)c1ccc2nc(S(=O)(=O)O)[nH]c2c1]

In yet another aspect, the compound is:

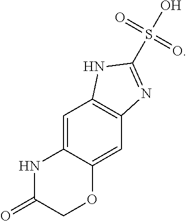 [SMILES: O=C1COc2cc3nc(S(=O)(=O)O)[nH]c3cc2N1]

In yet another aspect, the compound is:

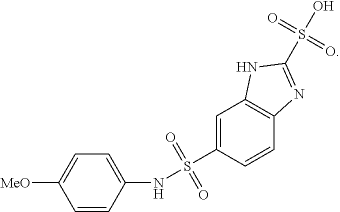 [SMILES: COc1ccc(NS(=O)(=O)c2ccc3nc(S(=O)(=O)O)[nH]c3c2)cc1]

In yet another aspect, the compound is:

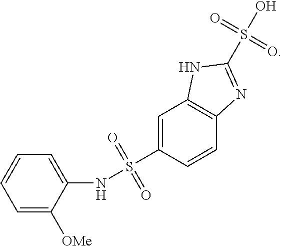 [SMILES: COc1ccccc1NS(=O)(=O)c1ccc2nc(S(=O)(=O)O)[nH]c2c1]

In yet another aspect, the compound is:

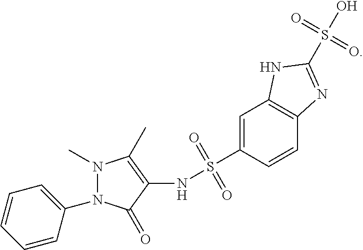 [SMILES: Cc1c(NS(=O)(=O)c2ccc3nc(S(=O)(=O)O)[nH]c3c2)c(=O)n(-c2ccccc2)n1C]

In yet another aspect, the compound is:

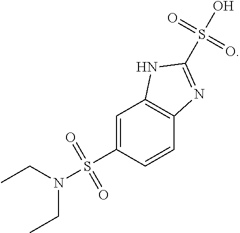 [SMILES: CCN(CC)S(=O)(=O)c1ccc2nc(S(=O)(=O)O)[nH]c2c1]

In yet another aspect, the compound is:

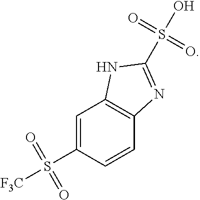 [SMILES: O=S(=O)(O)c1nc2ccc(S(=O)(=O)C(F)(F)F)cc2[nH]1]

In yet another aspect, the compound is:

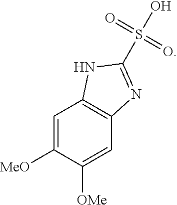 [SMILES: COc1cc2nc(S(=O)(=O)O)[nH]c2cc1OC]

In yet another aspect, the compound is:

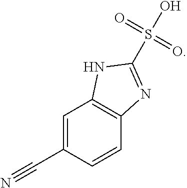 [SMILES: N#Cc1ccc2nc(S(=O)(=O)O)[nH]c2c1]

In yet another aspect, the compound is:

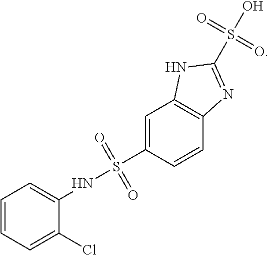 [SMILES: O=S(=O)(O)c1nc2ccc(S(=O)(=O)Nc3ccccc3Cl)cc2[nH]1]

In yet another aspect, the compound is:

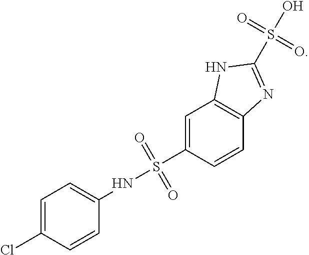 [SMILES: O=S(=O)(O)c1nc2ccc(S(=O)(=O)Nc3ccc(Cl)cc3)cc2[nH]1]

In yet another aspect, the compound is:

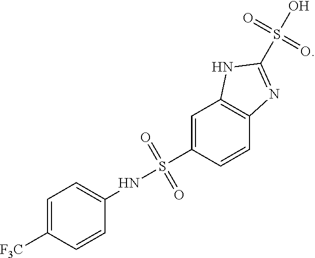 [SMILES: O=S(=O)(O)c1nc2ccc(S(=O)(=O)Nc3ccc(C(F)(F)F)cc3)cc2[nH]1]

In yet another aspect, the compound is:

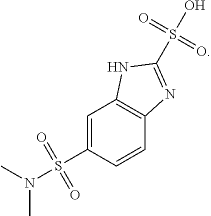 [SMILES: CN(C)S(=O)(=O)c1ccc2nc(S(=O)(=O)O)[nH]c2c1]

In yet another aspect, the compound is:

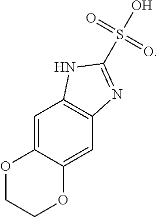 [SMILES: O=S(=O)(O)c1nc2cc3c(cc2[nH]1)OCCO3]

In yet another aspect, the compound is:

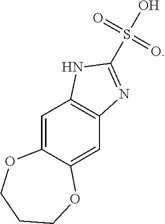 [SMILES: O=S(=O)(O)c1nc2cc3c(cc2[nH]1)OCCCO3]

In one aspect, the compound is selected from:

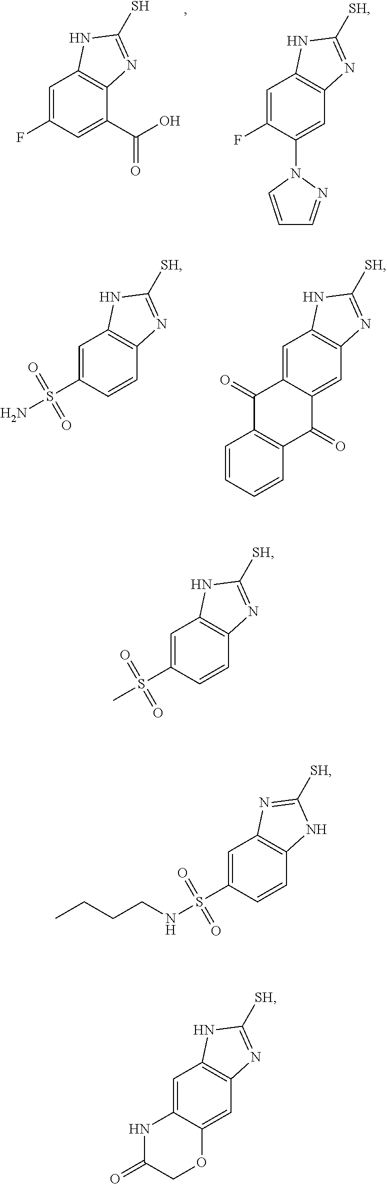

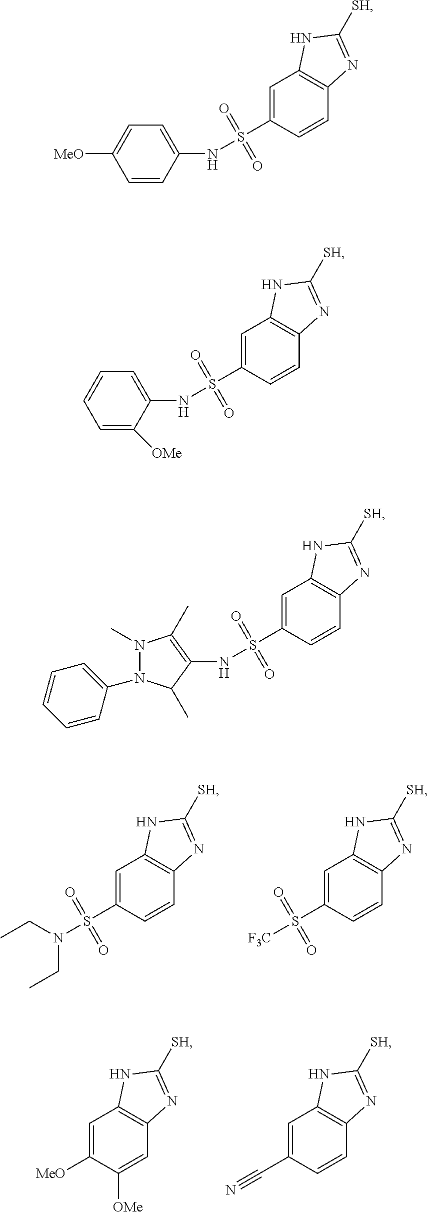

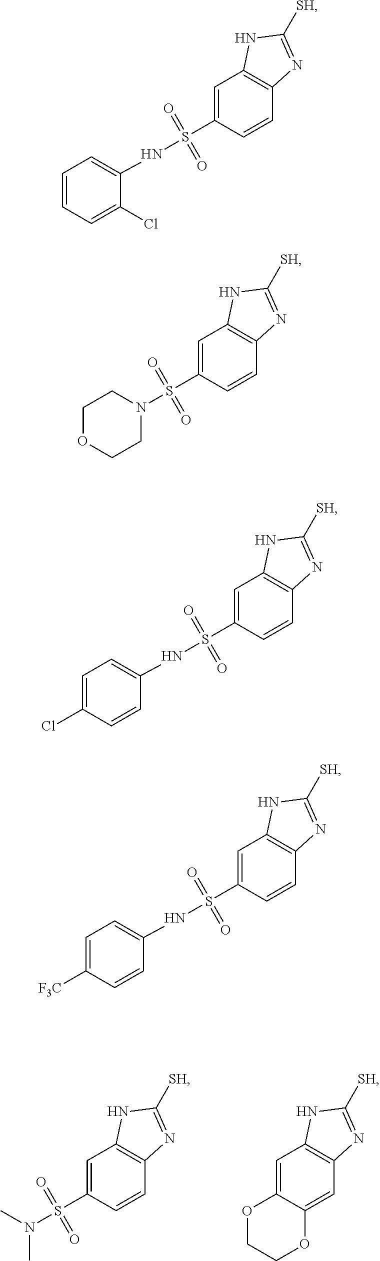 [SMILES: O=S(=O)(Nc1ccccc1Cl)c1ccc2nc(S)[nH]c2c1 ; O=S(=O)(c1ccc2nc(S)[nH]c2c1)N1CCOCC1 ; O=S(=O)(Nc1ccc(Cl)cc1)c1ccc2nc(S)[nH]c2c1 ; O=S(=O)(Nc1ccc(C(F)(F)F)cc1)c1ccc2nc(S)[nH]c2c1 ; CN(C)S(=O)(=O)c1ccc2nc(S)[nH]c2c1 ; Sc1nc2cc3c(cc2[nH]1)OCCO3]

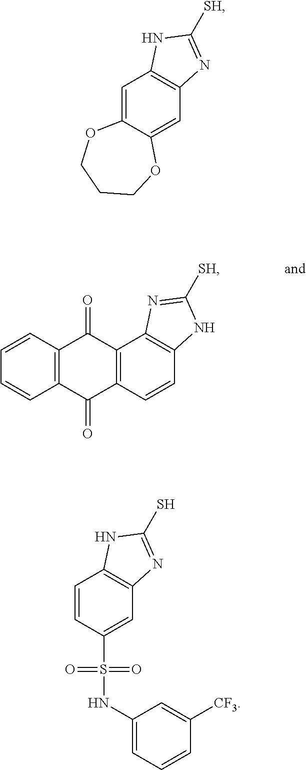 [SMILES: Sc1nc2cc3c(cc2[nH]1)OCCCO3 ; O=C1c2ccccc2C(=O)c2c1ccc1[nH]c(S)nc21 ; "and" ; O=S(=O)(Nc1cccc(C(F)(F)F)c1)c1ccc2[nH]c(S)nc2c1]

In another aspect, the compound is:

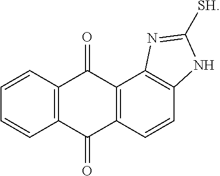 [SMILES: O=C1c2ccccc2C(=O)c2c1ccc1[nH]c(S)nc21]

In another aspect, the compound is:

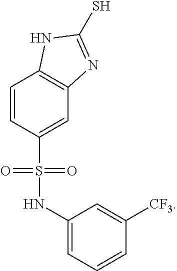 [SMILES: O=S(=O)(Nc1cccc(C(F)(F)F)c1)c1ccc2[nH]c(S)nc2c1]

In another aspect, the compound is:

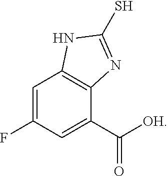 [SMILES: O=C(O)c1cc(F)cc2[nH]c(S)nc12]

In yet another aspect, the compound is:

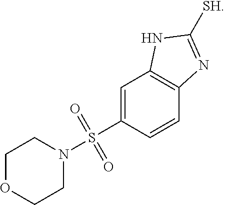 [SMILES: O=S(=O)(c1ccc2nc(S)[nH]c2c1)N1CCOCC1]

In yet another aspect, the compound is:

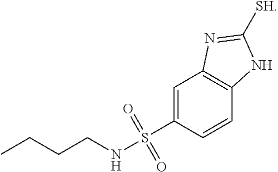 [SMILES: CCCCNS(=O)(=O)c1ccc2[nH]c(S)nc2c1]

In yet another aspect, the compound is:

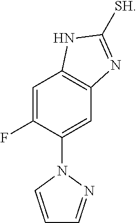 [SMILES: Fc1cc2[nH]c(S)nc2cc1-n1cccn1]

In yet another aspect, the compound is:

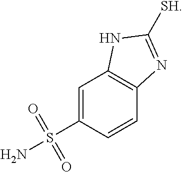 [SMILES: NS(=O)(=O)c1ccc2nc(S)[nH]c2c1]

In yet another aspect, the compound is:

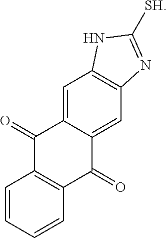 [SMILES: O=C1c2ccccc2C(=O)c2cc3[nH]c(S)nc3cc21]

In yet another aspect, the compound is:

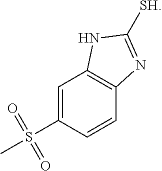

In yet another aspect, the compound is:

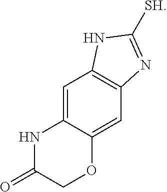 [SMILES: O=C1COc2cc3nc(S)[nH]c3cc2N1]

In yet another aspect, the compound is:

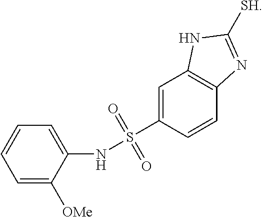 [SMILES: COc1ccccc1NS(=O)(=O)c1ccc2nc(S)[nH]c2c1]

In yet another aspect, the compound is:

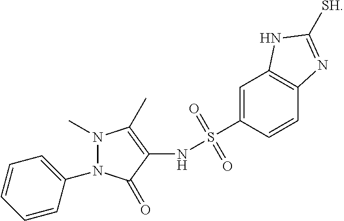 [SMILES: Cc1c(NS(=O)(=O)c2ccc3nc(S)[nH]c3c2)c(=O)n(-c2ccccc2)n1C]

In yet another aspect, the compound is:

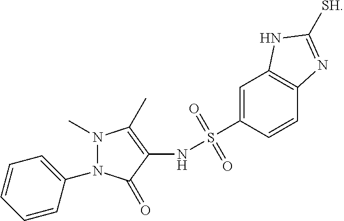 [SMILES: Cc1c(NS(=O)(=O)c2ccc3nc(S)[nH]c3c2)c(=O)n(-c2ccccc2)n1C]

In yet another aspect, the compound is:

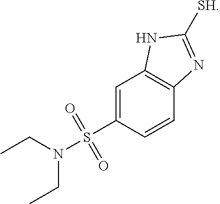 [SMILES: CCN(CC)S(=O)(=O)c1ccc2nc(S)[nH]c2c1]

In yet another aspect, the compound is:

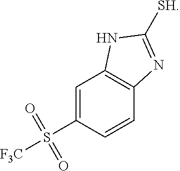 [SMILES: O=S(=O)(c1ccc2nc(S)[nH]c2c1)C(F)(F)F]

In yet another aspect, the compound is:

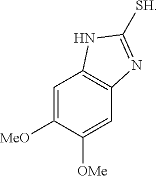 [SMILES: COc1cc2nc(S)[nH]c2cc1OC]

In yet another aspect, the compound is:

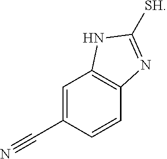 [SMILES: N#Cc1ccc2nc(S)[nH]c2c1]

In yet another aspect, the compound is:

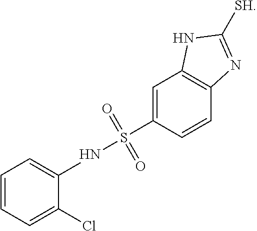 [SMILES: O=S(=O)(Nc1ccccc1Cl)c1ccc2nc(S)[nH]c2c1]

In yet another aspect, the compound is:

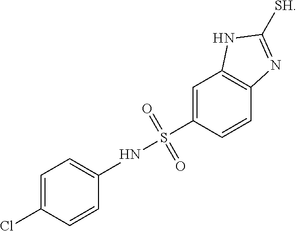 [SMILES: O=S(=O)(Nc1ccc(Cl)cc1)c1ccc2nc(S)[nH]c2c1]

In yet another aspect, the compound is:

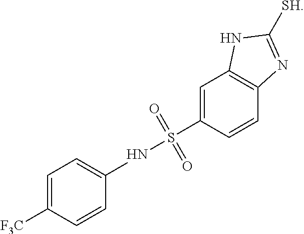 [SMILES: O=S(=O)(Nc1ccc(C(F)(F)F)cc1)c1ccc2nc(S)[nH]c2c1]

In yet another aspect, the compound is:

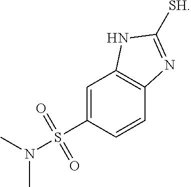 [SMILES: CN(C)S(=O)(=O)c1ccc2nc(S)[nH]c2c1]

In yet another aspect, the compound is:

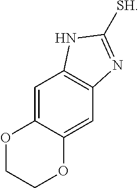 [SMILES: Sc1nc2cc3c(cc2[nH]1)OCCO3]

In yet another aspect, the compound is:

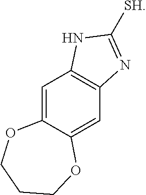 [SMILES: Sc1nc2cc3c(cc2[nH]1)OCCCO3]

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

19. Use of Compounds

In one aspect, the disclosed compounds can be administered to an animal. In a still further aspect, the animal is a mammal. In a yet further aspect, the mammal is a human. In a further aspect, the mammal is a mouse. In a yet further aspect, the mammal is a rodent. In a yet further aspect, the animal is a fish or a bird.

In a further aspect, the disclosed compounds treat or prevent a microbial infection when administered at a dose of greater than about 5 mg per day in a human. In a further aspect, the disclosed compounds treat or prevent a microbial infection when administered at a dose of greater than about 10 mg per day in a human. In a further aspect, the disclosed compounds treat or prevent a microbial infection when administered at a dose of greater than about 25 mg per day in a human. In a further aspect, the disclosed compounds treat or prevent a microbial infection when administered at a dose of greater than about 50 mg per day in a human. In a further aspect, the disclosed compounds treat or prevent a microbial infection when administered at a dose of greater than about 75 mg per day in a human. In a further aspect, the disclosed compounds treat or prevent a microbial infection when administered at a dose of greater than about 100 mg per day in a human. In a further aspect, the disclosed compounds treat or prevent a microbial infection when administered at a dose of greater than about 150 mg per day in a human. In a further aspect, the disclosed treat or prevent a microbial infection when administered at a dose of greater than about 200 mg per day in a human. In a further aspect, the disclosed compounds treat or prevent a microbial infection when administered at a dose of greater than about 250 mg per day in a human. In a yet further aspect, the disclosed compounds treat or prevent a microbial infection when administered at a dose of greater than about 300 mg per day in a human. In a still further aspect, the disclosed compounds treat or prevent a microbial infection when administered at a dose of greater than about 400 mg per day in a human. In an even further aspect, the disclosed compounds treat or prevent a microbial infection when administered at a dose of greater than about 500 mg per day in a human. In a further aspect, the disclosed compounds treat or prevent a microbial infection when administered at a dose of greater than about 750 mg per day in a human. In a yet further aspect, the disclosed compounds treat or prevent a microbial infection when administered at a dose of greater than about 1000 mg per day in a human. In a still further aspect, the disclosed compounds treat or prevent a microbial infection when administered at an a of greater than about 1500 mg per day in a human. In an even further aspect, the disclosed compounds treat or prevent a microbial infection when administered at a dose of greater than about 2000 mg per day in a human.

In a further aspect, the disclosed compounds treat or prevent a microbial infection when administered at an oral dose of greater than about 5 mg per day in a human. In a further aspect, the disclosed compounds treat or prevent a microbial infection when administered at an oral dose of greater than about 10 mg per day in a human. In a further aspect, the disclosed compounds treat or prevent a microbial infection when administered at an oral dose of greater than about 25 mg per day in a human. In a further aspect, the disclosed compounds treat or prevent a microbial infection when administered at an oral dose of greater than about 50 mg per day in a human. In a further aspect, the disclosed compounds treat or prevent a microbial infection when administered at an oral dose of greater than about 75 mg per day in a human. In a further aspect, the disclosed compounds treat or prevent a microbial infection when administered at an oral dose of greater than about 100 mg per day in a human. In a further aspect, the disclosed compounds treat or prevent a microbial infection when administered at an oral dose of greater than about 150 mg per day in a human. In a further aspect, the disclosed treat or prevent a microbial infection when administered at an oral dose of greater than about 200 mg per day in a human. In a further aspect, the disclosed compounds treat or prevent a microbial infection when administered at an oral dose of greater than about 250 mg per day in a human. In a yet further aspect, the disclosed compounds treat or prevent a microbial infection when administered at an oral dose of greater than about 300 mg per day in a human. In a still further aspect, the disclosed compounds treat or prevent a microbial infection when administered at an oral dose of greater than about 400 mg per day in a human. In an even further aspect, the disclosed compounds treat or prevent a microbial infection when administered at an oral dose of greater than about 500 mg per day in a human. In a further aspect, the disclosed compounds treat or prevent a microbial infection when administered at an oral dose of greater than about 750 mg per day in a human. In a yet further aspect, the disclosed compounds treat or prevent a microbial infection when administered at an oral dose of greater than about 1000 mg per day in a human. In a still further aspect, the disclosed compounds treat or prevent a microbial infection when administered at an oral dose of greater than about 1500 mg per day in a human. In an even further aspect, the disclosed compounds treat or prevent a microbial infection when administered at an oral dose of greater than about 2000 mg per day in a human.

It is contemplated that one or more compounds can optionally be omitted from the disclosed invention.

### C. PHARMACEUTICAL AND NEUTRACEUTICAL COMPOSITIONS

In one aspect, the invention relates to pharmaceutical and neutraceutical compositions comprising the disclosed compounds. That is, a pharmaceutical composition can be provided comprising a therapeutically effective amount of at least one disclosed compound. In another example, a pharmaceutical composition can be provided comprising a prophylactically effective amount of at least one disclosed compound. In yet another example, a neutraceutical composition can be provided comprising a neutraceutically effective amount of at least one disclosed compound

In one aspect, the invention relates to pharmaceutical compositions comprising a pharmaceutically acceptable carrier and a compound, wherein the compound is present in an effective amount. In another aspect, the invention relates to neutraceutical compositions comprising a neutraceutically acceptable carrier and a compound, wherein the compound is present in an effective amount. In one example, the compound can be a tomatidine analog.

In one aspect, the compound is present in an amount greater than about an amount selected from 5 mg, 10 mg, 25 mg, 50 mg, 100 mg, 150 mg, 200 mg, 250 mg, 300 mg, 400, mg, 500 mg, 750 mg, 1000 mg, 1,500 mg, or 2,000 mg.

The disclosed pharmaceutical compositions can further comprise one or more antibiotic. In one aspect, the pharmaceutical compositions can further comprise a penicillin, a cephalosporin, a vanomycin, a bacitracin, a monobactams, a fosfomycin, a cycloserine, or a polymyxin, or a mixture thereof. In another aspect, the pharmaceutical compositions can further comprise a penicillin. In yet another aspect, the pharmaceutical compositions can further comprise a cephalosporin. In yet another aspect, the pharmaceutical compositions can further comprise a vanomycin. In yet another aspect, the pharmaceutical compositions can further comprise a bacitracin. In yet another aspect, the pharmaceutical compositions can further comprise a monobactams. In yet another aspect, the pharmaceutical compositions can further comprise a fosfomycin. In yet another aspect, the pharmaceutical compositions can further comprise a cycloserine. In yet another aspect, the pharmaceutical compositions can further comprise a polymyxin. In a further aspect, the amount of the one or more antibiotics is a therapeutically effective amount. In a still further aspect, the amount is a prophylactically effective amount.

In a further aspect, pharmaceutical or neutraceutical composition is administered to an animal. In a still further aspect, the animal is a mammal, fish or bird. In a yet further aspect, the mammal is a primate. In a still further aspect, the mammal is a human. In an even further aspect, the human is a patient.

In a further aspect, the pharmaceutical composition is administered following identification of the mammal in need of treatment of a microbial infection. In a still further aspect, the pharmaceutical composition is administered following identification of the mammal in need of prevention of a microbial infection. In an even further aspect, the mammal has been diagnosed with a need for treatment of a microbial infection to the administering step.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” and “neutraceutically acceptable salts” refers to salts prepared from pharmaceutically or neutraceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines Other pharmaceutically or neutraceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared thereof, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, or neutraceutically acceptable salts thereof, of this invention can be combined as the active ingredient in intimate admixture with a pharmaceutical carrier or neutraceutical carrier according to conventional pharmaceutical compounding techniques or conventional neutraceutical compounding techniques. The carrier can take a wide variety of forms depending on the form of preparation desired for administration, e.g., oral or parenteral (including intravenous). Thus, the pharmaceutical compositions or neutraceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

Thus, the neutraceutical compositions of this invention can include a neutraceutically acceptable carrier and a compound or a neutraceutically acceptable salt of the compounds of the invention. The compounds of the invention, or neutraceutically acceptable salts thereof, can also be included in neutraceutical compositions in combination with one or more other therapeutically or neutraceutically active compounds.

The pharmaceutical carrier or neutraceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions or neutraceutical compositions of the present invention comprise a compound of the invention (or pharmaceutically or neutraceutically acceptable salts thereof) as an active ingredient, a pharmaceutically acceptable carrier or neutraceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carriers) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment conditions which require treatment of a microbial infection the level will generally be about 0.01 to 500 mg per kg patient body weight per day and can be administered in single or multiple doses. Preferably, the dosage level will be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0, or 5.0 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease or infection undergoing therapy.

The present invention is further directed to a method for the manufacture of a medicament for modulating cellular activity related to muscle health, muscle function, and/or healthy aging muscles (e.g., treatment of one or more disorders associated with muscle dysfunction or atrophy) in mammals (e.g., humans) comprising combining one or more disclosed compounds, products, or compositions with a pharmaceutically acceptable carrier or diluent. Thus, in one aspect, the invention relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

### D. METHODS OF MAKING THE COMPOUNDS

In one aspect, the invention relates to methods of making compounds useful to treat or prevent microbial infections, such as bacterial infections.

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Route I and Route II, as described and exemplified below. The following examples are provided so that the invention might be more fully understood, are illustrative only, and should not be construed as limiting.

In one aspect, the disclosed compounds comprise the products of the synthetic methods described herein. In a further aspect, the disclosed compounds comprise a compound produced by a synthetic method described herein. In a still further aspect, the invention comprises a pharmaceutical composition comprising a therapeutically effective amount of the product of the disclosed methods and a pharmaceutically acceptable carrier. In a still further aspect, the invention comprises a method for manufacturing a medicament comprising combining at least one compound of any of disclosed compounds or at least one product of the disclosed methods with a pharmaceutically acceptable carrier or diluent.

1. Intermediate Route I

In one aspect, 1H-benzo[d]imidazole-2-thiols can be prepared as shown below.

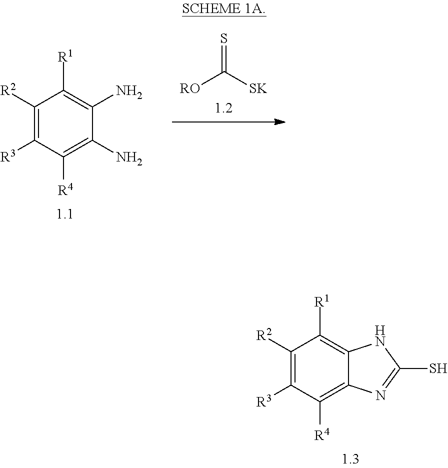

Compounds are represented in generic form, with substituents as noted in compound descriptions elsewhere herein. A more specific example is set forth below.

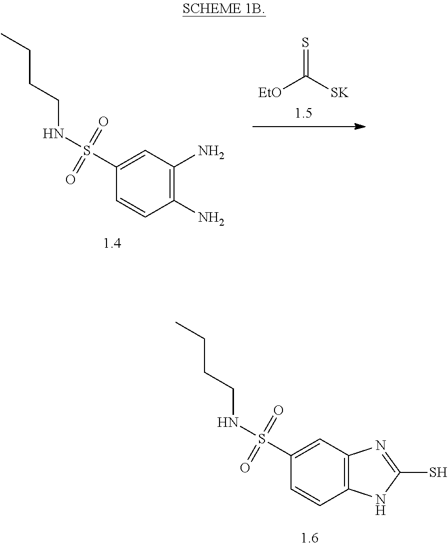

In one aspect, compounds of type 1.2, and similar compounds, can be prepared according to reaction Scheme 1B above. Thus, compounds of type 1.6 can be prepared by a coupling reaction of an appropriate diamine, e.g., 1.4 as shown above. Appropriate diamines are commercially available or prepared by methods known to one skilled in the art. The coupling reaction is carried out in the presence of an appropriate carbonodithioate, e.g., 1.5 as shown above, which is commercially available or prepared by methods known to one skilled in the art. As can be appreciated by one skilled in the art, the above reaction provides an example of a generalized approach wherein compounds similar in structure to the specific reactants above (compounds similar to compounds of type 1.4 and 1.5), can be substituted in the reaction to provide 1H-benzo[d]imidazole-2-thiols similar to Formula 1.3.

2. Intermediate Route II

In one aspect, 1H-benzo[d]imidazole-2-thiols can be prepared as shown below.

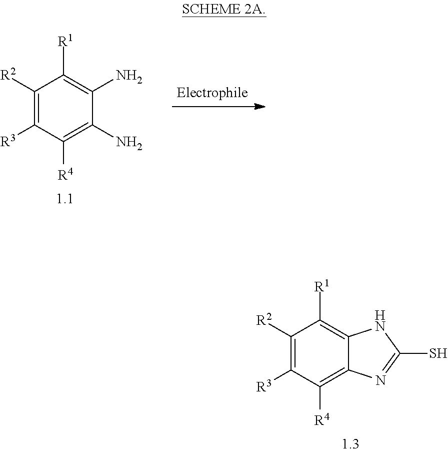

Compounds are represented in generic form, with substituents as noted in compound descriptions elsewhere herein. A more specific example is set forth below.

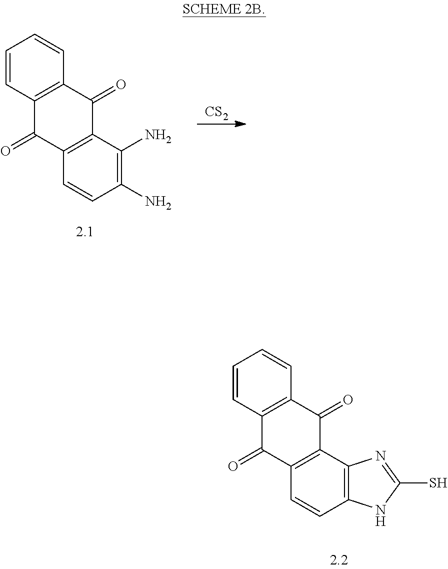

In one aspect, compounds of type 1.3, and similar compounds, can be prepared according to reaction Scheme 2B above. Thus, compounds of type 2.2 can be prepared by a cyclization reaction of an appropriate diamine, e.g., 2.1 as shown above. Appropriate diamines are commercially available or prepared by methods known to one skilled in the art. The cyclization reaction is carried out in the presence of an appropriate electrophile, e.g., cesium carbonate as shown above. As can be appreciated by one skilled in the art, the above reaction provides an example of a generalized approach wherein compounds similar in structure to the specific reactants above (compounds similar to compounds of type 2.1), can be substituted in the reaction to provide 1H-benzo[d]imidazole-2-thiols similar to Formula 1.3.

3. Analog Route I

In one aspect, 1H-benzo[d]imidazole-2-sulfonic acids can be prepared as shown below.

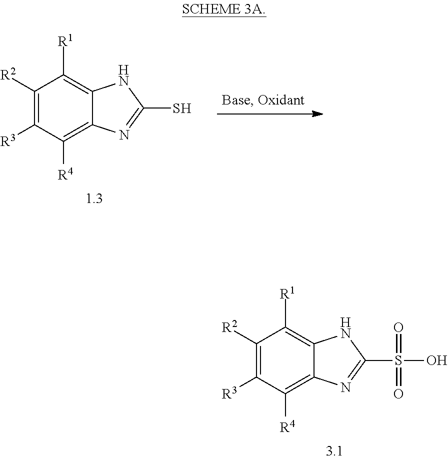

Compounds are represented in generic form, with substituents as noted in compound descriptions elsewhere herein. A more specific example is set forth below.

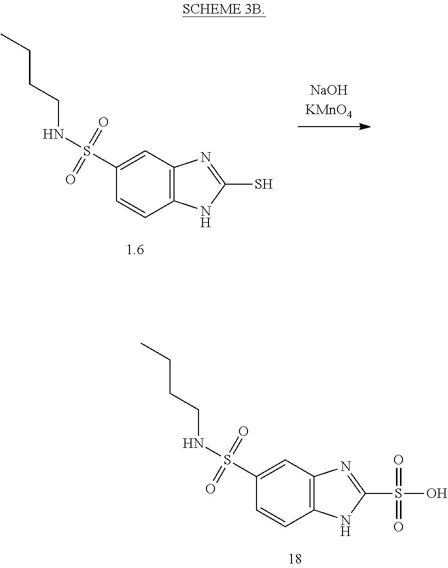 [SMILES: CCCCNS(=O)(=O)c1ccc2[nH]c(S)nc2c1 ; CCCCNS(=O)(=O)c1ccc2[nH]c(S(=O)(=O)O)nc2c1]

In one aspect, compounds of type 3.1, and similar compounds, can be prepared according to reaction Scheme 3B above. Thus, compounds of type 18 can be prepared by oxidation of an appropriate thiol, e.g., 1.6 as shown above. Appropriate thiols are commercially available or prepared by methods known to one skilled in the art. The oxidation is carried out in the presence of an appropriate base, e.g., sodium hydroxide, with an appropriate oxidant, e.g., potassium permanganate. As can be appreciated by one skilled in the art, the above reaction provides an example of a generalized approach wherein compounds similar in structure to the specific reactants above (compounds similar to compounds of type 1.6), can be substituted in the reaction to provide 1H-benzo[d]imidazole-2-sulfonic acids similar to Formula 18.

4. Analog Route II

In one aspect, 1H-benzo[d]imidazole-2-sulfonic acids can be prepared as shown below.

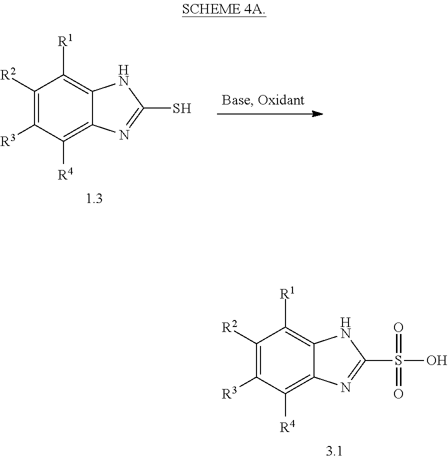

Compounds are represented in generic form, with substituents as noted in compound descriptions elsewhere herein. A more specific example is set forth below.

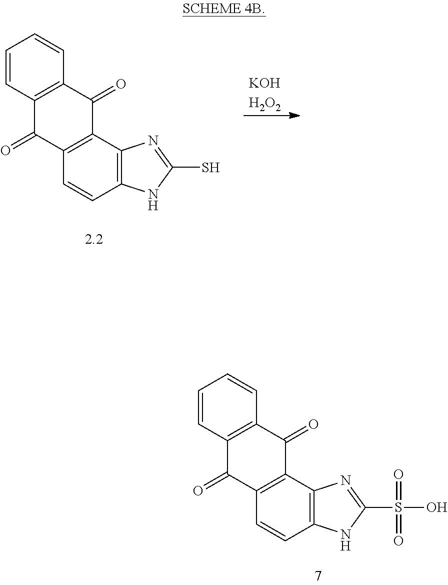 [SMILES: O=C1c2ccccc2C(=O)c2c1ccc1[nH]c(S)nc21 ; "KOHH2O2" ; O=C1c2ccccc2C(=O)c2c1ccc1[nH]c(S(=O)(=O)O)nc21]

In one aspect, compounds of type 3.1, and similar compounds, can be prepared according to reaction Scheme 4B above. Thus, compounds of type 7 can be prepared by oxidation of an appropriate thiol, e.g., 2.2 as shown above. Appropriate thiols are commercially available or prepared by methods known to one skilled in the art. The oxidation is carried out in the presence of an appropriate base, e.g., potassium hydroxide, with an appropriate oxidant, e.g., hydrogen peroxide. As can be appreciated by one skilled in the art, the above reaction provides an example of a generalized approach wherein compounds similar in structure to the specific reactants above (compounds similar to compounds of type 2.2), can be substituted in the reaction to provide 1H-benzo[d]imidazole-2-sulfonic acids similar to Formula 7.

### E. METHODS OF USING THE COMPOUNDS AND COMPOSITIONS

1. Antimicrobial

An antimicrobial compound or composition is a substance that kills or inhibits the growth of microbes, such as bacteria.

The disclosed compounds and compositions are antimicrobial compounds and compositions and can be used as single agents or in combination with one or more other antibiotics to treat or prevent a microbial infection, such a bacterial infection. Accordingly, where the combination of antibiotics together are safer or more effective than either antibiotic alone. The other antibiotics(s) can be administered by a route and in an amount commonly used therefore, contemporaneously or sequentially with a disclosed compound. When a disclosed compound is used contemporaneously with one or more other antibiotics, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

Systemic administration of one or more disclosed compounds (e.g., by parenteral injection or by oral consumption) can be used to treat or prevent a microbial infection. Local administration of a disclosed compound (by a topical route or localized injection) can be used to treat or prevent a microbial infection, as can be required following a localized injury or surgery. A disclosed compound or salt thereof can be administered at about 10 mg/day to 10 g/day.

2. Co-Administration

In one aspect, the disclosed compounds and pharmaceutical compositions can be co-administered with one or more antibiotics. In one aspect, the disclosed compounds and pharmaceutical compositions can be co-administered with a penicillin, a cephalosporin, a vanomycin, a bacitracin, a monobactams, a fosfomycin, a cycloserine, or a polymyxin, or a mixture thereof. In another aspect, the disclosed compounds and pharmaceutical compositions can be co-administered with a penicillin. In yet another aspect, the disclosed compounds and pharmaceutical compositions can be co-administered with a cephalosporin. In yet another aspect, the disclosed compounds and pharmaceutical compositions can be co-administered with a vanomycin. In yet another aspect, the disclosed compounds and pharmaceutical compositions can be co-administered with a bacitracin. In yet another aspect, the disclosed compounds and pharmaceutical compositions can be co-administered with a monobactams. In yet another aspect, the disclosed compounds and pharmaceutical compositions can be co-administered with a fosfomycin. In yet another aspect, the disclosed compounds and pharmaceutical compositions can be co-administered with a cycloserine. In yet another aspect, the disclosed compounds and pharmaceutical compositions can be co-administered with a polymyxin. In a further aspect, the disclosed compounds and pharmaceutical compositions can be co-administered with an effective amount of the antibiotic. In a still further aspect, the amount is a prophylactically effective amount of the antibiotic.

3. Treatment Methods

The compounds disclosed herein are useful for treating or preventing a microbial infection, such as a bacterial infection. The bacterial infection can be caused by Bacillus anthracis, Staphylococcus aureus, Bacillus subtilis, Escherichia coli, or Helicobacter pylori, or a mixture thereof. In aspect, the bacterial infection can be caused by any species of bacteria, which utilizes peptidoglycan as protection from osmotic pressure. In one aspect, the method eliminates or reduces the growth of microbes, such as bacteria. In one aspect, the microbes, such as bacteria, are present internally in an animal, such as a human.

Accordingly, the disclosed compounds and compositions also treat or prevent medical conditions associated with a microbial infection. For example, an infection of Helicobacter pylori can cause cancer, such as stomach cancer. Furthermore, an infection of Helicobacter pylori can cause duodenal ulcers. Therefore, in one aspect, the disclosed compounds and compositions can treat or prevent cancer, such as stomach cancer. In another aspect, the disclosed compounds and compositions can treat or prevent duodenal ulcers.

Thus, provided is a method comprising administering a therapeutically effective amount of a composition comprising a disclosed compound to a subject. In one aspect, the method can be a method for treating a microbial infection. In another aspect, the method can be a method for preventing a microbial infection. In yet another aspect, the method can be a method for treating a bacterial infection. In another aspect, the method can be a method for preventing a bacterial infection. In yet another aspect, the method can be a method for treating a cancer, such as stomach cancer. In yet another aspect, the method can be a method for preventing a cancer, such as stomach cancer. In yet another aspect, the method can be a method for treating a duodenal ulcer. In yet another aspect, the method can be a method for preventing the formation of a duodenal ulcer.

A. Treating or Preventing a Microbial Infection

Disclosed herein is a method of treating or preventing a microbial infection, such as a bacterial infection, in an animal, such as a human, comprising administering to the animal an effective amount of a compound disclosed herein. In one aspect, the bacterial infection can be caused by Bacillus anthracis, Staphylococcus aureus, Bacillus subtilis, Escherichia coli, or Helicobacter pylori, or a mixture thereof. In another aspect, the bacterial infection can be caused by Bacillus anthracis. In yet another aspect, the bacterial infection can be caused by Staphylococcus aureus. In yet another aspect, the bacterial infection can be caused by Bacillus subtilis. In yet another aspect, the bacterial infection can be caused by Helicobacter pylori. In another aspect, the bacterial infection can be caused by Escherichia coli. In yet another aspect, the bacterial infection can be caused by any species of bacteria, which utilizes peptidoglycan as protection from osmotic pressure.

In one aspect, the method is for treating or preventing a bacterial infection caused by Escherichia coli, in an animal, such as a human, comprising administering to the animal an effective amount of a compound having the formula:

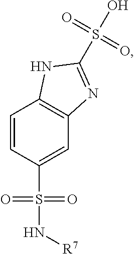

or a pharmaceutically acceptable salt, solvate, or polymorph thereof.

In one aspect, the method is for treating or preventing a bacterial infection caused by Escherichia coli, in an animal, such as a human, comprising administering to the animal an effective amount of a compound having the formula:

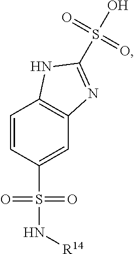

or a pharmaceutically acceptable salt, solvate, or polymorph thereof.

In one aspect, the method is for treating or preventing a bacterial infection caused by Escherichia coli, in an animal, such as a human, comprising administering to the animal an effective amount of a compound having the formula:

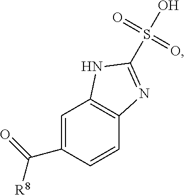

or a pharmaceutically acceptable salt, solvate, or polymorph thereof.

In one aspect, the method is for treating or preventing a bacterial infection caused by Escherichia coli, in an animal, such as a human, comprising administering to the animal an effective amount of a compound having the formula:

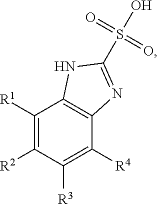

wherein R¹ and R² are covalently bonded together to form a C3-C10 cycloalkyl or C2-C7 heterocycloalkyl, or a pharmaceutically acceptable salt, solvate, or polymorph thereof

In one aspect, the method is for treating or preventing a bacterial infection caused by Escherichia coli, in an animal, such as a human, comprising administering to the animal an effective amount of a compound having the formula:

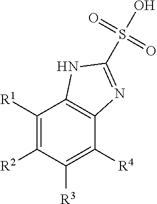

wherein R² and R³ are covalently bonded together to form a C3-C10 cycloalkyl or C2-C7 heterocycloalkyl, or a pharmaceutically acceptable salt, solvate, or polymorph thereof

In one aspect, the method is for treating or preventing a bacterial infection caused by Escherichia coli, in an animal, such as a human, comprising administering to the animal an effective amount of a compound having the formula:

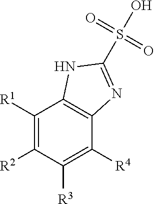

wherein R³ and R⁴ are covalently bonded together to form a C3-C10 cycloalkyl or C2-C7 heterocycloalkyl, or a pharmaceutically acceptable salt, solvate, or polymorph thereof

In one aspect, the method is for treating or preventing a bacterial infection caused by Escherichia coli, in an animal, such as a human, comprising administering to the animal an effective amount of a compound selected from the group:

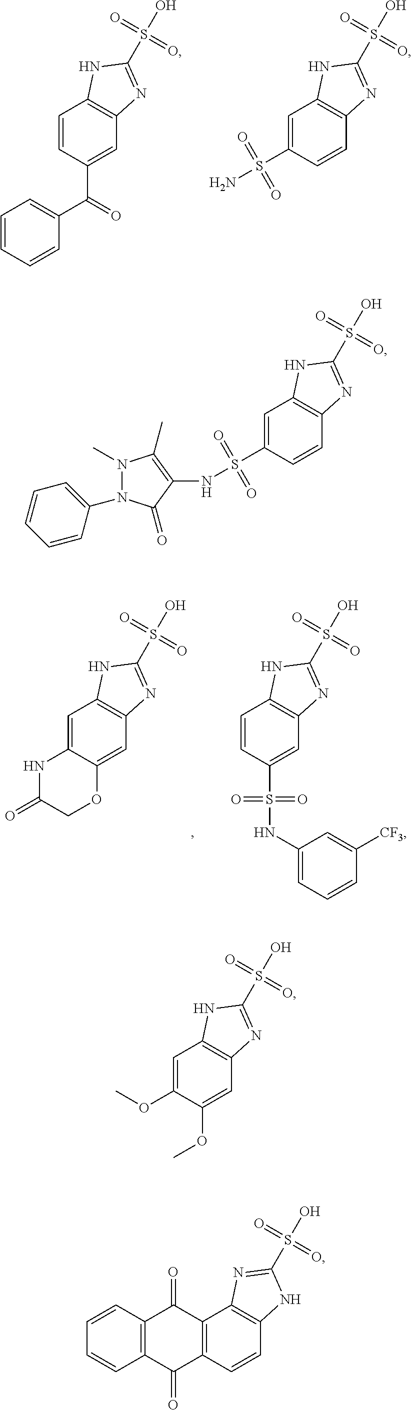

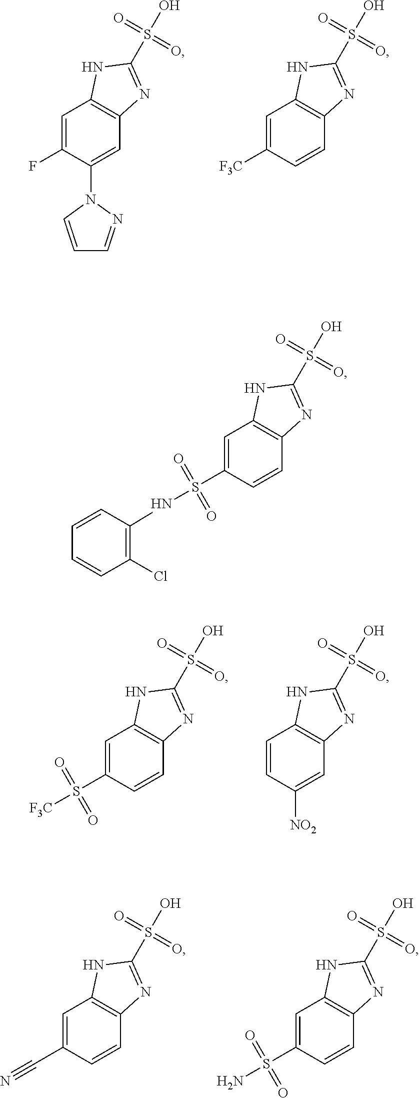

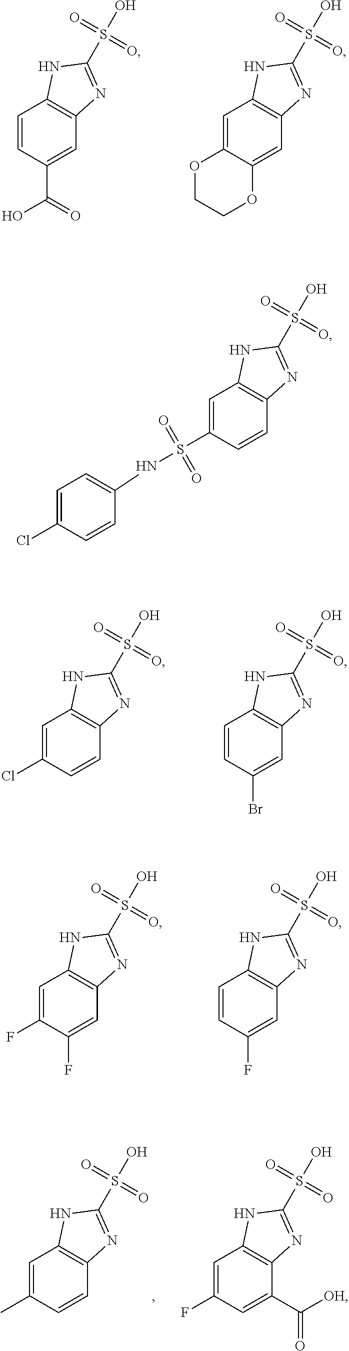

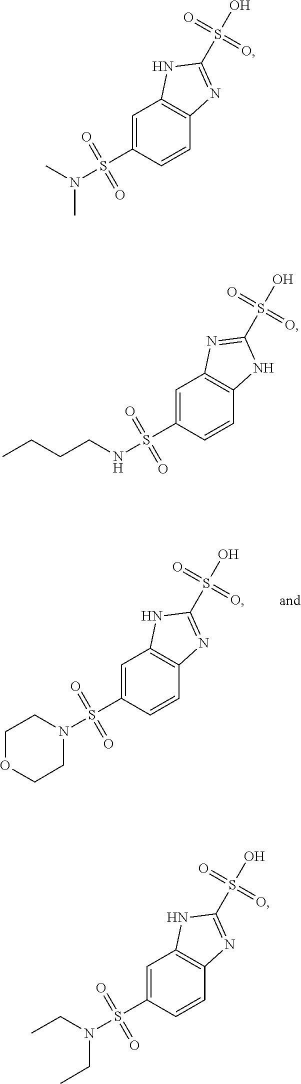 [SMILES: CN(C)S(=O)(=O)c1ccc2nc(S(=O)(=O)O)[nH]c2c1 ; CCCCNS(=O)(=O)c1ccc2[nH]c(S(=O)(=O)O)nc2c1 ; O=S(=O)(O)c1nc2ccc(S(=O)(=O)N3CCOCC3)cc2[nH]1 ; "and" ; CCN(CC)S(=O)(=O)c1ccc2nc(S(=O)(=O)O)[nH]c2c1]

or a pharmaceutically acceptable salt, solvate, or polymorph thereof.

In one aspect, the method is for treating or preventing a bacterial infection caused by Escherichia coli, in an animal, such as a human, comprising administering to the animal an effective amount of a compound selected from the group:

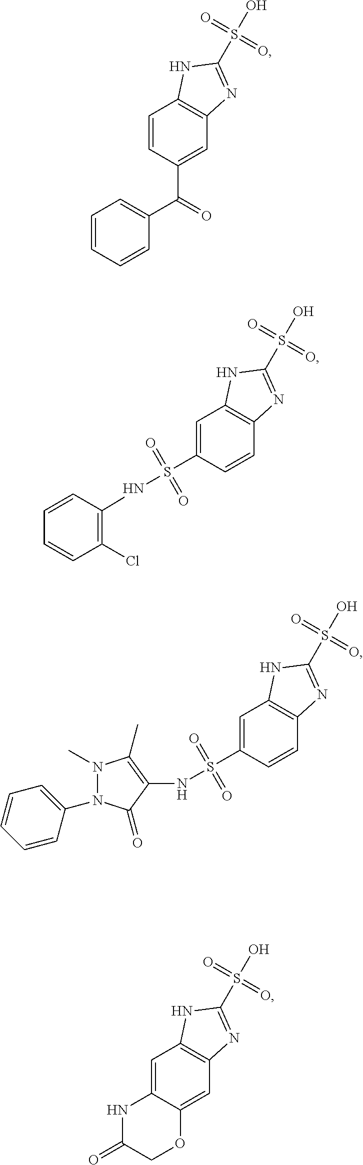 [SMILES: O=C(c1ccccc1)c1ccc2[nH]c(S(=O)(=O)O)nc2c1 ; O=S(=O)(O)c1nc2ccc(S(=O)(=O)Nc3ccccc3Cl)cc2[nH]1 ; Cc1c(NS(=O)(=O)c2ccc3nc(S(=O)(=O)O)[nH]c3c2)c(=O)n(-c2ccccc2)n1C ; O=C1COc2cc3nc(S(=O)(=O)O)[nH]c3cc2N1]

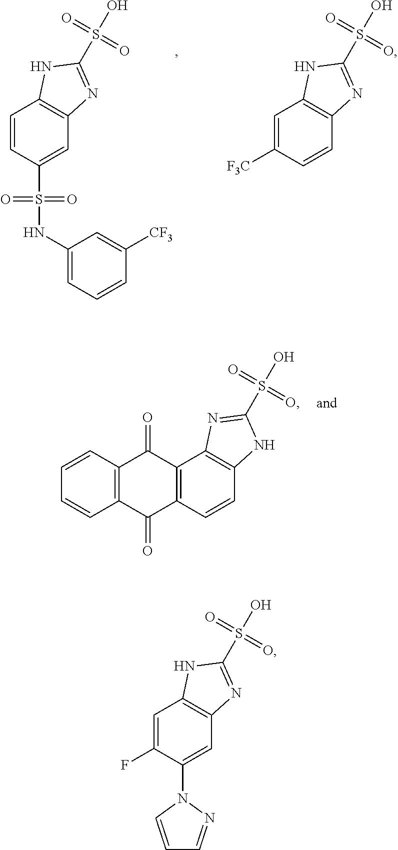 [SMILES: O=S(=O)(O)c1nc2cc(S(=O)(=O)Nc3cccc(C(F)(F)F)c3)ccc2[nH]1 ; O=S(=O)(O)c1nc2ccc(C(F)(F)F)cc2[nH]1 ; O=C1c2ccccc2C(=O)c2c1ccc1[nH]c(S(=O)(=O)O)nc21 ; "and" ; O=S(=O)(O)c1nc2cc(-n3cccn3)c(F)cc2[nH]1]

or a pharmaceutically acceptable salt, solvate, or polymorph thereof.

In one aspect, the method is for treating or preventing a bacterial infection caused by Staphylococcus aureus, in an animal, such as a human, comprising administering to the animal an effective amount of a compound having the formula:

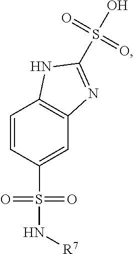

or a pharmaceutically acceptable salt, solvate, or polymorph thereof.

In one aspect, the method is for treating or preventing a bacterial infection caused by Staphylococcus aureus, in an animal, such as a human, comprising administering to the animal an effective amount of a compound having the formula:

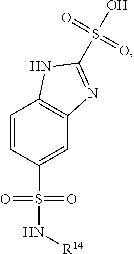

or a pharmaceutically acceptable salt, solvate, or polymorph thereof.

In one aspect, the method is for treating or preventing a bacterial infection caused by Staphylococcus aureus, in an animal, such as a human, comprising administering to the animal an effective amount of a compound having the formula:

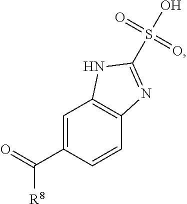

or a pharmaceutically acceptable salt, solvate, or polymorph thereof.

In one aspect, the method is for treating or preventing a bacterial infection caused by Staphylococcus aureus, in an animal, such as a human, comprising administering to the animal an effective amount of a compound having the formula:

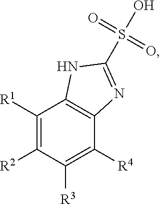

wherein R¹ and R² are covalently bonded together to form a C3-C10 cycloalkyl or C2-C7 heterocycloalkyl, or a pharmaceutically acceptable salt, solvate, or polymorph thereof.

In one aspect, the method is for treating or preventing a bacterial infection caused by Staphylococcus aureus, in an animal, such as a human, comprising administering to the animal an effective amount of a compound having the formula:

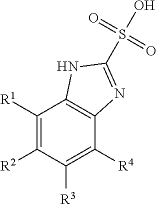

wherein R² and R³ are covalently bonded together to form a C3-C10 cycloalkyl or C2-C7 heterocycloalkyl, or a pharmaceutically acceptable salt, solvate, or polymorph thereof.

In one aspect, the method is for treating or preventing a bacterial infection caused by Staphylococcus aureus, in an animal, such as a human, comprising administering to the animal an effective amount of a compound having the formula:

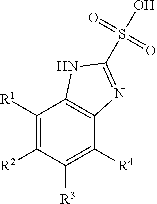

wherein R³ and R⁴ are covalently bonded together to form a C3-C10 cycloalkyl or C2-C7 heterocycloalkyl, or a pharmaceutically acceptable salt, solvate, or polymorph thereof.

In one aspect, the method is for treating or preventing a bacterial infection caused by Staphylococcus aureus, in an animal, such as a human, comprising administering to the animal an effective amount of a compound selected from the group:

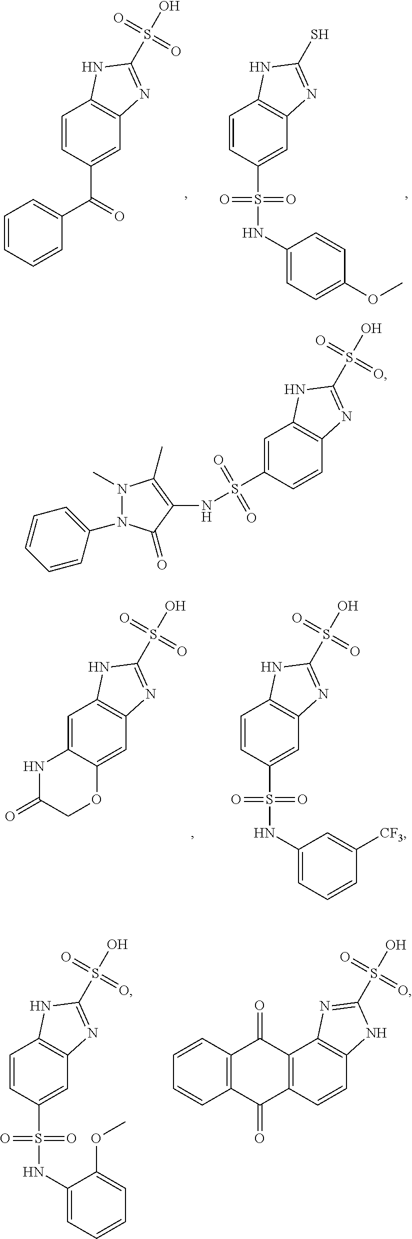

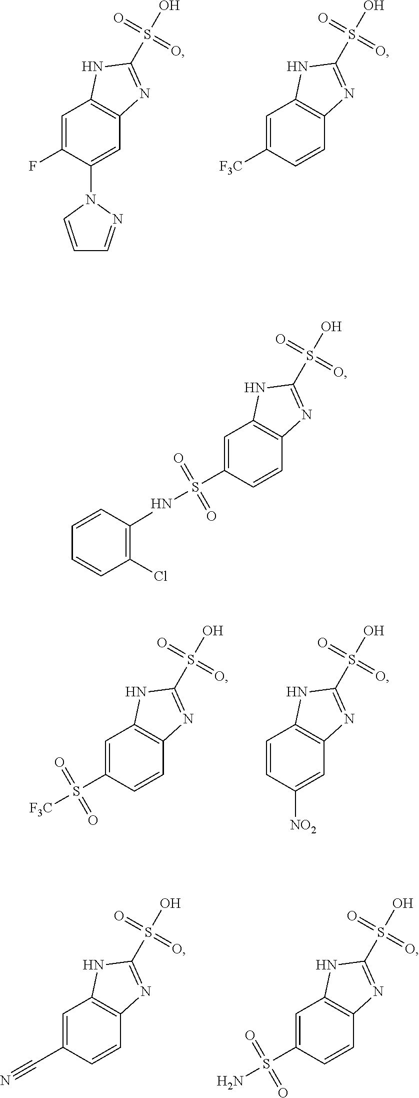

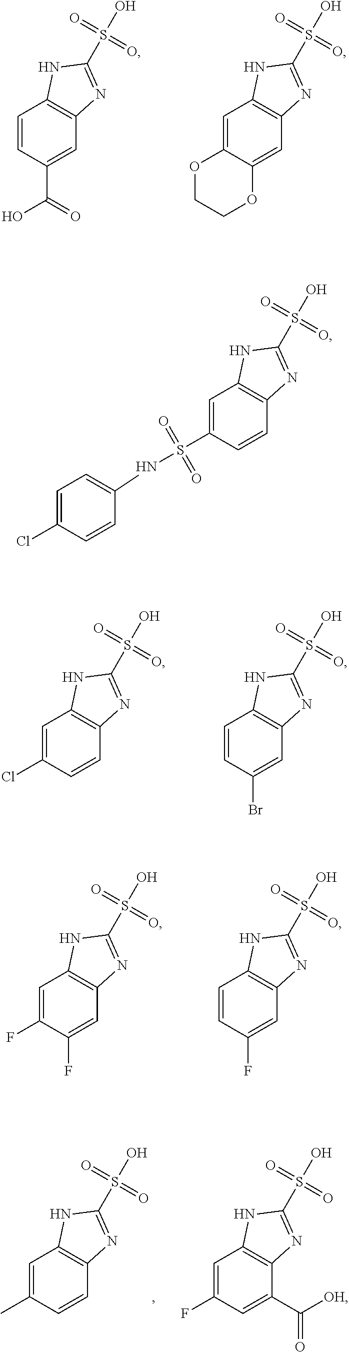

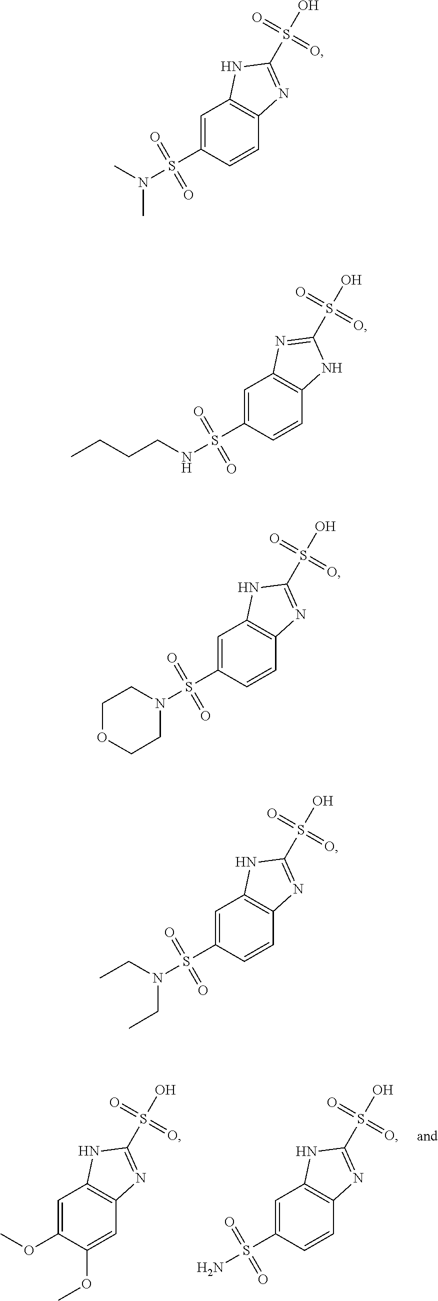

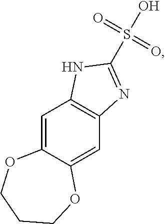 [SMILES: O=S(=O)(O)c1nc2cc3c(cc2[nH]1)OCCCO3]

or a pharmaceutically acceptable salt, solvate, or polymorph thereof.

In one aspect, the method is for treating or preventing a bacterial infection caused by Escherichia coli, in an animal, such as a human, comprising administering to the animal an effective amount of a compound selected from the group:

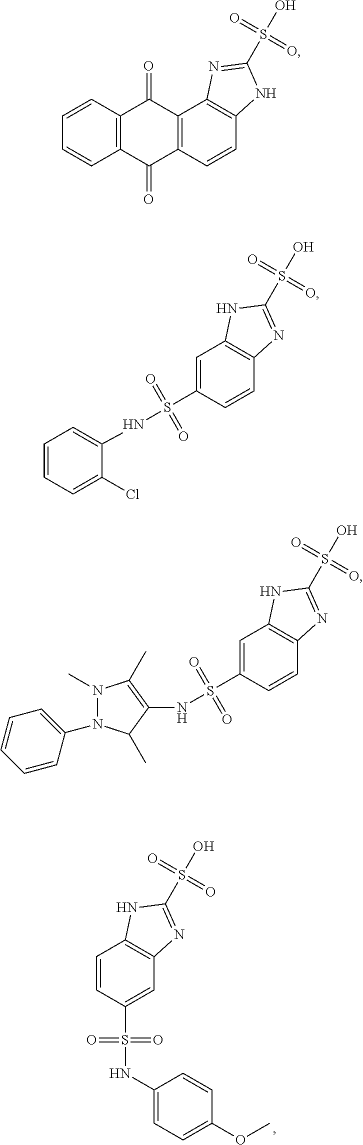 [SMILES: O=C1c2ccccc2C(=O)c2c1ccc1[nH]c(S(=O)(=O)O)nc21 ; O=S(=O)(O)c1nc2ccc(S(=O)(=O)Nc3ccccc3Cl)cc2[nH]1 ; CC1=C(NS(=O)(=O)c2ccc3nc(S(=O)(=O)O)[nH]c3c2)C(C)N(c2ccccc2)N1C ; COc1ccc(NS(=O)(=O)c2ccc3[nH]c(S(=O)(=O)O)nc3c2)cc1]

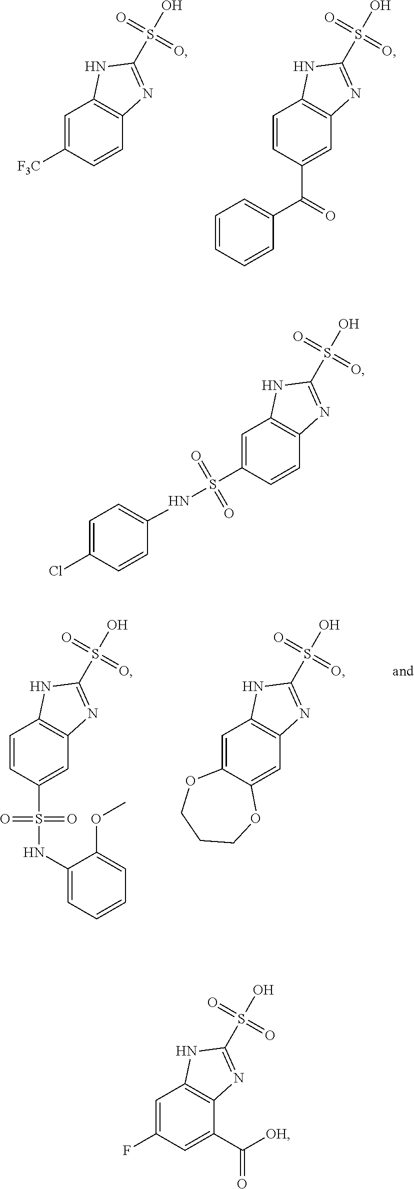

or a pharmaceutically acceptable salt, solvate, or polymorph thereof.

In one aspect, the compound is administered in an amount between about 0.01 to 500 mg per kg patient body weight per day and can be administered in single or multiple doses. Preferably, the dosage level will be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900 and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

In a further aspect, the compound administered is a disclosed compound or a product of a disclosed method of making a compound. In a yet further aspect, the invention relates to a pharmaceutical composition comprising at least one compound as disclosed herein.

In a further aspect, the compound is co-administered with an antibiotic agent as disclosed herein.

In a further aspect, the animal is a mammal, fish or bird. In a yet further aspect, the mammal is a primate. In a still further aspect, the mammal is a human. In an even further aspect, the human is a patient.

In a further aspect, the animal is a domesticated animal. In a still further aspect, the domesticated animal is a domesticated fish, domesticated crustacean, or domesticated mollusk. In a yet further aspect, the domesticated animal is poultry. In an even further aspect, the poultry is selected from chicken, turkey, duck, and goose. In a still further aspect, the domesticated animal is livestock. In a yet further aspect, the livestock animal is selected from pig, cow, horse, goat, bison, and sheep.

In a still further aspect, the method further comprises the step of identifying the animal in need of treatment or prevention of a microbial infection. In an even further aspect, the mammal has been diagnosed with a need for treatment and prevention of a microbial infection prior to the administering step.

4. Manufacture of a Medicament

In one aspect, the invention relates to a method for the manufacture of a medicament for treating or preventing a microbial infection comprising combining a therapeutically effective amount of a disclosed compound or product of a disclosed method with a pharmaceutically acceptable carrier or diluent.

In one aspect, the invention relates to a method for manufacturing a medicament associated with treating or preventing a microbial infection or the need to treat or prevent a microbial infection with a pharmaceutically acceptable carrier or diluent.

In a further aspect, the medicament comprises a disclosed compound.

5. Kits

Also disclosed herein are kit comprising one or more of the disclosed compounds, and one or more of: a) at least one antibiotic agent, b) instructions for treating a disorder associated with microbial activity, c) instructions for treating an infection, d) a penicillin, e) a cephalosporin, f) a vanomycin, g) a bacitracin, h) a monobactams, i) a fosfomycin, j) a cycloserine, or k) a polymyxin.

In one aspect, the kit further comprises at least one agent, wherein the compound and the agent are co-formulated.

In another aspect, the compound and the agent are co-packaged. The agent can be any agent as disclosed herein, known to have a side effect of a microbial infection, an agent known to increase the risk of a microbial infection, agent known to treat a microbial infection in an animal.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

6. An Article

Also provided are articles comprising one or more of the disclosed compounds. In one aspect, the article comprises a first surface. In another aspect, the first surface is an outer surface. In one aspect, the article in a medical device. The medical device can be a device present in a hospital. In yet another aspect, the article can be textile, such as clothing, for example, clothes used in a hospital. The compounds can be covalently bonded to the surface as described herein or be coated on to the surface as described herein.

### F. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Certain materials, reagents and kits were obtained from specific vendors as indicated below, and as appropriate the vendor catalog, part or other number specifying the item are indicated.

1. Scaffold Optimization

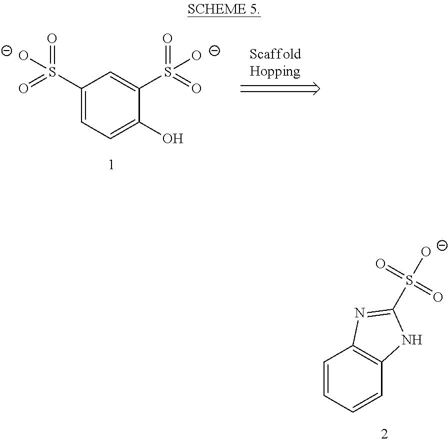 [SMILES: O=S(=O)([O-])c1ccc(O)c(S(=O)(=O)[O-])c1 ; O=S(=O)([O-])c1nc2ccccc2[nH]1]

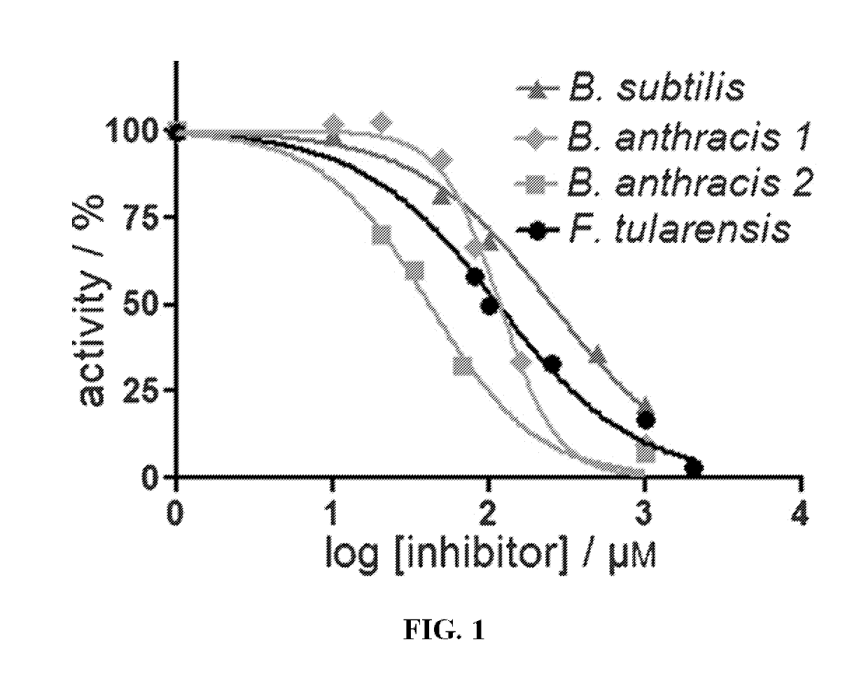

4-Hydroxy-1,3-benzenedisulfonic acid (1) (Scheme 5) was discovered in a virtual screening campaign against GR using the Chemical Computing Group lead-like library 1 million compounds) (Whalen, K. L., et al. (2010) ACS Med. Chem. Lett. 8, 9-13). The inhibitory constant against GR from B. subtilis was 58±13 p.m. Scaffold hopping to 1H-benzimidazole-2-sulfonic acid (2) increases affinity against this target to a Kᵢ value of 9±2 μm. Compound 2 also shows equal potency against two isozymes of GR from B. anthracis (RacE1 and RacE2) as well as GR from F. tularensis (Mull), two bacterial species currently considered as Tier 1 Biological Select Agents by the US Government (FIG. 1 and Table 1). The high LE of this fragment, coupled with its cross-species activity made compound 2 an ideal candidate for optimization.

TABLE 1

| Species | IC₅₀ (μM) |
|---|---|
| B. subtilis | 250 ± 20 |
| B. anthracis 1 | 120 ± 5 |
| B. anthracis 2 | 40 ± 5 |
| F. tularensis | 110 ± 12 |

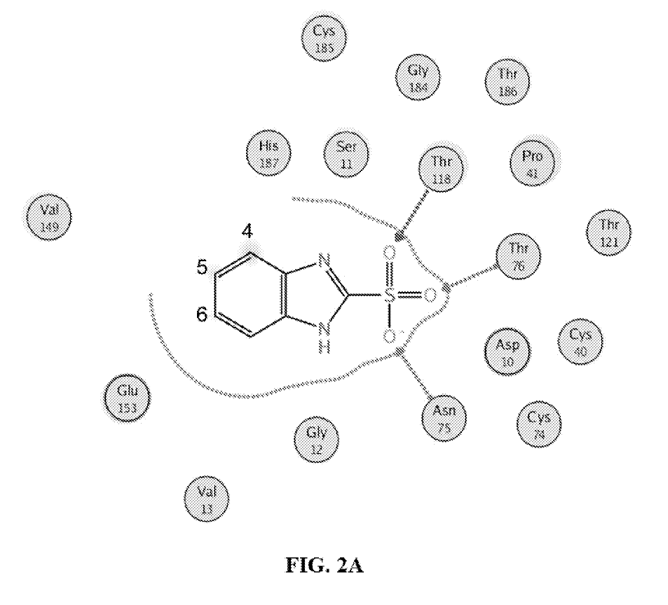

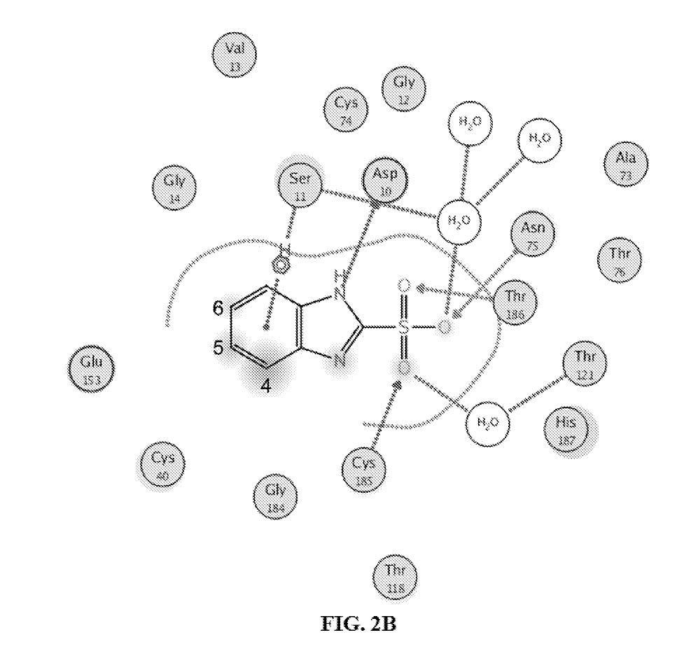

To generate a basis for rational lead optimization, a basic understanding of the physicochemical components of binding between ligand and receptor is required. As an alternative to X-ray crystallography or NMR spectroscopy, virtual docking was used to generate structural information regarding the interaction of GR and compound 2. Compound 2 was docked into GR in silico by using a previously solved crystal structure (PDB ID: 1ZUW) as the receptor. The result of docking shows compound 2 with its sulfonic acid moiety situated in the most buried region of the active site, between the catalytic cysteine residues (FIGS. 2A and 2B). The sulfonate moiety participates in several hydrogen bonding interactions with Asn75, Thr186, and Cys185. The benzene moiety also interacts with Ser11 via an OH-π interaction. These moieties both appear to contribute to the recognition of compound 2, and thus the optimization strategy focused on the addition of substituents that would produce additional interactions while preserving the original contacts. As observed by their solvent exposure and protein proximity (symbolized by light blue shading or a grey dotted line, respectively, in FIGS. 2A and 2B), carbon atoms 4, 5, and 6 within the benzene ring could serve as starting points to build on additional chemical groups without encountering steric clash from active site residues. Depending on their size, substituents added at these positions have the capacity to reach additional binding pockets proximal to the main substrate binding cleft.

2. Docking and FERM-SMD

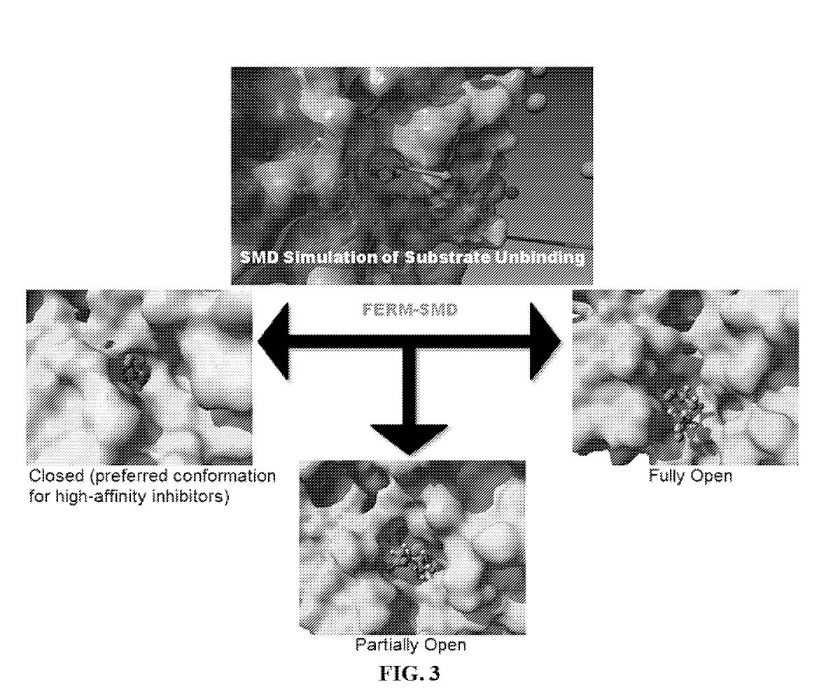

An in silico library containing compounds 3-35 (Table 1) was subjected to a hybrid ensemble docking scheme, referred to as the flexible enzyme receptor method by steered molecular dynamics (FERM-SMD), previously described by Whalen and co-workers (Whalen, K. L., et al. (2011) Mol. Inf. 30, 459-471). FIG. 3 details how unique conformations of the protein target were generated using steered MD simulations to emulate the substrate unbinding trajectory. Starting with a crystal structure of D-glutamate bound to GR, D-glutamate is pulled from the active site over the course of the simulation. In the process, the enzyme alters its structural conformation to allow substrate passage from the buried binding cleft. Three snapshots were chosen to represent three distinct structural states, distinguished by the entrance to the binding cleft: closed (corresponding to 0 ps simulation time), partially open (13.9 ps simulation time), and fully open (20 ps simulation time) (FIG. 3). Compounds were docked to all three structures using YASARA v9.11.9 (Krieger, E. (2011) YASARA Biosciences GmbH, Vienna (Austria); Krieger, E., et al. (2002) Proteins 47, 393-402), which uses an optimized version of Auto-Dock 4 (Morris, G. M., et al. (2009) J. Comput. Chem. 30, 2785-2791). Simulation cells were centered around the active site and expanded to include the residues surrounding the cleft entrance. Simulation cells had the following dimensions (in Å): “0 ps” receptor=18.75×20.19×19.29; “13.9 ps” receptor=19.08×21.56×18.07; and “20 ps” receptor=18.94×21.31×18.93. Receptorligand docking combinations that resulted in more than one high-ranking pose were visually assessed, and the pose that placed the core scaffold in a position most similar to the parent scaffold position was chosen as the “true” pose. The predicted binding affinities were adjusted according to the respective protein solvation energy (these varied greatly and affected the accuracy of binding affinity calculations) and weighted to indicate relative binding specificity to one of the three receptors. Previous studies have shown that the final score produced by FERM-SMD, deemed the FERMscore, shares a high correlation with experimental binding affinities, particularly for congeneric ligands of GR. On a set of 17 ligands, FERM-SMD has a predictive accuracy of ±1 kcal mol⁻¹ (Whalen, K. L., et al. (2011) Mol. Inf. 30, 459-471).

TABLE 2

| Compound | |
|---|---|
| No. | Structure |
| 3 | 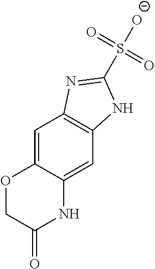 [SMILES: O=C1COc2cc3nc(S(=O)(=O)[O-])[nH]c3cc2N1] |
| 4 | 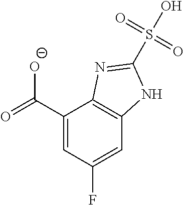 [SMILES: O=C([O-])c1cc(F)cc2[nH]c(S(=O)(=O)O)nc12] |
| 5 | 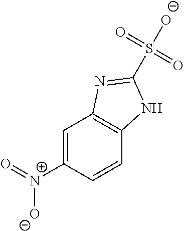 [SMILES: O=[N+]([O-])c1ccc2[nH]c(S(=O)(=O)[O-])nc2c1] |
| 6 | 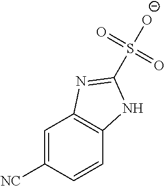 [SMILES: N#Cc1ccc2[nH]c(S(=O)(=O)[O-])nc2c1] |
| 7 | 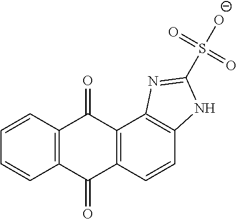 [SMILES: O=C1c2ccccc2C(=O)c2c1ccc1[nH]c(S(=O)(=O)[O-])nc21] |
| 8 | 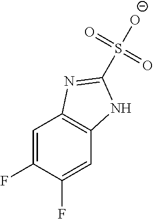 [SMILES: O=S(=O)([O-])c1nc2cc(F)c(F)cc2[nH]1] |
| 9 | 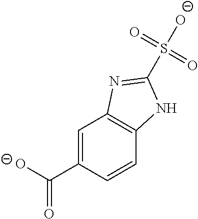 [SMILES: O=C([O-])c1ccc2[nH]c(S(=O)(=O)[O-])nc2c1] |
| 10 | 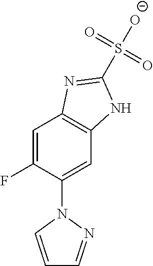 [SMILES: O=S(=O)([O-])c1nc2cc(F)c(-n3cccn3)cc2[nH]1] |
| 11 | 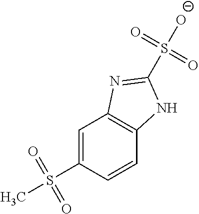 [SMILES: CS(=O)(=O)c1ccc2[nH]c(S(=O)(=O)[O-])nc2c1] |
| 12 | 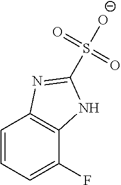 [SMILES: O=S(=O)([O-])c1nc2cccc(F)c2[nH]1] |
| 13 | 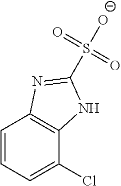 [SMILES: O=S(=O)([O-])c1nc2cccc(Cl)c2[nH]1] |
| 14 | 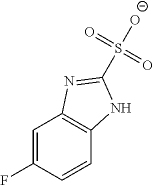 [SMILES: O=S(=O)([O-])c1nc2cc(F)ccc2[nH]1] |
| 15 | 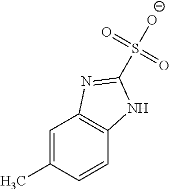 |
| 16 | 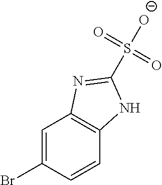 [SMILES: O=S(=O)([O-])c1nc2cc(Br)ccc2[nH]1] |
| 17 | 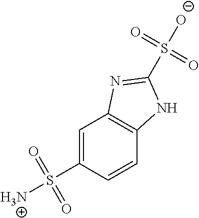 [SMILES: [NH3+]S(=O)(=O)c1ccc2[nH]c(S(=O)(=O)[O-])nc2c1] |
| 18 | 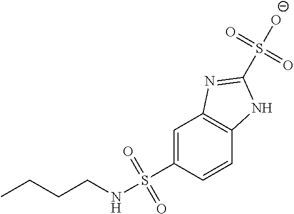 [SMILES: CCCCNS(=O)(=O)c1ccc2[nH]c(S(=O)(=O)[O-])nc2c1] |
| 19 | 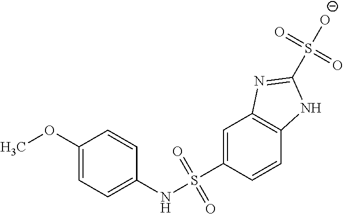 [SMILES: COc1ccc(NS(=O)(=O)c2ccc3[nH]c(S(=O)(=O)[O-])nc3c2)cc1] |
| 20 | 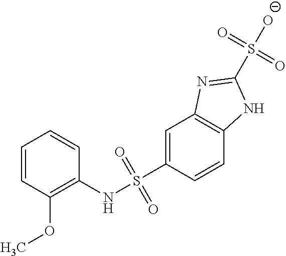 [SMILES: COc1ccccc1NS(=O)(=O)c1ccc2[nH]c(S(=O)(=O)[O-])nc2c1] |
| 21 | 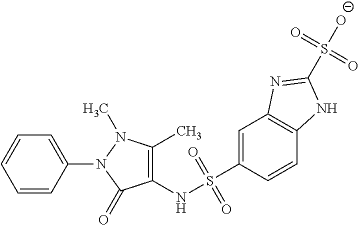 [SMILES: Cc1c(NS(=O)(=O)c2ccc3[nH]c(S(=O)(=O)[O-])nc3c2)c(=O)n(-c2ccccc2)n1C] |
| 22 | 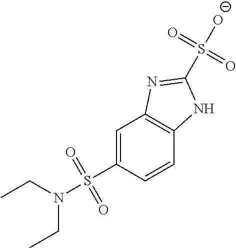 [SMILES: CCN(CC)S(=O)(=O)c1ccc2[nH]c(S(=O)(=O)[O-])nc2c1] |
| 23 | 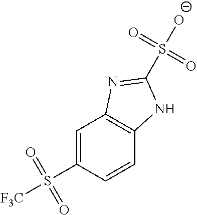 [SMILES: O=S(=O)([O-])c1nc2cc(S(=O)(=O)C(F)(F)F)ccc2[nH]1] |
| 24 | 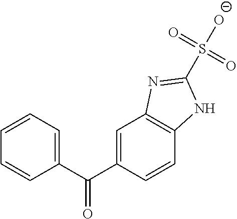 [SMILES: O=C(c1ccccc1)c1ccc2[nH]c(S(=O)(=O)[O-])nc2c1] |
| 25 | 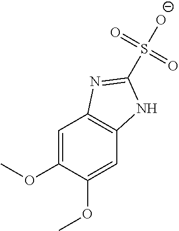 [SMILES: COc1cc2nc(S(=O)(=O)[O-])[nH]c2cc1OC] |
| 26 | 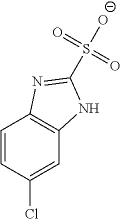 [SMILES: O=S(=O)([O-])c1nc2ccc(Cl)cc2[nH]1] |
| 27 | 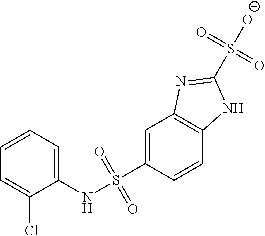 [SMILES: O=S(=O)([O-])c1nc2cc(S(=O)(=O)Nc3ccccc3Cl)ccc2[nH]1] |
| 28 | 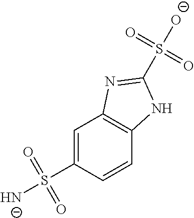 [SMILES: [NH-]S(=O)(=O)c1ccc2[nH]c(S(=O)(=O)[O-])nc2c1] |
| 29 | 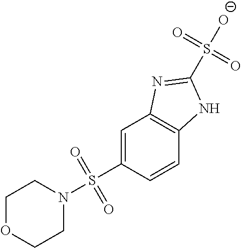 [SMILES: O=S(=O)([O-])c1nc2cc(S(=O)(=O)N3CCOCC3)ccc2[nH]1] |
| 30 | 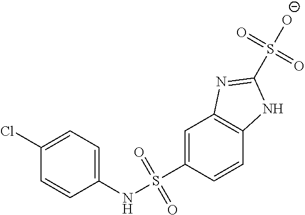 [SMILES: O=S(=O)([O-])c1nc2cc(S(=O)(=O)Nc3ccc(Cl)cc3)ccc2[nH]1] |
| 31 | 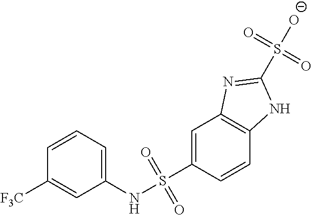 [SMILES: O=S(=O)([O-])c1nc2cc(S(=O)(=O)Nc3cccc(C(F)(F)F)c3)ccc2[nH]1] |
| 32 | 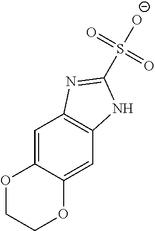 [SMILES: O=S(=O)([O-])c1nc2cc3c(cc2[nH]1)OCCO3] |
| 33 | 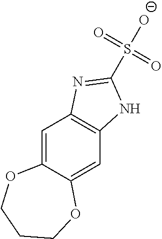 [SMILES: O=S(=O)([O-])c1nc2cc3c(cc2[nH]1)OCCCO3] |
| 34 | 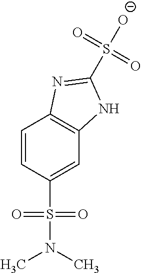 [SMILES: CN(C)S(=O)(=O)c1ccc2nc(S(=O)(=O)[O-])[nH]c2c1] |
| 35 | 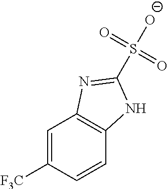 [SMILES: O=S(=O)([O-])c1nc2cc(C(F)(F)F)ccc2[nH]1] |

FERMscores for the library of interest, spanning from 0.4 to 13.7, are indicated in Table 3. The parent compound 2 scored the third-highest FERMscore. The two compounds giving higher calculated FERMscores (compounds 18 and 29) in addition to a third compound, 4, which possessed a FERMscore in the top 15% of all derivatives, were synthesized under contract by Enamine Ltd. (see Intermediate and Analog Routes below). In addition to the compounds predicted to have improved binding affinity, three immediately available compounds (15, 24, and 26) were acquired to test the predictive capacity of the employed scoring method (Tables 2 and 3).

TABLE 3

| Compound No. | FERMscore |
|---|---|
| 29 | 13.7 |
| 18 | 9.25 |
| 2 | 8.28 |
| 5 | 4.92 |
| 4 | 4.45 |
| 34 | 3.75 |
| 14 | 3.23 |
| 13 | 2.99 |
| 12 | 2.98 |
| 9 | 2.97 |
| 8 | 2.33 |
| 22 | 1.76 |
| 17 | 1.64 |
| 21 | 1.44 |
| 11 | 1.31 |
| 19 | 1.29 |
| 30 | 1.28 |
| 26 | 1.20 |
| 7 | 1.15 |
| 6 | 1.10 |
| 23 | 1.03 |
| 15 | 0.969 |
| 20 | 0.902 |
| 33 | 0.792 |
| 16 | 0.781 |
| 32 | 0.756 |
| 28 | 0.756 |
| 35 | 0.751 |
| 25 | 0.750 |
| 3 | 0.732 |
| 10 | 0.723 |
| 24 | 0.643 |
| 31 | 0.534 |
| 27 | 0.406 |

3. Intermediate Route I

In various aspects, the intermediates disclosed herein can be synthesized by the general method shown in Scheme 6 below.

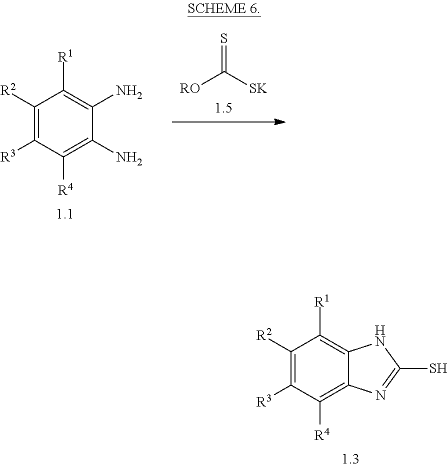

A mixture of the corresponding phenylenediamine 2.1 (0.1 mol), sodium ethyl xanthate (0.14 mol), ethanol (100 mL), water (16 mL) was refluxed under stirring for 3 h. Activated carbon was added to a slightly cooled reaction mixture (˜5 g), and the mixture was refluxed again for 10 min. Then the mixture was cooled to room temperature, the filtrate was acidified by conc. HCl (pH˜2) and left standing in a refrigerator (0-+5° C.) overnight. The precipitate formed was filtered, washed with water, and dried. Compound 2.2 was then purified by dissolving in water in the presence of alkali and re-precipitation by HCl (a typical yield for this reaction is 15%).

4. Intermediate Route II

In various aspects, the intermediates disclosed herein can be synthesized by the general method shown in Scheme 7 below.

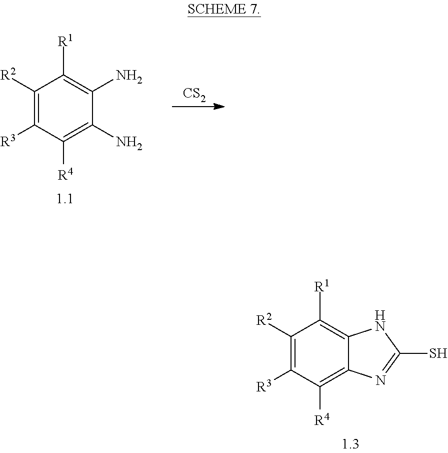

In various aspects, 1H-benzo[d]imidazole-2-thiol (1.3) was prepared as described by Mavrova et al. (Mavrova, A., et al. (2007) Bioorg. Med. Chem. 15, 6291-6297). Briefly, 1H-benzo[d]imidazole-2-thiols (1.3) were obtained by fusion of the corresponding benzimidazole (1.1) with sulfur, e.g., carbon disulfide, at 180° C. for 30 min.

5. Analog Route I

The analogs disclosed herein can be synthesized by the general method shown in Scheme 8 below.

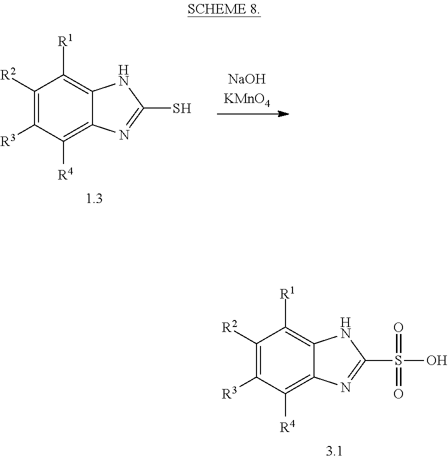

Compound 2.2 (0.04 mol) was dissolved in a solution of NaOH in water (50%, 40 mL). A solution of KMnO₄ (0.08 mol) in water (200 mL) was added to the alkaline solution under intensive stirring. Then the mixture was refluxed for 30 min before being cooled to room temperature. The precipitate was filtered off and the filtrate acidified to pH 1. The precipitate of compound 4.1 was then filtered, washed with water and dried (a typical yield for this reaction is 30%).

### a. Synthesis of 7-fluoro-2-sulfo-1H-benzo[d]imidazole-5-carboxylic acid (4)

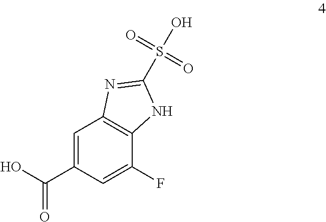 [SMILES: O=C(O)c1cc(F)c2[nH]c(S(=O)(=O)O)nc2c1]

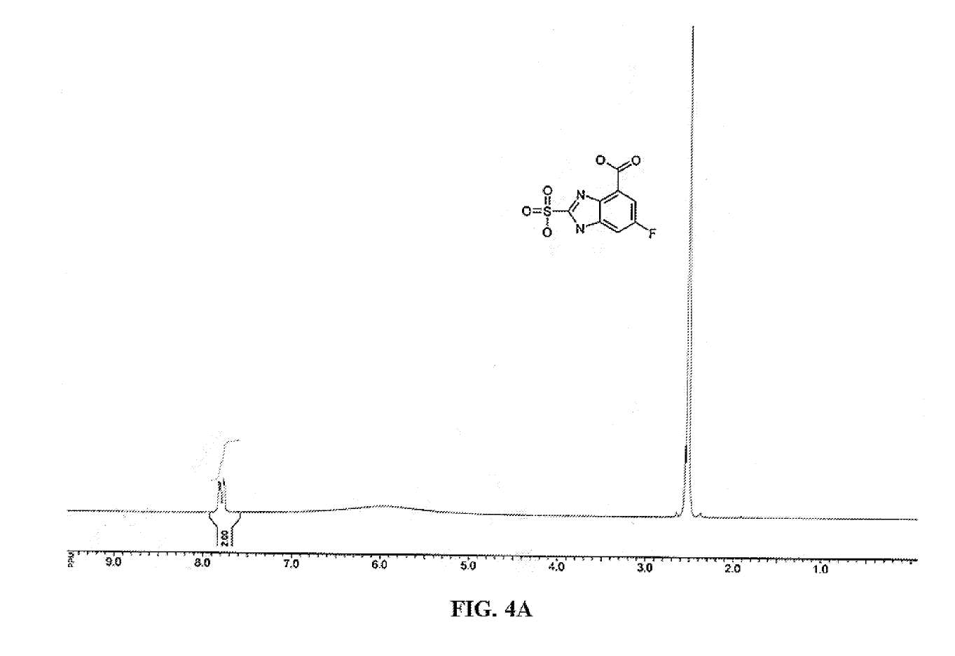

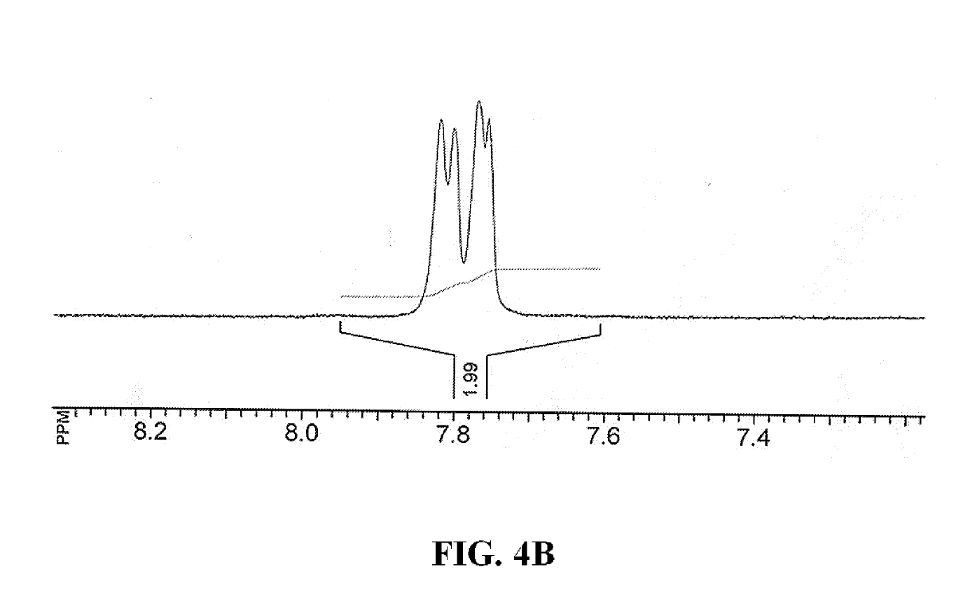

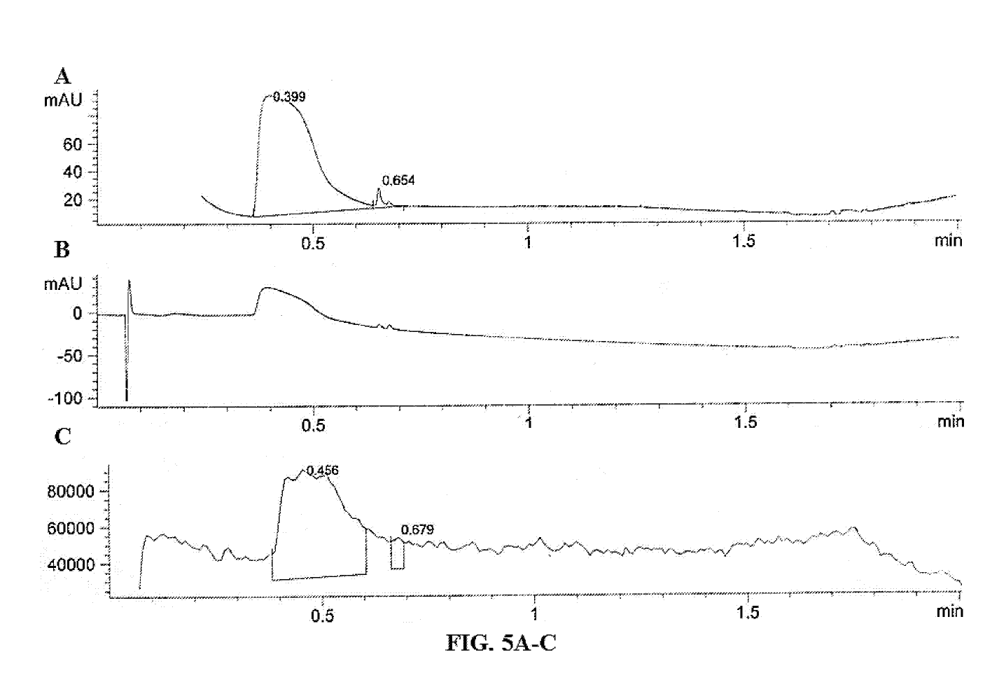

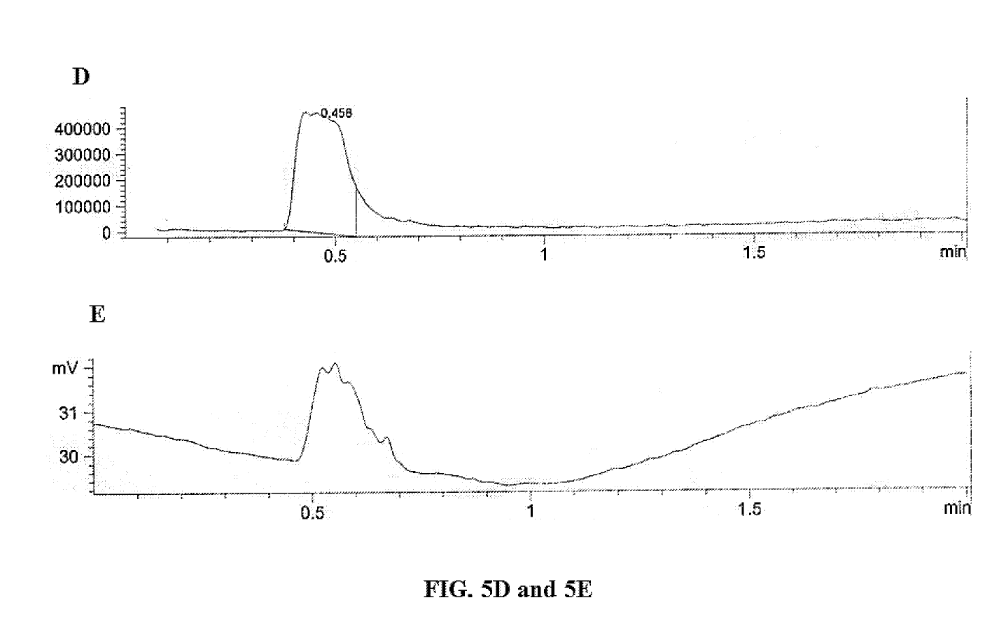

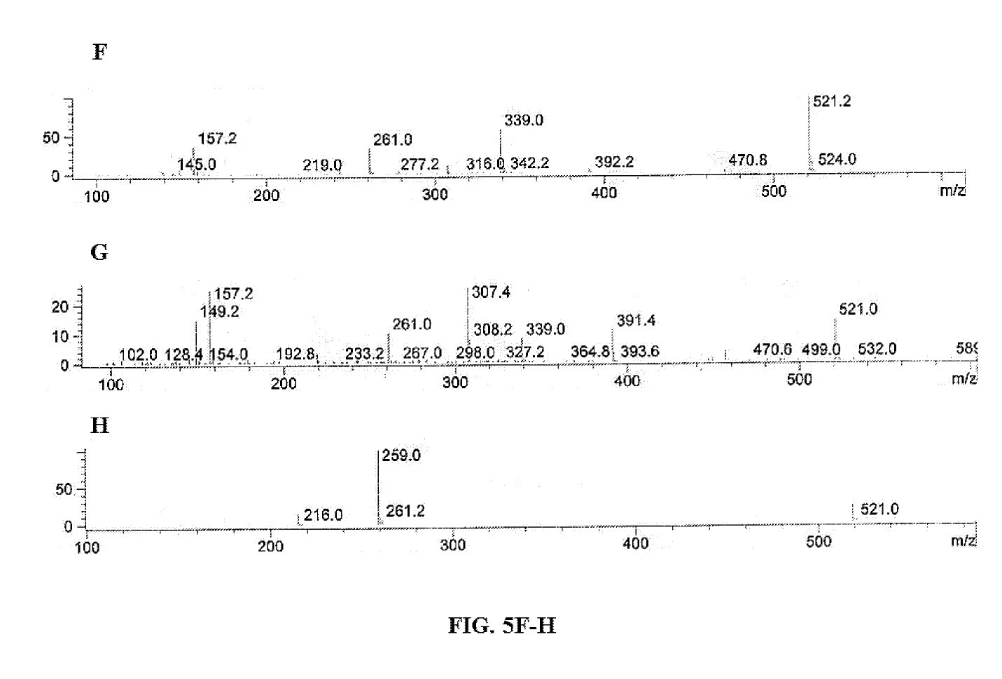

Compound 4 was prepared as described above. ¹H NMR of compound 4 is shown in FIGS. 4A and 4B. ¹H NMR (500 MHz, DMSO) δ 7.79 (2H, q). FIG. 4B is a closer perspective of the region of interest. The liquid chromatography elution profile and mass spectrometry profile are shown FIG. 5A-H.

### b. Synthesis of 5-(n-butylsulfamoyl)-1H-benzo[d]imidazole-2-sulfonic acid (18)

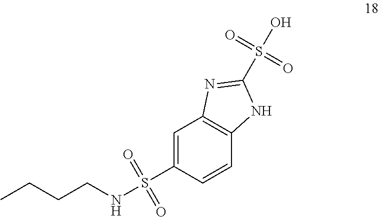 [SMILES: CCCCNS(=O)(=O)c1ccc2[nH]c(S(=O)(=O)O)nc2c1]

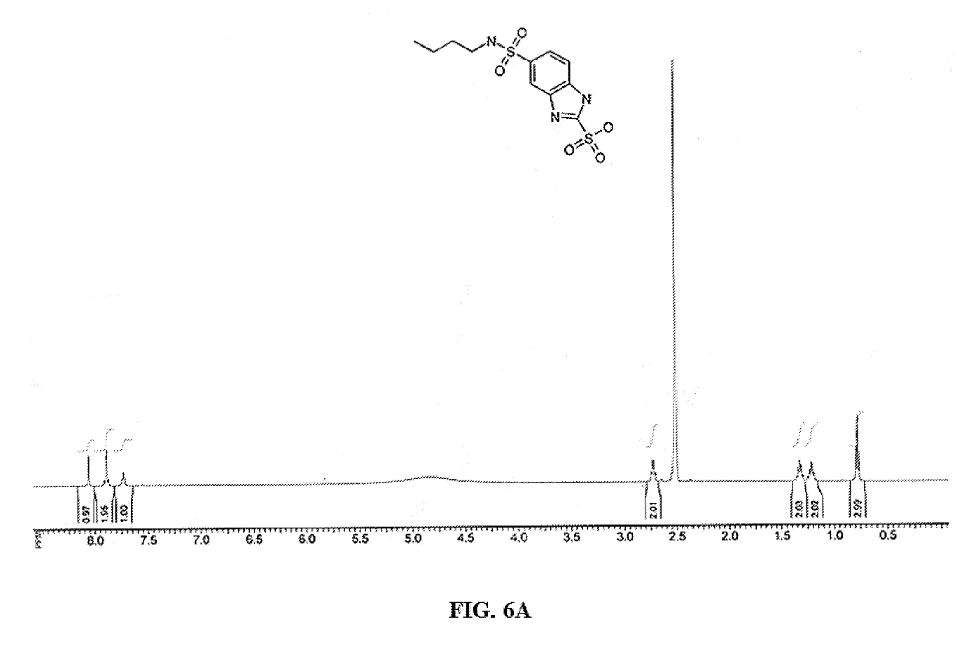

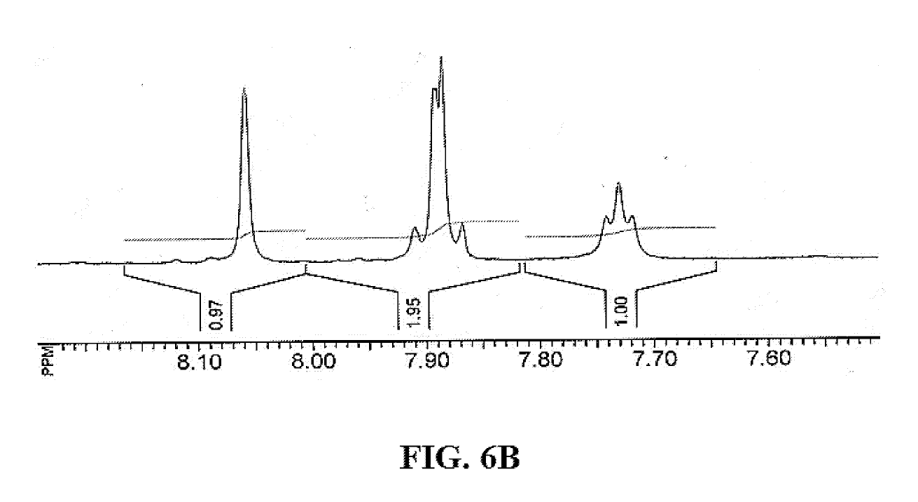

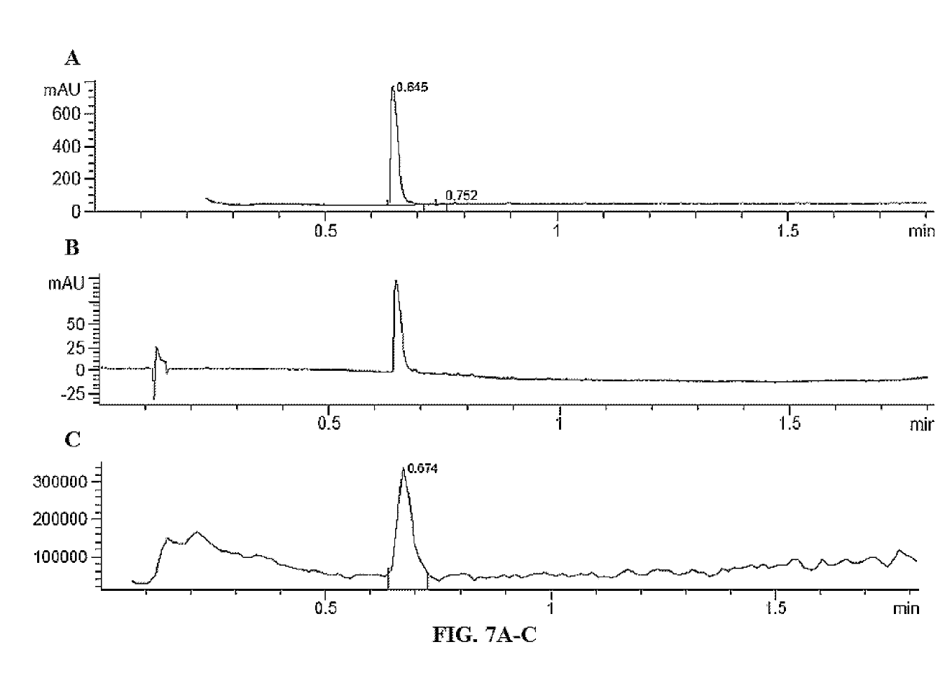

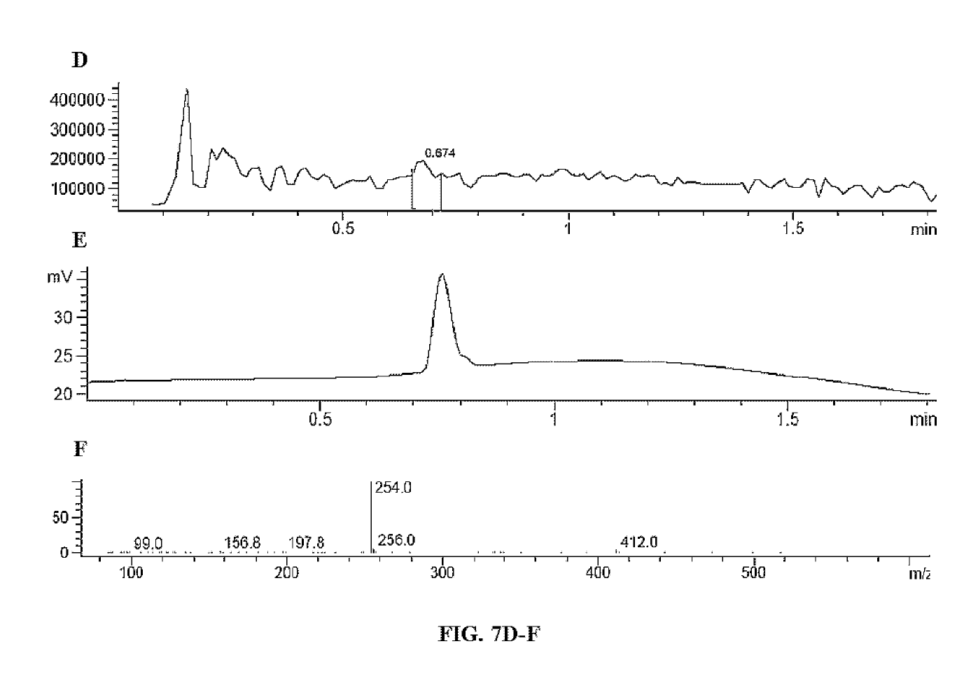

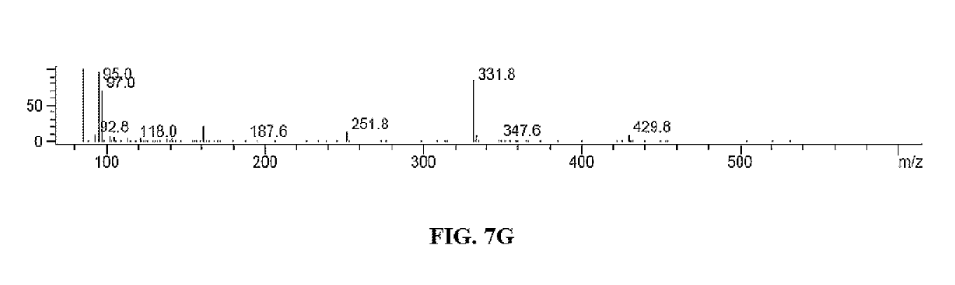

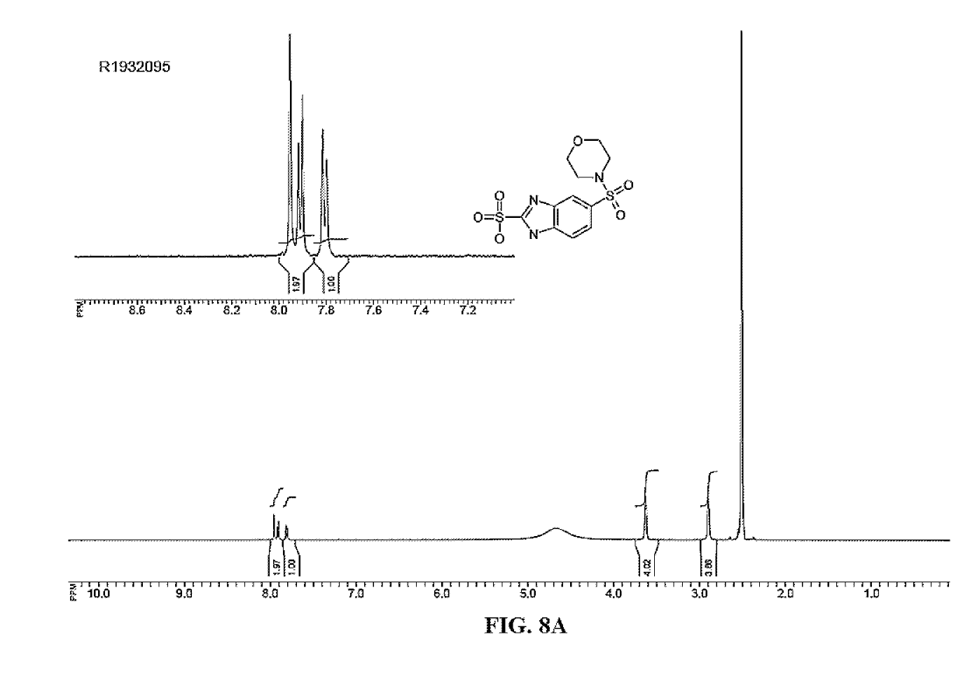

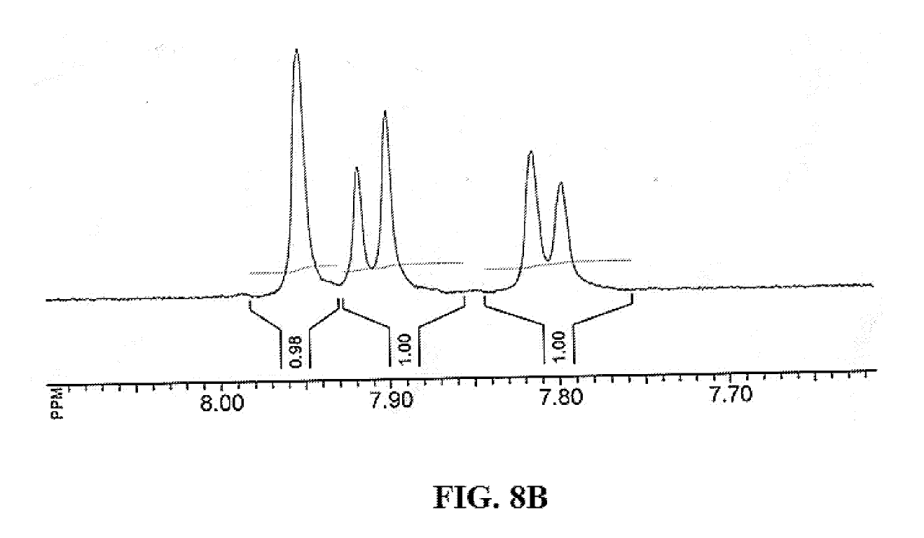

Compound 18 was prepared as described above. ¹H NMR of compound 18 is shown in FIGS. 6A and 6B. ¹H NMR (500 MHz, DMSO) δ 8.06 (1H, s), 7.89 (2H, t), 7.73 (1H, t), 2.74 (2H, q), 1.35 (2H, t), 1.23 (2H, q), 0.80 (3H, t). FIG. 8B shows a close perspective of the region of interest. The liquid chromatography elution profile and mass spectrometry profile are shown in FIG. 7A-G.

### c. Synthesis of 5-(morpholinosulfonyl)-1H-benzo[d]imidazole-2-sulfonic acid (29)

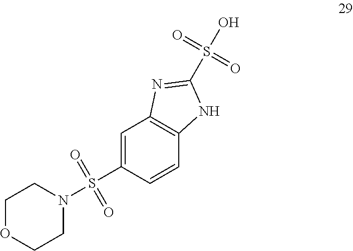 [SMILES: O=S(=O)(O)c1nc2cc(S(=O)(=O)N3CCOCC3)ccc2[nH]1]

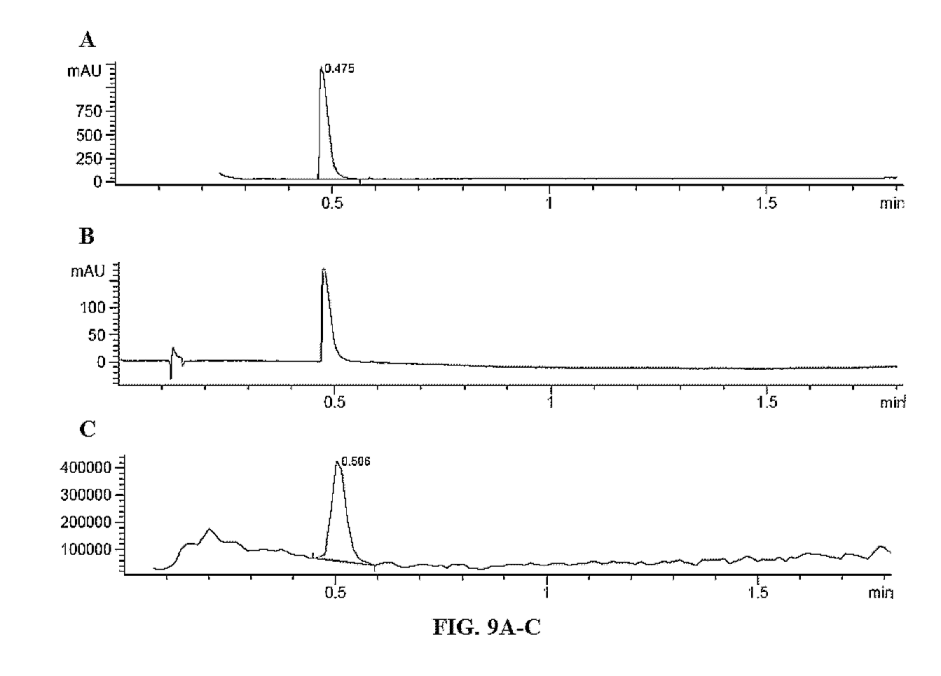

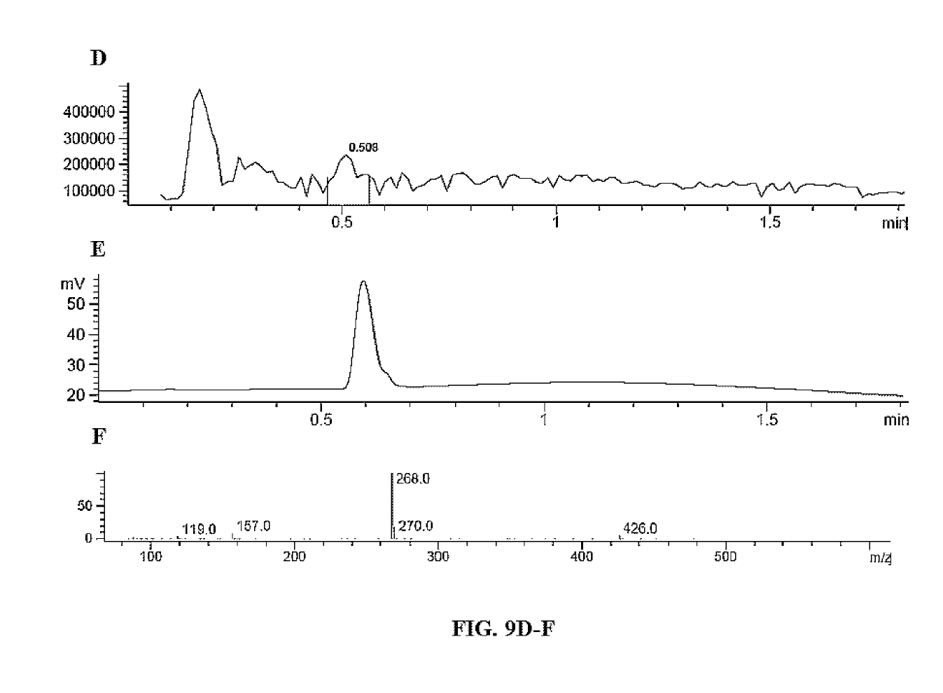

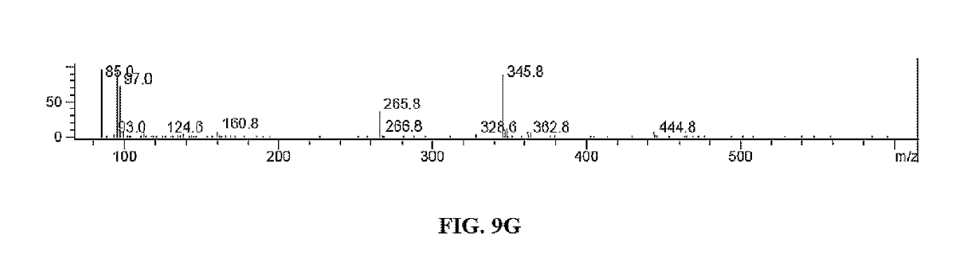

Compound 29 was prepared as described above. ¹H NMR of compound 29 is shown in FIGS. 8A and 8B. ¹H NMR (500 MHz, DMSO) δ 7.95 (1H, s), 7.91 (1H, d), 7.81 (1H, d), 3.63 (4H, s), 2.90 (4H, s). FIG. 8B shows a close perspective of the region of interest. The liquid chromatography elution profile and mass spectrometry profile are shown in FIG. 9A-G.

6. Analog Route II

The analogs disclosed herein can be synthesized by the general method shown in Scheme 9 below.

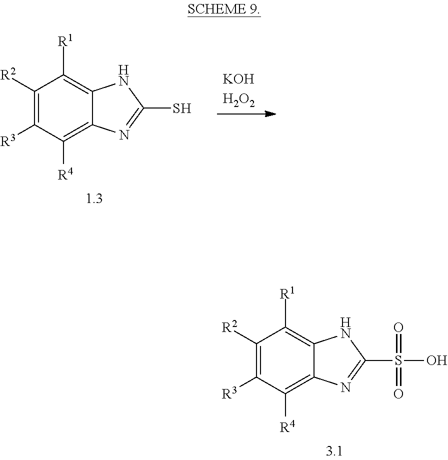

In various aspects, 1H-benzo[d]imidazole-2-sulfonic acid (4.1) was prepared as described by Mavrova et al. (Mavrova, A., et al. (2007) Bioorg. Med. Chem. 15, 6291-6297). Briefly, 1H-benzo[d]imidazole-2-thiols (1.3) were oxidized with hydrogen peroxide and potassium hydroxide, leading to the formation of 1H-benzo[d]imidazole-2-sulfonic acid (3.1).

7. Protein Expression and Purification

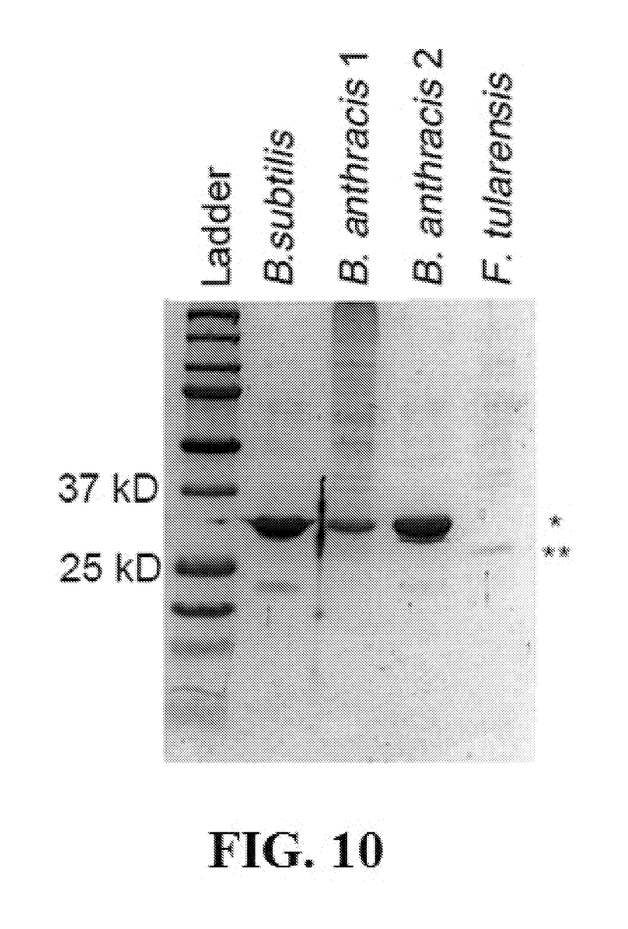

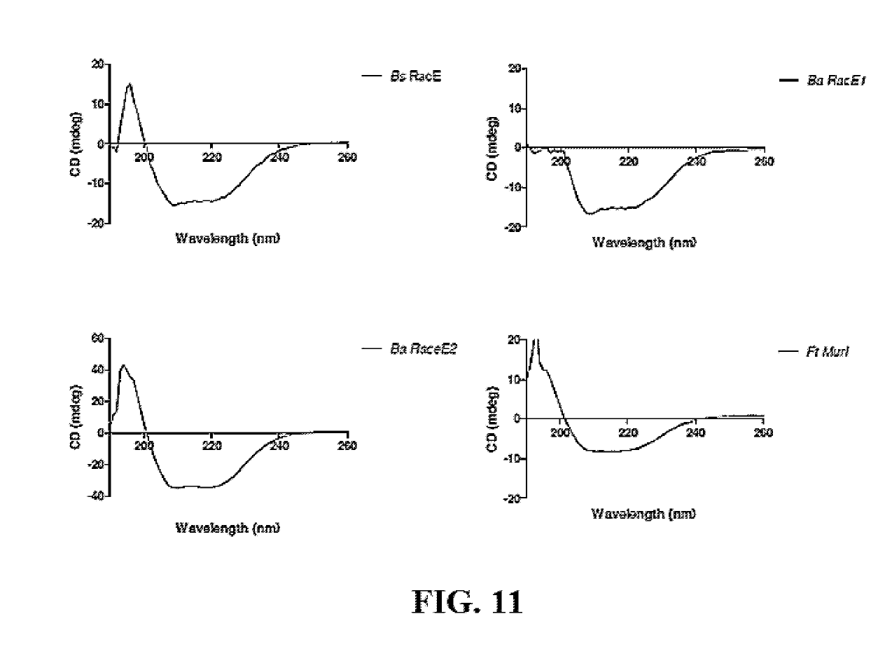

Genes of glutamate racemase were isolated from B. subtilis, B. anthracis (two isozymes), and F. tularensis, and expressed in E. coli and purified by using a protocol previously described by Whalen and co-workers (Whalen, K. L., et al. (2011) Mol. Inf. 30, 459-471). Briefly, His₆-tagged recombinant proteins were purified by a two-step process composed of cobalt-affinity (His-Select Affinity Resin, SigmaAldrich) and anion-exchange (UNO Q Continuous Bed column, BioRad) chromatography. Proteins were stored in buffer containing 100 mm NaCl, 50 mm Tris, 0.2 mm DTT, pH 8.0 at a concentration of 7-10 mg mL⁻¹. Molecular weight was confirmed by SDS-PAGE analysis (FIG. 10), and degree of protein folding was assessed by circular dichroism (CD; FIG. 11).

8. In Vitro Inhibition of Enzyme Activity

Steady-state kinetics for D-to-L racemization was measured by CD on a JASCO J-715 spectropolarimeter. All compound stocks were made up in 50 mm potassium borate buffer, pH 8.0, at concentrations in the range of 25-100 mm depending on compound solubility. Reactions were carried out at 25° C. in 50 mm potassium borate buffer, pH 8.0, with 1 mm purified enzyme. CD signal (mdeg) was measured continuously at 220 nm for 10 min. Plots of CD versus time were fit linearly to obtain initial velocity. Substrate was varied from 0.25 to 5 mm. For Kᵢ determination, three MichaelisMenten curves were obtained for each inhibitor: one in the absence of inhibitor and two in varying concentrations of inhibitor. A single data set, composed of three curves, was fit to a competitive inhibition model using GraphPad Prism v.5.0 (2007, GraphPad Software, San Diego, Calif. (USA)), and the Kᵢ was obtained as a best-fit value. For IC₅₀ determination, reactions were supplemented with varying concentrations of inhibitor, and the observed V₀ (nmol s⁻¹) was normalized to a percent activity value based on an uninhibited reaction. Percent activity values were plotted versus the log of the inhibitor concentration. The data set was then fit to a log [inhibitor] versus response model (with variable slope) to calculate the IC₅₀, using GraphPad Prism v.5.0 (2007, GraphPad Software, San Diego, Calif. (USA)). For LiPE calculations, compound log P values for the ionic species were calculated with MarvinSketch (ChemAxon).

9. In Vivo Inhibition of Bacterial Growth

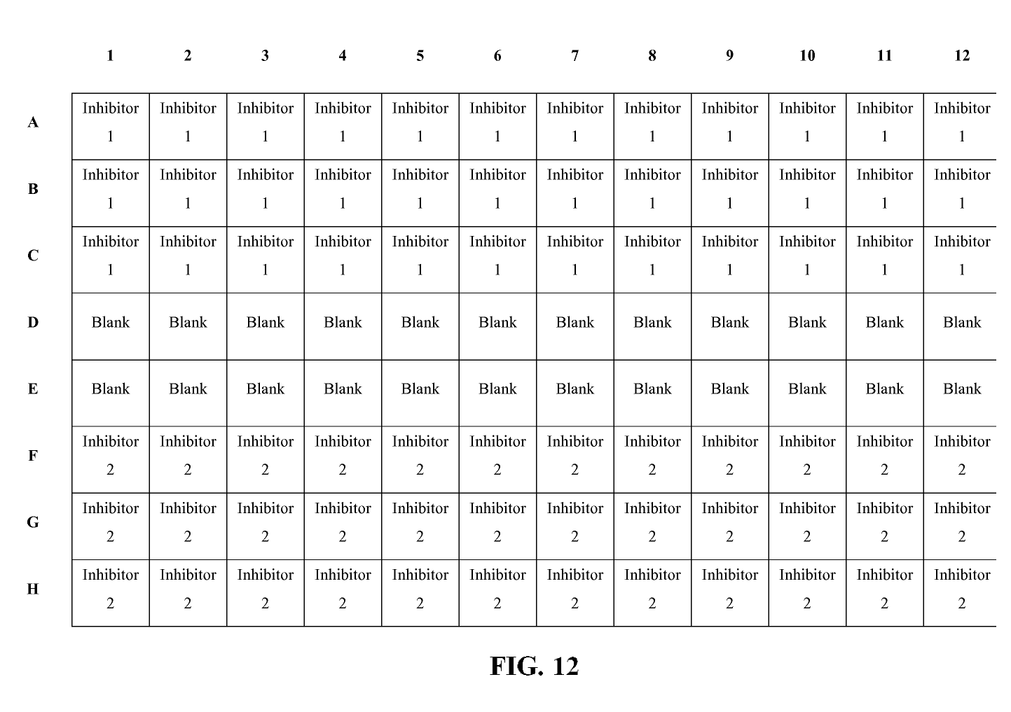

A 5 mL culture of bacteria (B. subtilis DB104, E. coli Acella, or S. aureus ATCC 12600) was incubated overnight at 37° C. in tryptic soy broth one day prior to assay; 96-well plates were prepared with 2× media, phosphate-buffered saline, the compound of interest, and a 20 mL inoculum of bacteria, totaling 200 mL per well. Compound stocks were prepared in phosphate-buffered saline at a concentration of 10, 12.5, or 25 mm, depending on compound solubility. A serial dilution ranging from 0.1 to 3000 mm for the compound of interest was assayed. The overnight culture, at an optical density of ˜2.0, was diluted 20-fold in water prior to inoculation, such that initial optical densities were ˜0.01. A table of reagent volumes and diagram of plate layout can be found in Table 4 and FIG. 12. Plates were mixed and incubated at 37° C. for 24 h. Absorbance at 2, 600 nm was measured on a GloMax-Multi Detection System. MIC₅₀ values were determined by fitting data to a log [inhibitor] versus response model using GraphPad Prism v.5.0 (2007, GraphPad Software, San Diego, Calif. (USA)). The bottom and top values were constrained to 20 and 100%, respectively.

TABLE 4
Inhibitor X with 10 mM and 0.1 mM Stocks

| | Com- | Com- | | | | | |
|---|---|---|---|---|---|---|---|
| Media | pound | pound | [Stock] | Inoculum | PBS | Final | Col- |
| (μL) | (μM) | (μL) | (μM) | (μL) | (μL) | Vol. | umn |
| 100 | 0 | 0 | 100 | 20 | 80 | 200 | 1 |
| 100 | 0.1 | 0.2 | 100 | 20 | 79.8 | 200 | 2 |
| 100 | 1 | 2 | 100 | 20 | 78 | 200 | 3 |
| 100 | 10 | 20 | 100 | 20 | 60 | 200 | 4 |
| 100 | 50 | 1 | 10000 | 20 | 79 | 200 | 5 |
| 100 | 100 | 2 | 10000 | 20 | 78 | 200 | 6 |
| 100 | 500 | 10 | 10000 | 20 | 70 | 200 | 7 |
| 100 | 1000 | 20 | 10000 | 20 | 60 | 200 | 8 |
| 100 | 1500 | 30 | 10000 | 20 | 50 | 200 | 9 |
| 100 | 2000 | 40 | 10000 | 20 | 40 | 200 | 10 |
| 100 | 2500 | 50 | 10000 | 20 | 30 | 200 | 11 |
| 100 | 3000 | 60 | 10000 | 20 | 20 | 200 | 12 |

10. Cell Wall Lysis Assay

Bacterial cell wall lysis was assayed using a modification of the CytoTox-Glo assay (Niles, A. L., et al. (2007) Anal. Biochem. 366, 197-206) (Promega): 100 mL of the contents of a 96-well plate treated as described above were moved to a white, round bottom, 96-well plate. CytoTox-Glo reagent buffer (25 mL) was added to each well, mixed and incubated at room temperature in the dark for 15 min. Luminescence was measured on a GloMax-Multi Detection System Luminescent values were normalized based on the absorbance value at 2, 600 nm for each respective well. Data was then fit to a log [agonist] versus response model, with no upper or lower constraints, using GraphPad Prism v5.0 (2007, GraphPad Software, San Diego, Calif. (USA)).

11. Antimicrobial Data and the Glutamate Racemase

The function and structure of glutamate racemase enzyme is known in the art as is described by Whalen, K. et al. Molecular Informatics, 2011, 30, 459-471, which is hereby incorporated by reference in its entirety. The figures and data described below, that pertain to in vitro experiments, were obtained using the general methods described by Whalen, K. et al. Molecular Informatics, 2011, 30, 459-471. Figures and data pertaining to the acquisition of MIC₅₀ values for inhibition of bacterial growth and EC₅₀ values for bacterial lysis were acquired as described generally in Motyl, M. et al. Current Protocols in Pharmacology, 2006, 13A.3 and Niles, A. et al. Analytical Biochemistry, 2007, 366, 197-206, respectively.

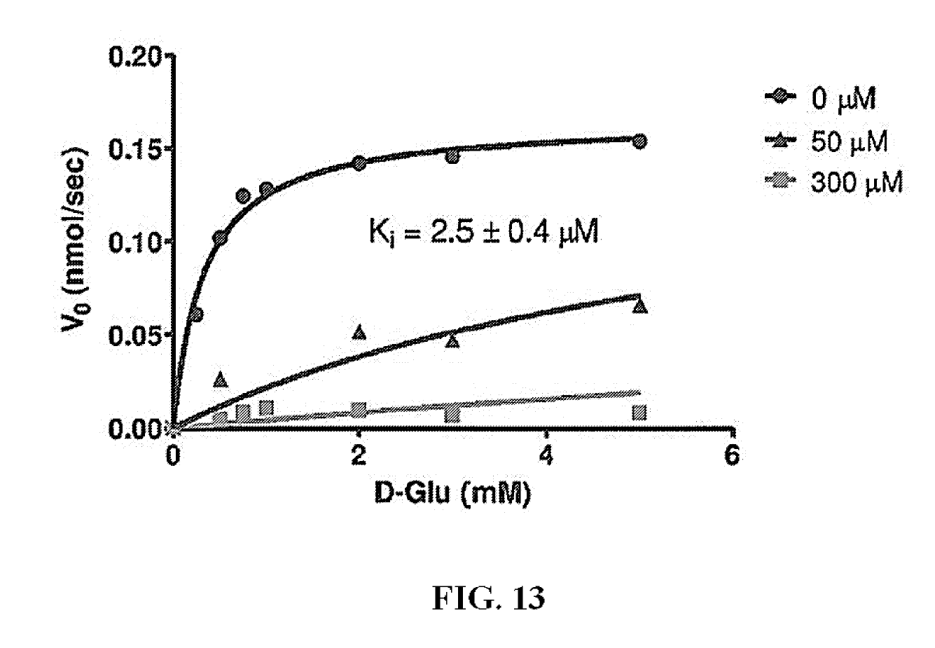

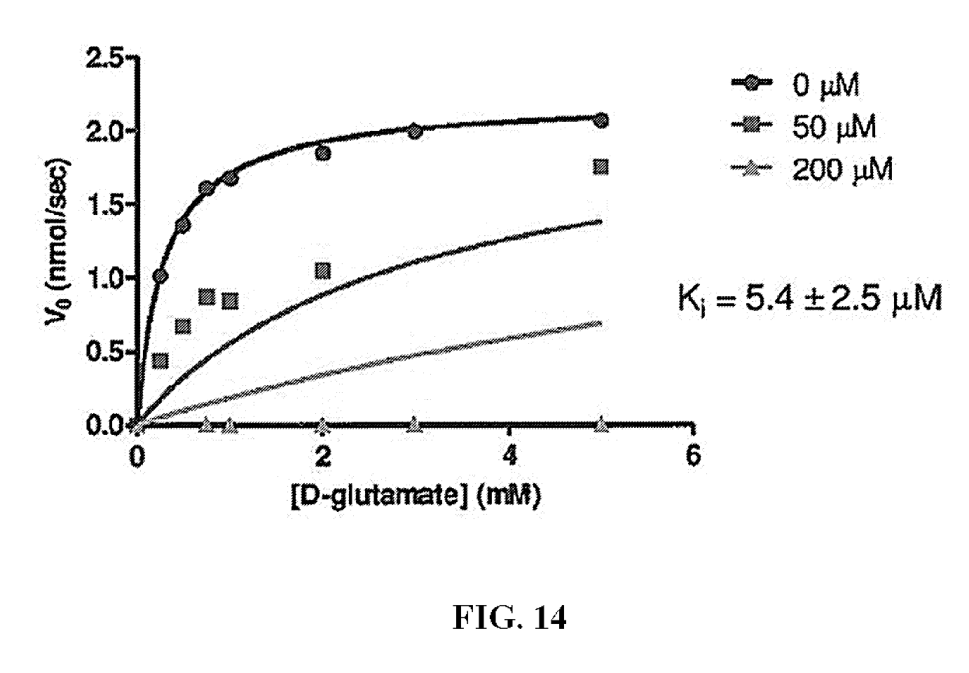

Compound 4 was tested for its antimicrobial activity. Glutamate racemase of Bacillus subtilis and Bacillus anthracis can be inhibited by compound 4. FIG. 13 shows in vitro data for the inhibition of glutamate racemase from Bacillus subtilis by compound 4. The data shows that compound 4 effectively inhibits glutamate racemase from Bacillus subtilis. FIG. 14 shows in vitro data for the inhibition of glutamate racemase isozyme 2 from Bacillus anthracis (BsRaceE2) by compound 4. FIG. 13 and FIG. 14 indicate that the glutamate racemase of both species of Bacillus are effectively inhibited by compound 4.

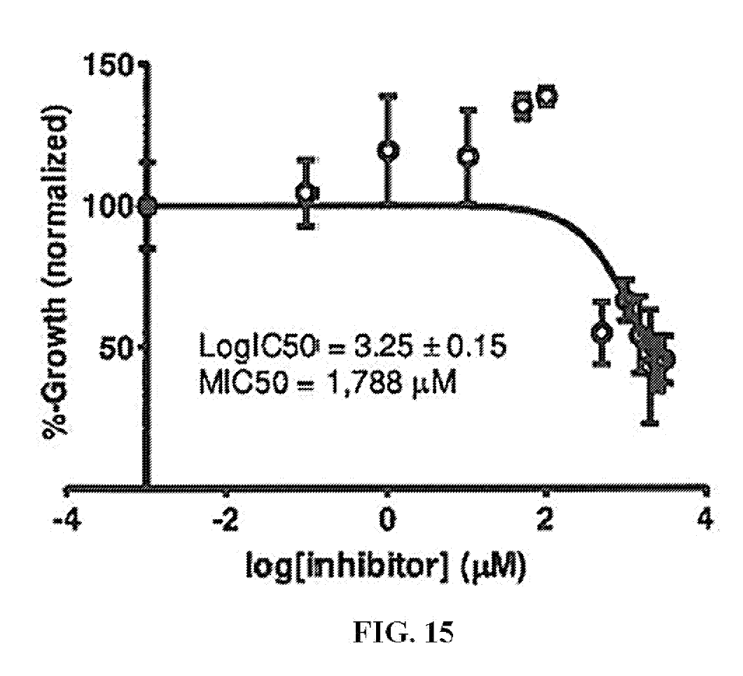

The inhibition of glutamate racemase of Bacillus subtilis by compound 1 provides inhibition of the growth of Bacillus subtilis. FIG. 15 shows that inhibition of growth of Bacillus subtilis after treatment of compound 4.

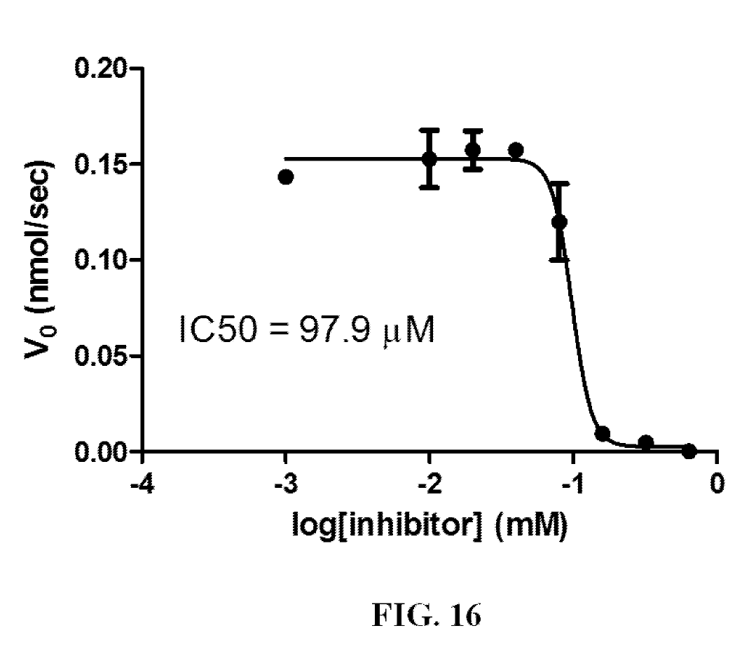

Compound 4 was additionally shown to inhibit glutamate racemase from Helicobacter pylori. H. pylori is a gram-negative microbe which has been estimated to inhabit the upper gastrointestinal tract of 50% of the world's population. Chronic H. pylori infections have been shown to lead to duodenal ulcers as well as stomach cancer. Compound 4, as shown in FIG. 16, is an inhibitor of glutamate racemase from Helicobacter pylori. Table 5 shows best-fit values, standard error and IC₅₀ values for compound 4.

TABLE 5

| | |
|---|---|
| Best-Fit Values | Standard Error |

| | | | |
|---|---|---|---|
| LogIC₅₀ | −0.9935 | LogIC₅₀ | 0.01401 |
| Hill Slope | −6.498 | Hill Slope | 0.7652 |
| IC₅₀ (mM) | 0.1015 | | |

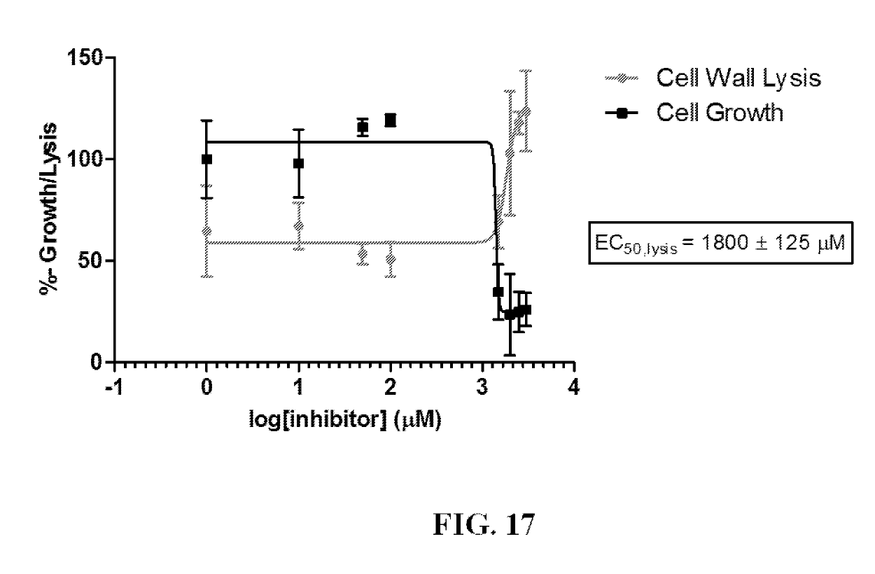

FIG. 17 indicates that compound 4 has the ability to promote bacterial cell wall lysis. Utilizing a proprietary kit advertised for the monitoring of cell wall integrity in mammalian cells (CytoTox-Glo, Promega Corp.), a dose-dependent increase is seen in the leakage of intracellular proteases when B. subtilis cells are exposed to compound 4, see FIG. 17. This indicates that compound 4 is operating “on-target” and that microbial growth inhibition is a result of cell wall peptidoglycan disruption.

12. In Vitro Testing of Derivatives

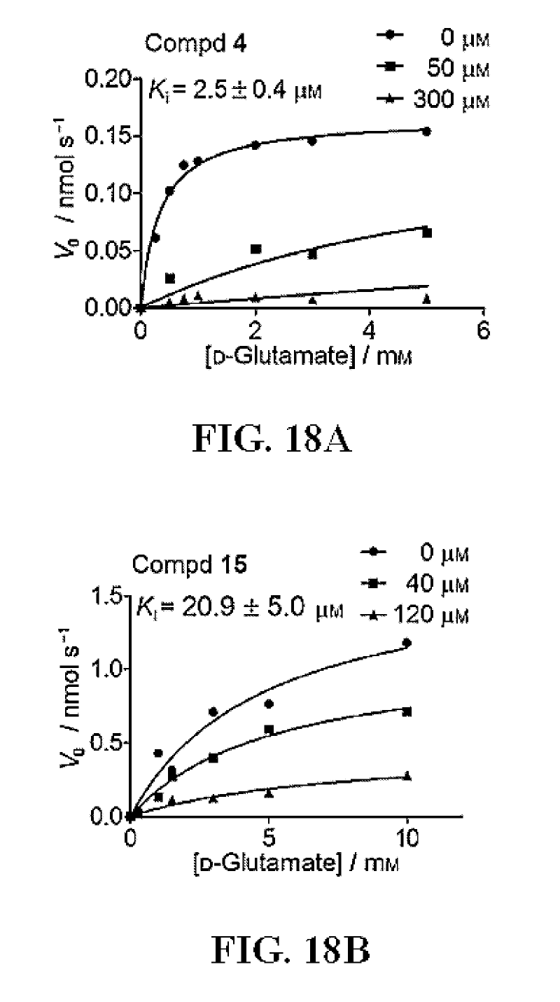

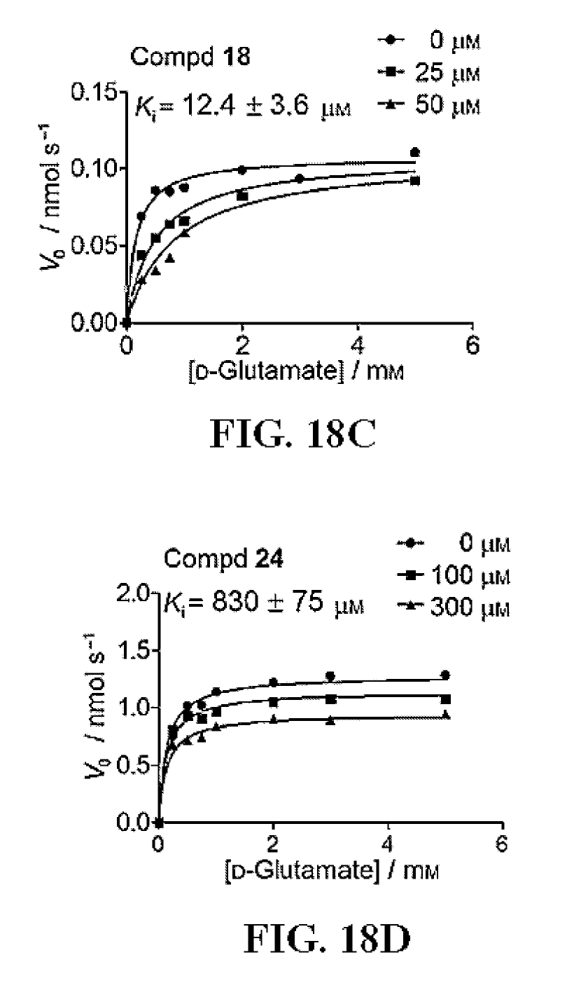

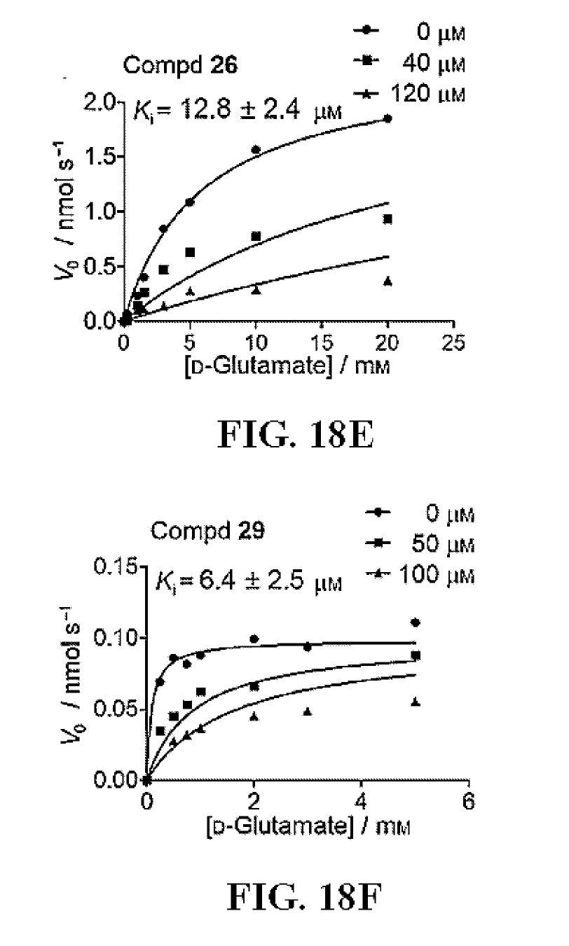

Inhibition constants (Kᵢ) were acquired for all derivatives against purified GR from B. subtilis (FIG. 18A-F, Table 6). Compounds 15, 24, and 26, all possessing predicted FERMscores lower than the parent compound, gave Kᵢ values greater than or within error of that of the parent compound. Compound 24 suffered from a nearly 100-fold loss in binding affinity, which was well predicted by FERM-SMD, as it possessed the lowest FERMscore of the compounds tested. Of the compounds predicted to be higher-affinity binders by FERMscore, compounds 18 and 29 have Kᵢ values within error of the parent compound, although the Kᵢ value of 29 is improved: 6.4 versus 9 μm. This result was not surprising considering the FERM scores only vary by 5.4 units between the parent scaffold and the highest-scoring derivative. Compound 4 was also predicted to have high affinity, and shows four-fold improved affinity over the parent compound, with a Kᵢ value of 2.5 μm. This is the most potent non-glutamate-based competitive inhibitor of GR to date. Overall, FERM-SMD was successful in distinguishing between tight binding derivatives (Kᵢ ranging from 2.5 to 12 μm) and weaker binding derivatives (Kᵢ between 13 and 830 μm).

TABLE 6

| Compound No. | Kᵢ (μM) | LE (kcal mol⁻¹ atom⁻¹)^{a} | LiPE^{b} |
|---|---|---|---|
| 2 | 9.0 ± 2.0 | 0.53 | 5.3 |
| 4 | 2.5 ± 0.4 | 0.45 | 6.0 |
| 15 | 21 ± 5.0 | 0.46 | 4.4 |
| 18 | 12 ± 3.6 | 0.30 | 5.0 |
| 24 | 830 ± 75 | 0.20 | 1.9 |
| 26 | 13 ± 2.4 | 0.48 | 4.5 |
| 29 | 6.4 ± 3.5 | 0.34 | 6.6 |

^{a}Ligand efficiencies determined by converting Kᵢ values into binding energies and dividing by the number of non-hydrogen atoms;
^{b}Lipophilic efficiencies determined by subtracting logP values from the log(Kᵢ) for each compound.

Ligand and lipophilic efficiencies (LE and LiPE) were calculated for each derivative (Table 5). LE is a way to describe normalized binding affinities for compounds of differing molecular weights (Hopkins, A. L., et al. (2004) Drug Discovery Today 9, 430-431; Hann, M. (2011) Med. Chem. Commun. 2, 349-355). Several studies have also shown that fragment-based drug discovery is more successful if high LE is maintained through lead optimization (Hopkins, A. L., et al. (2004) Drug Discovery Today 9, 430-431; Hann, M. (2011) Med. Chem. Commun. 2, 349-355). This practice lowers the occurrence of so-called “molecular obesity” as compounds are modified to achieve higher potency and favorable pharmacokinetic/pharmacodynamic profiles (Hann, M. (2011) Med. Chem. Commun. 2, 349-355). With the exception of compound 15, each assayed derivative maintained high ligand efficiency (LE>0.3 kcal mol⁻¹ atom⁻¹). Of the compounds predicted to be high affinity by the FERM-SMD method, compounds 4 and 29 exhibited higher efficiency than compound 18 (0.45, 0.34, and 0.30, respectively). Lipophilic efficiency is another measure that is indicative of successful passage down the drug development pipeline, in which affinity values are normalized for the partition coefficient (log P) of the inhibitor (Springthorpe, B. (2007) Nat. Rev. Drug Discovery 6, 881-890). Compounds 4 and 29 benefit from an improved LiPE (6.0 and 6.6, respectively) over the parent scaffold (5.3). LE and LiPE values equal to or greater than 0.3 kcal mol⁻¹ atom⁻¹ and 6.0, respectively, are in the desirable range for further study and optimization (Hopkins, A. L., et al. (2004) Drug Discovery Today 9, 430-431; Leeson, P. D., Springthorpe, B. (2007) Nat. Rev. Drug Discovery 6, 881-890).

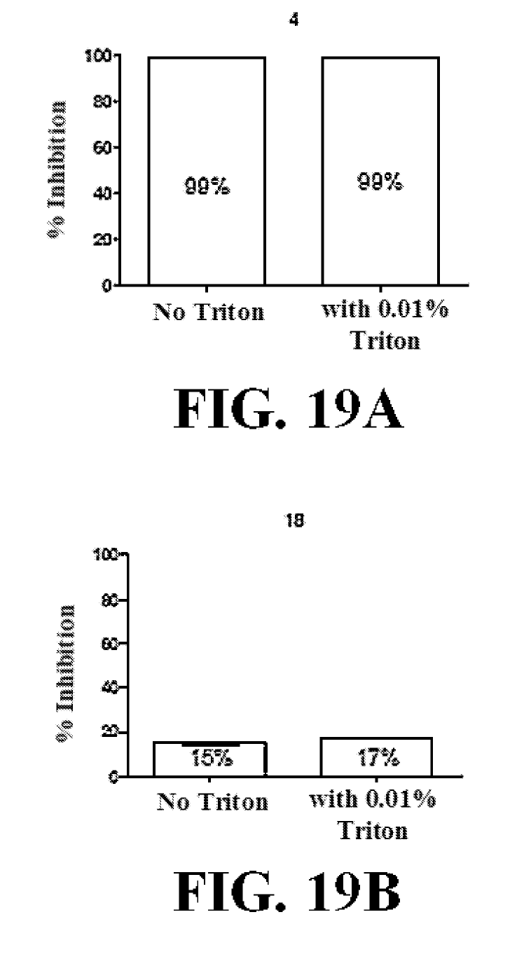

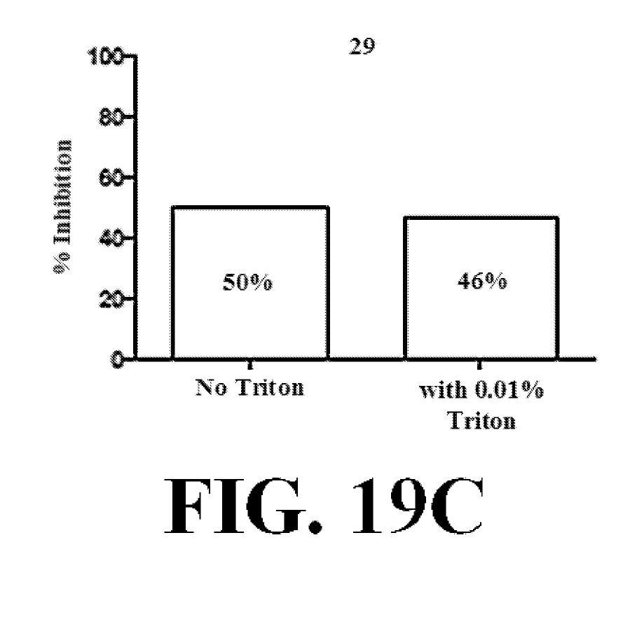

Novel compounds were also tested for the formation of colloidal aggregates, a common cause of false-positive results. Previous studies have revealed that GR is susceptible to inhibition by colloidal aggregates in a non-drug-like fashion (Whalen et al. (2009) ACS Med. Chem. Lett. 1, 9-13). To distinguish between inhibition via colloidal aggregation and true binding, enzyme activity was measured in the presence of the inhibitor in question, as well as a sub-micellular concentration of detergent, 0.01% Triton X-100. In the event that a given compound inhibits an enzyme by colloidal aggregation, the apparent inhibition will be completely relieved in the presence of detergent. Colloidal aggregates must be abandoned due to their non-drug-like mechanism. All novel compounds tested were shown not to operate through a colloidal aggregate mechanism (FIG. 19A-C).

13. In Vivo Testing of Biological Activity

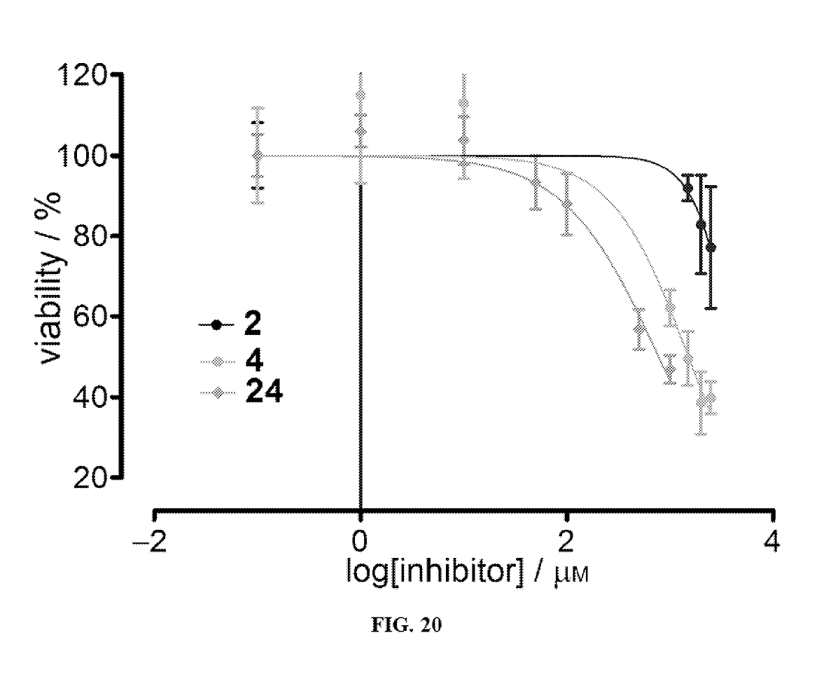

To assess the capacity of these compounds to reach the enzyme target in vivo, inhibition of bacterial growth as well as capacity to induce cell lysis was assayed with several species of bacteria. B. subtilis was investigated, as the isozyme of GR from this species was the model for all in silico predictions. Additionally, E. coli and S. aureus were investigated, as each provides a unique challenge for inhibitor compounds: an additional physical barrier to entry in the case of Gram-negative E. coli, and an abundance of efflux pumps in the case of S. aureus (Kuroda, M., et al. (2001) Lancet 357, 1225-1240). All tested derivatives of compound 2 showed increased potency with regards to growth inhibition of B. subtilis (FIG. 20). MIC₅₀ values were increased two to threefold over the parent scaffold (Table 7). Surprisingly, the least potent compound in vitro, 24, showed the greatest potency in vivo. Without wishing to be bound by theory, this may suggest that factors other than enzyme binding affinity may complicate the overall efficacy of this chemotype of antimicrobial compounds.

TABLE 7

| Compound | MIC₅₀ (mg mL⁻¹) | | |
|---|---|---|---|
| No. | B. subtilis | E. coli | S. aureus |
| 2 | 0.72 ± 0.06 | — | — |
| 4 | 0.26 ± 0.11 | 1.6 ± 0.20 | 1.0 ± 0.25 |
| 15 | >3 | — | — |
| 18 | 0.36 ± 0.02 | >3 | >3 |
| 24 | 0.14 ± 0.02 | — | — |
| 26 | >3 | — | — |
| 29 | 0.32 ± 0.01 | >3 | >3 |

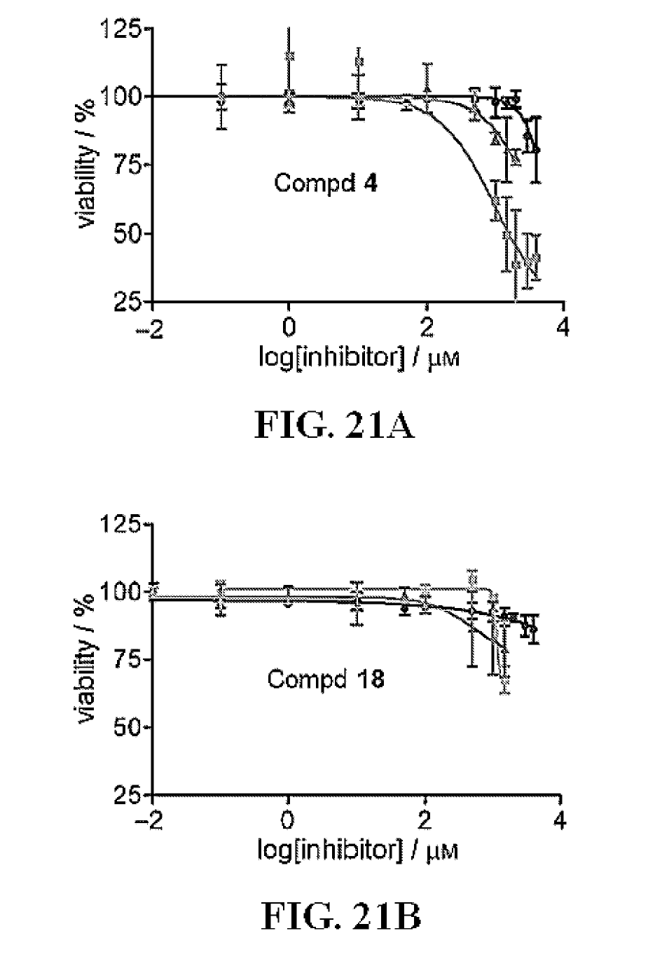

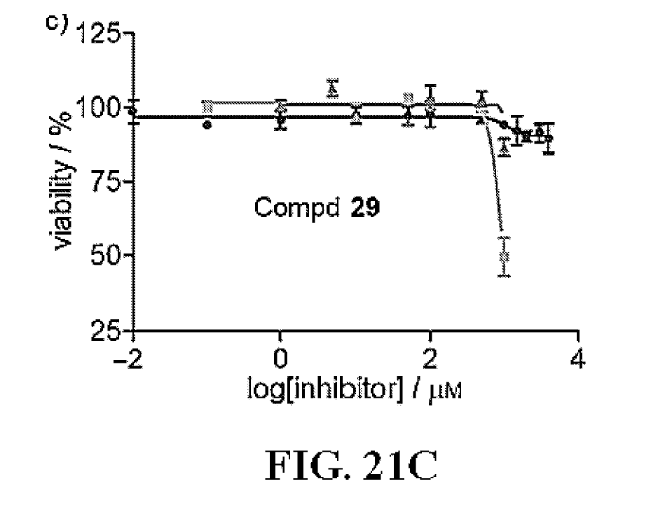

Compounds 4, 18, and 29 were also tested against E. coli and S. aureus (FIG. 21A-C). Compounds 18 and 29 were both highly specific for B. subtilis, showing no significant growth inhibition at concentrations<3 mg mL⁻¹ for E. coli and S. aureus (Table 6). In contrast, compound 4 shows growth inhibition of both E. coli and S. aureus at concentrations approximately twofold higher than the MIC₅₀ value against B. subtilis (Table 6). Examination of their respective chemical structures yields one possible rationale for this distinction (see Table 2 for structures). Compound 4 possesses a more compact structure, most likely making contacts specific to the most buried and most highly conserved region of the GR active site. Compounds 18 and 29 contain bulkier chemical additions to the benzene ring that may clash with the outer region of the GR active site, which is a more structurally diverse region. These in vitro (and in silico) results support the fragment-based strategy of growing the scaffold out of the highly buried active site without sacrificing the original contacts.

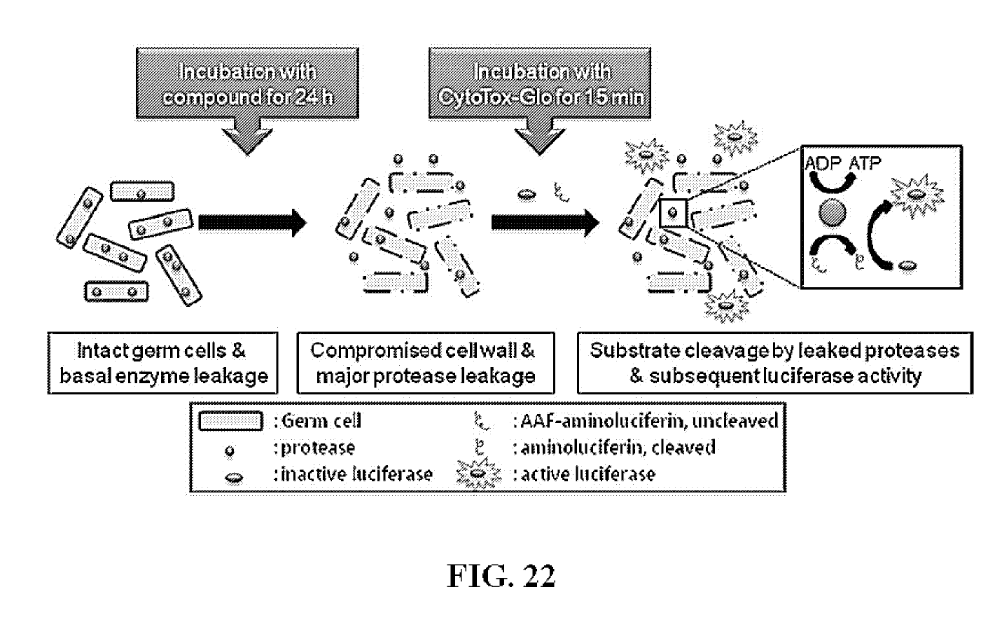

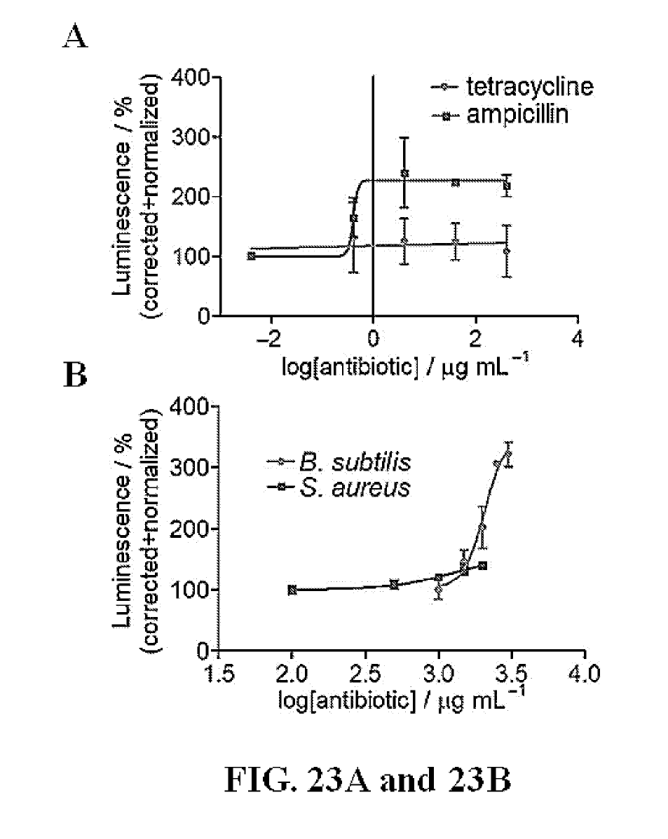

To determine the mechanism of action of the derivatives in this study, a commercially available cytotoxicity assay, CytoTox-Glo (Promega), marketed for use with mammalian cells, was adapted for use with the examined bacterial species. The relationship between cytotoxicity—specifically cell lysis—and the readout (luminescence) is outlined in FIG. 22. If the compounds reach the GR target and inhibit the production of D-glutamate, the lack of this key component results in an overall breakdown in peptidoglycan synthesis, and subsequent cell lysis caused by osmotic stress. Lysed cells then leak intracellular proteases into the surrounding media. The CytoTox-Glo reagent is composed of a pro-luciferin substrate, which once cleaved by proteases, can be acted upon by a supplied luciferase to produce the luminescent readout. Controls using USFDA-approved antibiotics with known mechanisms of action were conducted to optimize the provided reagents (FIG. 23A). S. aureus was exposed to varying concentrations of ampicillin (a transpeptidase inhibitor) or tetracycline (a microbial ribosome inhibitor) for 24 h, and then incubated with the CytoTox-Glo reagent. As expected, ampicillin yields a dose-dependent increase in luminescence, while tetracycline elicits no increase in luminescence at concentrations up to 100-fold the published MIC₅₀ value (FIG. 23A). This optimized assay was then applied to cells treated with the disclosed inhibitors.

The modified CytoTox-Glo assay confirmed 4 as acting via an inhibitory mechanism that affects the peptidoglycan with both S. aureus and B. subtilis (FIG. 23B). Luminescence increases concurrently with increased dosing of compound 4 for S. aureus. Although the assayed concentrations do not span the entire MIC₅₀ range owing to solubility limitations, an approximate 40% increase in luminescence was observed in the millimolar inhibitor concentration range. For B. subtilis, the species for which compound 4 elicits greater growth inhibition, a dose-dependent increase of 400% in luminescence in the low-millimolar range was observed. The observed EC₅₀ for lysis occurs at 520 μg mL⁻¹, a concentration only slightly above that of the MIC₅₀ value for growth inhibition (260 μg mL⁻¹). Without wishing to be bound by theory, the proximity of these two values supports cell lysis as the main cause of cell death.

14. Characterization of Exemplary Compounds

Table 8 below lists the predicted binding energy of specific compounds towards E. coli from the experimentally determined crystal structure from strain K12 (PDB accession code 2JFN). The binding energy was determined using the VINA scoring function from implementation of AutoDockVINA.

TABLE 8

| VINA | Compound | VINA Binding |
|---|---|---|
| Ranking | No. | Energy (kcal/mol) |
| 1 | 24 | 9.477 |
| 2 | 19 | 8.975 |
| 3 | 21 | 8.939 |
| 4 | 3 | 8.896 |
| 5 | 31 | 8.689 |
| 6 | 20 | 8.675 |
| 7 | 7 | 8.615 |
| 8 | 10 | 8.599 |
| 9 | 35 | 8.362 |
| 10 | 27 | 8.28 |
| 11 | 23 | 8.194 |
| 12 | 5 | 8.149 |
| 13 | 6 | 8.133 |
| 14 | 17 | 8.114 |
| 15 | 9 | 8.058 |
| 16 | 32 | 8.015 |
| 17 | 30 | 7.985 |
| 18 | 26 | 7.784 |
| 19 | 16 | 7.77 |
| 20 | 8 | 7.726 |
| 21 | 28 | 7.711 |
| 22 | 14 | 7.71 |
| 23 | 15 | 7.637 |
| 24 | 33 | 7.629 |
| 25 | 11 | 7.586 |
| 26 | 12 | 7.567 |
| 27 | 2 | 7.411 |
| 28 | 13 | 7.379 |
| 29 | 25 | 7.333 |
| 30 | 4 | 7.306 |
| 31 | 34 | 7.237 |
| 32 | 29 | 7.019 |
| 33 | 22 | 6.926 |
| 34 | 18 | 6.474 |

Table 9 below lists the predicted binding energy of specific compounds towards S. aureus from the experimentally determined crystal structure from strain MRSA252 (PDB accession code 2JFQ). The binding energy was determined using the VINA scoring function from implementation of AutoDockVINA.

TABLE 9

| | | VINA Binding |
|---|---|---|
| VINA | Compound | Energy |
| Ranking | No. | (kcal/mol) |
| 1 | 7 | 10.056 |
| 2 | 27 | 9.174 |
| 3 | 21 | 9.056 |
| 4 | 19 | 9.007 |
| 5 | 35 | 8.951 |
| 6 | 24 | 8.903 |
| 7 | 30 | 8.72 |
| 8 | 33 | 8.71 |
| 9 | 20 | 8.575 |
| 10 | 4 | 8.541 |
| 11 | 3 | 8.533 |
| 12 | 31 | 8.487 |
| 13 | 23 | 8.477 |
| 14 | 10 | 8.413 |
| 15 | 17 | 8.389 |
| 16 | 32 | 8.375 |
| 17 | 29 | 8.228 |
| 18 | 6 | 8.116 |
| 19 | 28 | 8.113 |
| 20 | 18 | 8.083 |
| 21 | 8 | 8.076 |
| 22 | 9 | 7.983 |
| 23 | 22 | 7.968 |
| 24 | 15 | 7.967 |
| 25 | 26 | 7.913 |
| 26 | 5 | 7.893 |
| 27 | 14 | 7.89 |
| 28 | 11 | 7.883 |
| 29 | 34 | 7.869 |
| 30 | 16 | 7.81 |
| 31 | 2 | 7.662 |
| 32 | 12 | 7.609 |
| 33 | 13 | 7.599 |
| 34 | 25 | 7.564 |

What is claimed is:

1. A compound having the formula: 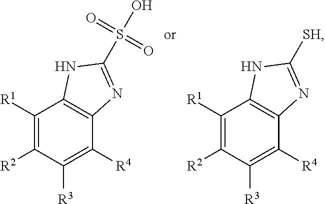
wherein R¹, R², R³, and R⁴ are independently selected from —H, halogen, —OH, —CN, —NH₂, —NO₂, —N₃, —CF₃, —C(O)O—R⁵, —S(O)ₙR⁶, —NHR⁷, —C(O)R⁸, C1-C6 alkyl, —(C1-C6 alkyl)-surface anchoring moiety, surface anchoring moiety, C2-C6 alkenyl, C2-C6 alkynyl, C1-C6 haloalkyl, C1-C6 polyhaloalkyl, C1-C6 alkoxy, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, and Cy¹; R¹ and R² are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; R² and R³ are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; R³ and R⁴ are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; wherein each n is an integer independently selected from 0, 1 and 2; wherein each R⁹, when present, is independently selected from hydrogen and C1-C8 alkyl; wherein each R¹⁰, when present, is independently selected from hydrogen and C1-C8 alkyl; wherein each R¹¹, when present, is independently selected from hydrogen and C1-C8 alkyl; wherein each R¹², when present, is independently selected from hydrogen, —OH, C1-C8 alkyl, C1-C8 hydroxyalkyl, C1-C8 monohaloalkyl, C1-C8 polyhaloalkyl, C3-C9 cycloalkyl, C2-C7 heterocycloalkyl, —(C1-C6)-Ar², and Ar²; wherein each R¹³, when present, is independently selected from hydrogen and C1-C8 alkyl; wherein each R¹⁴, when present, is independently selected from hydrogen, —OH, C1-C8 alkyl, C3-C9 cycloalkyl, C2-C7 heterocycloalkyl, C1-C8 monohaloalkyl, C1-C8 polyhaloalkyl, —(C1-C6)-Ar³, and Ar³; wherein each Ar³, when present, is independently selected from phenyl, naphthyl, and heteroaryl, and wherein each Ar³ is independently substituted with 0, 1, 2, or 3 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino; wherein each Ar², when present, is independently selected from phenyl, naphthyl, and heteroaryl, and wherein each Ar² is independently substituted with 0, 1, 2, or 3 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino; wherein each Ar¹, when present, is selected from phenyl and monocycle heteroaryl; and wherein Ar¹ is substituted with 0, 1, 2, or 3 groups independently selected from halogen, —OH, —CN, —NH₂, —NO₂, —N₃, —NHR⁷, —C(O)R⁸, —C(O)O—R⁵, —SO₂R⁶, C1-C6 alkyl, —(C1-C6 alkyl)-surface anchoring moiety, surface anchoring moiety, C1-C6 alkoxy, C1-C6 haloalkyl, or C1-C6 polyhaloalkyl, C1-C6 alkylamino, and C1-C6 dialkylamino; wherein each Cy¹, when present, is selected from C3-C9 cycloalkyl and C3-C8 heterocycloalkyl; and wherein Cy¹ is substituted with 0, 1, 2, or 3 groups independently selected from halogen, —OH, —CN, —NH₂, —NO₂, —N₃, —NHR⁷, —C(O)R⁸, —C(O)O—R⁵, —SO₂R⁶, C1-C6 alkyl, C1-C6 alkoxy, C1-C6 haloalkyl, or C1-C6 polyhaloalkyl, C1-C6 alkylamino, and C1-C6 dialkylamino; wherein each R⁵, when present, is independently selected from —H, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety; wherein each R⁶, when present, is independently selected from —H, —OH, —NHR⁷, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, and a surface anchoring moiety; wherein each R⁷, when present, is independently selected from —H, —C(O)R⁸, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety; and wherein each R⁸, when present, is independently selected from —H, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹ C1-C6-surface anchoring moiety, and a surface anchoring unit; or a pharmaceutically acceptable salt, solvate, or polymorph thereof, wherein the compound is not:

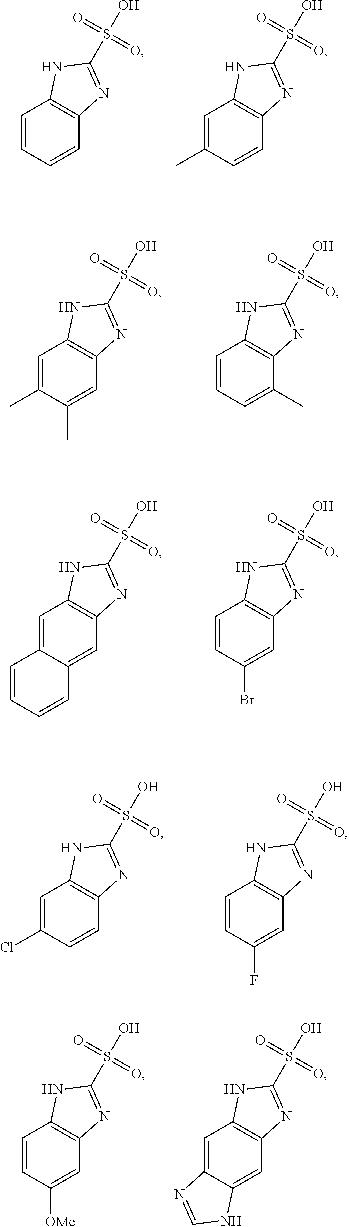

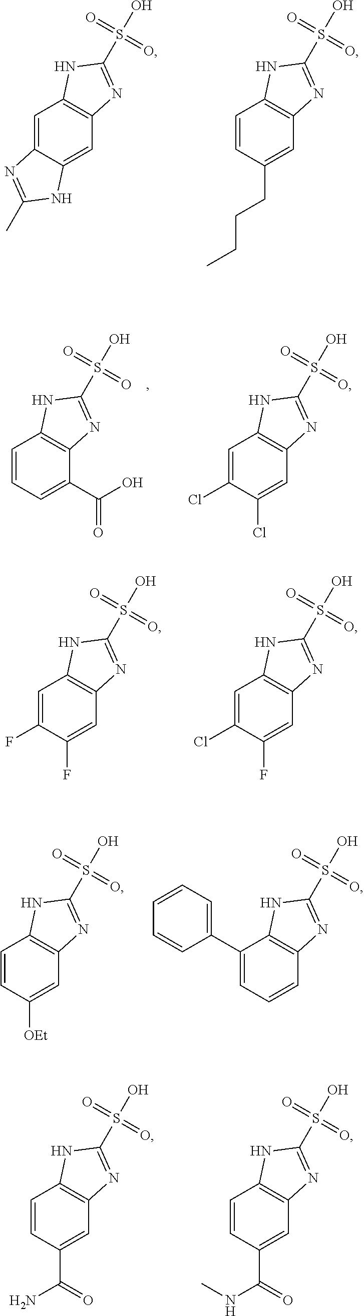

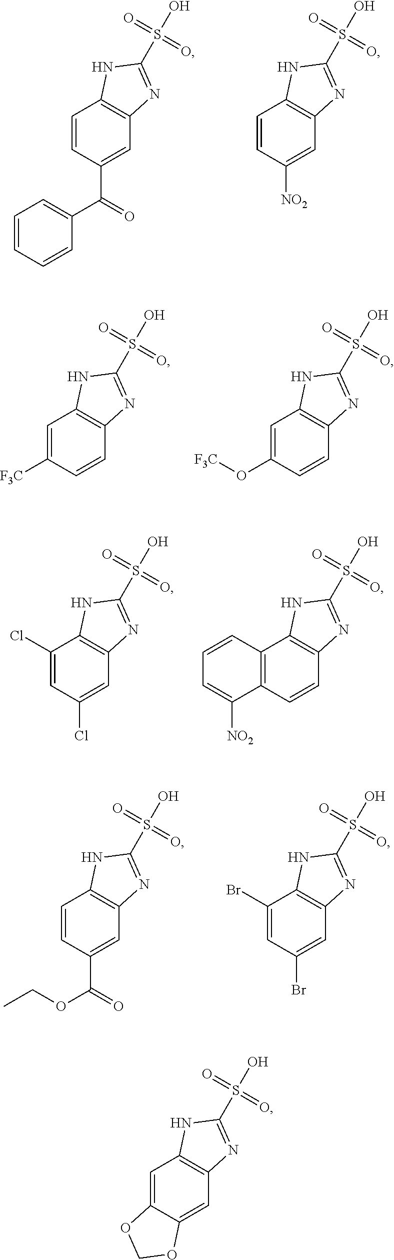

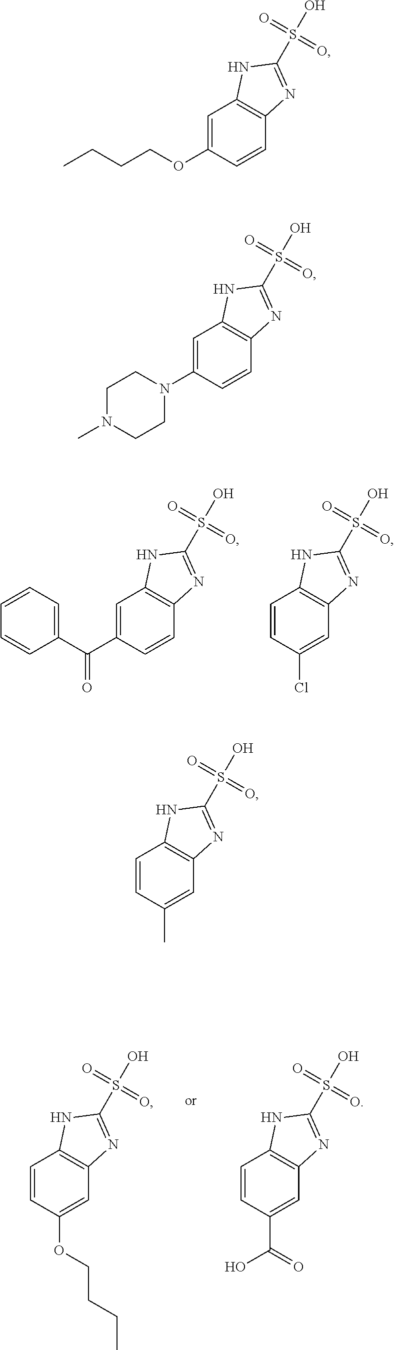

2. The compound of claim 1, wherein at least one of R¹, R², R³, and R⁴ is selected from halogen, —OH, —C(O)O—R⁵, or —C(O)R⁸.

3. The compound of claim 1, wherein at least one of R¹, R², R³, and R⁴ is a halogen.

4. The compound of claim 1, wherein R⁴ is selected from halogen, —OH, —C(O)O—R⁵, or —C(O)R⁸ and R² is a halogen.

5. The compound of claim 1, wherein at least one of R¹, R², R³, and R⁴ is S(O)ₙR⁶, wherein n is 1 or 2.

6. The compound of claim 1, wherein R² and R³ are covalently bonded together.

7. The compound of any of claim 1, wherein the compound is selected from:

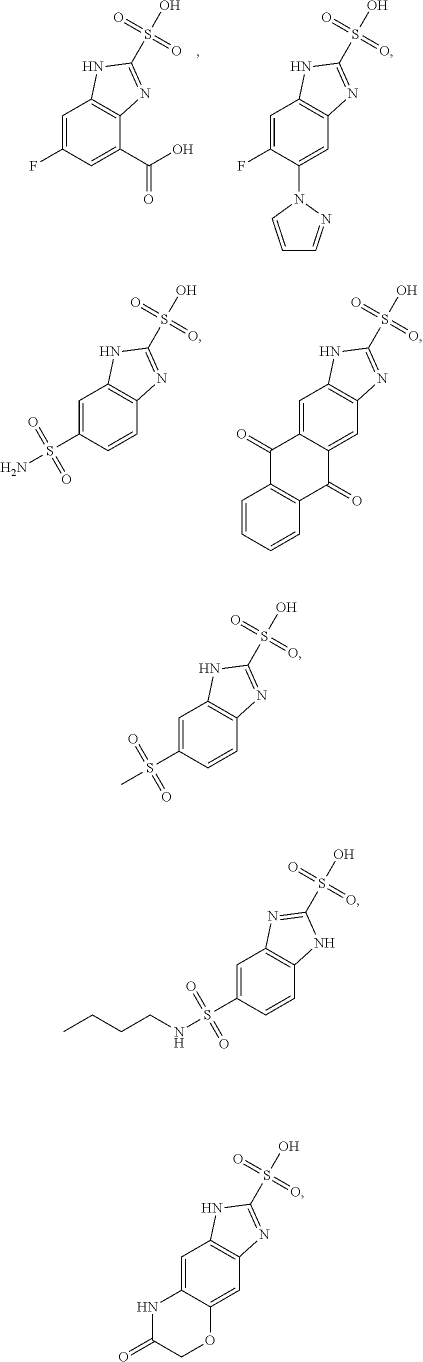

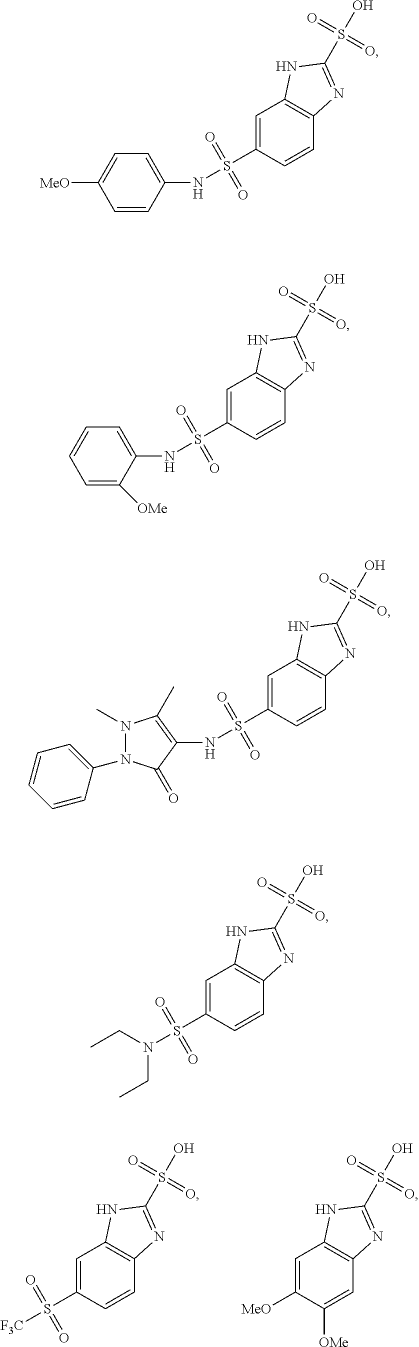 [SMILES: COc1ccc(NS(=O)(=O)c2ccc3nc(S(=O)(=O)O)[nH]c3c2)cc1 ; COc1ccccc1NS(=O)(=O)c1ccc2nc(S(=O)(=O)O)[nH]c2c1 ; Cc1c(NS(=O)(=O)c2ccc3nc(S(=O)(=O)O)[nH]c3c2)c(=O)n(-c2ccccc2)n1C ; CCN(CC)S(=O)(=O)c1ccc2nc(S(=O)(=O)O)[nH]c2c1 ; O=S(=O)(O)c1nc2ccc(S(=O)(=O)C(F)(F)F)cc2[nH]1 ; COc1cc2nc(S(=O)(=O)O)[nH]c2cc1OC]

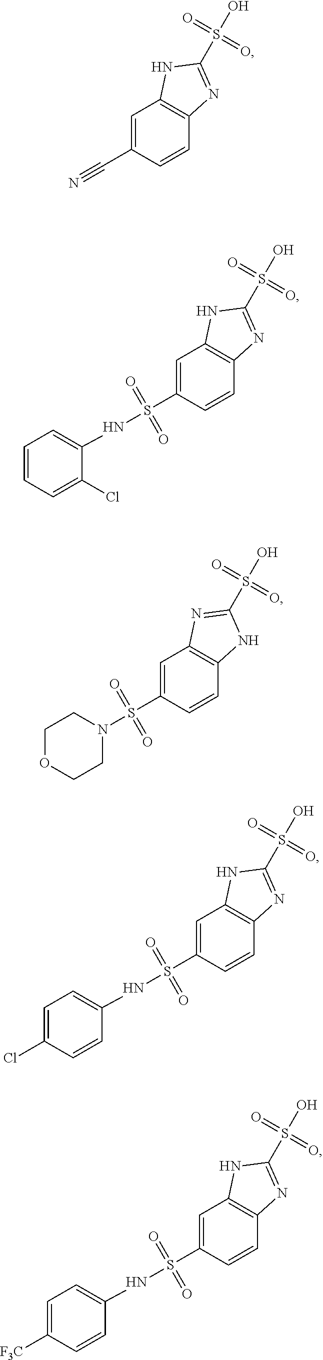 [SMILES: N#Cc1ccc2nc(S(=O)(=O)O)[nH]c2c1 ; O=S(=O)(O)c1nc2ccc(S(=O)(=O)Nc3ccccc3Cl)cc2[nH]1 ; O=S(=O)(O)c1nc2cc(S(=O)(=O)N3CCOCC3)ccc2[nH]1 ; O=S(=O)(O)c1nc2ccc(S(=O)(=O)Nc3ccc(Cl)cc3)cc2[nH]1 ; O=S(=O)(O)c1nc2ccc(S(=O)(=O)Nc3ccc(C(F)(F)F)cc3)cc2[nH]1]

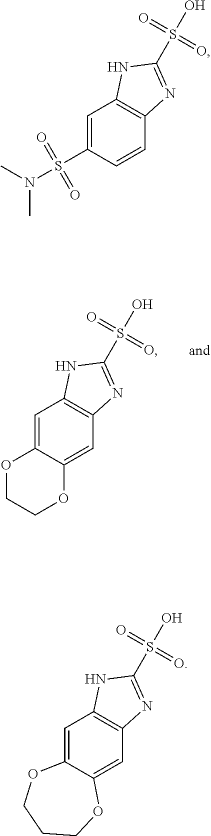 [SMILES: CN(C)S(=O)(=O)c1ccc2nc(S(=O)(=O)O)[nH]c2c1 ; O=S(=O)(O)c1nc2cc3c(cc2[nH]1)OCCO3 ; "and" ; O=S(=O)(O)c1nc2cc3c(cc2[nH]1)OCCCO3]

8. A pharmaceutical composition comprising a therapeutically effective amount of one or more compounds having the structure: 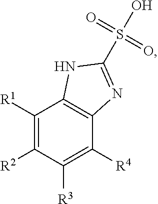
wherein R¹, R², R³, and R⁴ are independently selected from —H, halogen, —OH, —CN, —NH₂, —NO₂, —N₃, —CF₃, —C(O)O—R⁵, —S(O)ₙR⁶, —NHR⁷, —C(O)R⁸, C1-C6 alkyl, —(C1-C6 alkyl)-surface anchoring moiety, surface anchoring moiety, C2-C6 alkenyl, C2-C6 alkynyl, C1-C6 haloalkyl, C1-C6 polyhaloalkyl, C1-C6 alkoxy, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, and Cy¹; R¹ and R² are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; R² and R³ are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; R³ and R⁴ are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; wherein each n is an integer independently selected from 0, 1 and 2; wherein each R⁹, when present, is independently selected from hydrogen and C1-C8 alkyl; wherein each R¹⁰, when present, is independently selected from hydrogen and C1-C8 alkyl; wherein each R¹¹, when present, is independently selected from hydrogen and C1-C8 alkyl; wherein each R¹², when present, is independently selected from hydrogen, C1-C8 alkyl, C1-C8 hydroxyalkyl, C1-C8 monohaloalkyl, C1-C8 polyhaloalkyl, C3-C9 cycloalkyl, C2-C7 heterocycloalkyl, —(C1-C6)-Ar², and Ar²; wherein each R¹³, when present, is independently selected from hydrogen and C1-C8 alkyl; wherein each R¹⁴, when present, is independently selected from hydrogen, C1-C8 alkyl, C3-C9 cycloalkyl, C2-C7 heterocycloalkyl, C1-C8 monohaloalkyl, C1-C8 polyhaloalkyl, —(C1-C6)-Ar³, and Ar³; wherein each Ar³, when present, is independently selected from phenyl, naphthyl, and heteroaryl, and wherein each Ar³ is independently substituted with 0, 1, 2, or 3 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino; wherein each Ar², when present, is independently selected from phenyl, naphthyl, and heteroaryl, and wherein each Ar² is independently substituted with 0, 1, 2, or 3 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino; wherein each Ar¹, when present, is selected from phenyl and monocycle heteroaryl; and wherein Ar¹ is substituted with 0, 1, 2, or 3 groups independently selected from halogen, —OH, —CN, —NH₂, —NO₂, —N₃, —NHR⁷, —C(O)R⁸, —C(O)O—R⁵, —SO₂R⁶, C1-C6 alkyl, —(C1-C6 alkyl)-surface anchoring moiety, surface anchoring moiety, C1-C6 alkoxy, C1-C6 haloalkyl, or C1-C6 polyhaloalkyl, C1-C6 alkylamino, and C1-C6 dialkylamino; wherein each Cy¹, when present, is selected from C3-C9 cycloalkyl and C3-C8 heterocycloalkyl; and wherein Cy¹ is substituted with 0, 1, 2, or 3 groups independently selected from halogen, —OH, —CN, —NH₂, —NO₂, —N₃, —NHR⁷, —C(O)R⁸, —C(O)O—R⁵, —SO₂R⁶, C1-C6 alkyl, C1-C6 alkoxy, C1-C6 haloalkyl, or C1-C6 polyhaloalkyl, C1-C6 alkylamino, and C1-C6 dialkylamino; wherein each R⁵, when present, is independently selected from —H, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety; wherein each R⁶, when present, is independently selected from —H, —NHR⁷, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁶R¹⁶, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁶R¹⁶, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, and a surface anchoring moiety; wherein each R⁷, when present, is independently selected from —H, —C(O)R⁸, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁶R¹⁶, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety; and wherein each R⁸, when present, is independently selected from —H, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁶R¹⁶, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁶R¹⁶, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹ C1-C6-surface anchoring moiety, and a surface anchoring unit; or pharmaceutically acceptable salt, hydrate, solvate, or polymorph thereof, and a pharmaceutically acceptable carrier.

9. The pharmaceutical composition of claim 8, wherein the composition further comprises a penicillin, a cephalosporin, a vanomycin, a bacitracin, a monobactams, a fosfomycin, a cycloserine, or a polymyxin, or a mixture thereof.

10. A method comprising administering a therapeutically effective amount of one or more compounds to a subject, wherein the compound has the structure: 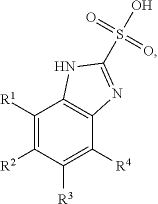
wherein R¹, R², R³, and R⁴ are independently selected from —H, halogen, —OH, —CN, —NH₂, —NO₂, —N₃, —CF₃, —C(O)O—R⁵, —S(O)ₙR⁶, —NHR⁷, —C(O)R⁸, C1-C6 alkyl, —(C1-C6 alkyl)-surface anchoring moiety, surface anchoring moiety, C2-C6 alkenyl, C2-C6 alkynyl, C1-C6 haloalkyl, C1-C6 polyhaloalkyl, C1-C6 alkoxy, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, and Cy¹; R¹ and R² are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; R² and R³ are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; R³ and R⁴ are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; wherein each n is an integer independently selected from 0, 1 and 2; wherein each R⁹, when present, is independently selected from hydrogen and C1-C8 alkyl; wherein each R¹⁰, when present, is independently selected from hydrogen and C1-C8 alkyl; wherein each R¹¹, when present, is independently selected from hydrogen and C1-C8 alkyl; wherein each R¹², when present, is independently selected from hydrogen, C1-C8 alkyl, C1-C8 hydroxyalkyl, C1-C8 monohaloalkyl, C1-C8 polyhaloalkyl, C3-C9 cycloalkyl, C2-C7 heterocycloalkyl, —(C1-C6)-Ar², and Ar²; wherein each R¹³, when present, is independently selected from hydrogen and C1-C8 alkyl; wherein each R¹⁴, when present, is independently selected from hydrogen, C1-C8 alkyl, C3-C9 cycloalkyl, C2-C7 heterocycloalkyl, C1-C8 monohaloalkyl, C1-C8 polyhaloalkyl, —(C1-C6)-Ar³, and Ar³; wherein each Ar³, when present, is independently selected from phenyl, naphthyl, and heteroaryl, and wherein each Ar³ is independently substituted with 0, 1, 2, or 3 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino; wherein each Ar², when present, is independently selected from phenyl, naphthyl, and heteroaryl, and wherein each Ar² is independently substituted with 0, 1, 2, or 3 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino; wherein each Ar¹, when present, is selected from phenyl and monocycle heteroaryl; and wherein Ar¹ is substituted with 0, 1, 2, or 3 groups independently selected from halogen, —OH, —CN, —NH₂, —NO₂, —N₃, —NHR⁷, —C(O)R⁸, —C(O)O—R⁵, —SO₂R⁶, C1-C6 alkyl, —(C1-C6 alkyl)-surface anchoring moiety, surface anchoring moiety, C1-C6 alkoxy, C1-C6 haloalkyl, or C1-C6 polyhaloalkyl, C1-C6 alkylamino, and C1-C6 dialkylamino; wherein each Cy¹, when present, is selected from C3-C9 cycloalkyl and C3-C8 heterocycloalkyl; and wherein Cy¹ is substituted with 0, 1, 2, or 3 groups independently selected from halogen, —OH, —CN, —NH₂, —NO₂, N₃, —NHR⁷, —C(O)R⁸, —C(O)O—R⁵, —SO₂R⁶, C1-C6 alkyl, C1-C6 alkoxy, C1-C6 haloalkyl, or C1-C6 polyhaloalkyl, C1-C6 alkylamino, and C1-C6 dialkylamino; wherein each R⁵, when present, is independently selected from —H, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁶, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety; wherein each R⁶, when present, is independently selected from —H, —NHR⁷, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁶, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁶, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, and a surface anchoring moiety; wherein each R⁷, when present, is independently selected from —H, —C(O)R⁸, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁶, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety; and wherein each R⁸, when present, is independently selected from H, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, (C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹ C1-C6-surface anchoring moiety, and a surface anchoring unit; or a pharmaceutically acceptable salt, solvate, or polymorph thereof.

11. The method of claim 10, wherein the composition further comprises a penicillin, a cephalosporin, a vanomycin, a bacitracin, a monobactams, a fosfomycin, a cycloserine, or a polymyxin, or a mixture thereof.

12. The method of claim 10, wherein the method further comprises administering a therapeutically effective amount of a penicillin, a cephalosporin, a vanomycin, a bacitracin, a monobactams, a fosfomycin, a cycloserine, or a polymyxin, or a mixture thereof.

13. The method of claim 10, wherein the method treats or prevents an infection in the subject.

14. The method of claim 13, wherein the infection is microbial infection.

15. The method of claim 10, wherein the method kills or inhibits the growth of microbes.

16. The method of claim 15, wherein the microbes are present internally in the subject.

17. The method of claim 15, wherein the microbes comprise bacteria.

18. The method of claim 17, wherein the bacteria comprises Bacillus anthracis, Staphylococcus aureus, Bacillus subtilis, or Helicobacter pylori, or a mixture thereof.

19. An article comprising at least a first surface, wherein the first surface comprises one or more compounds having the formula: 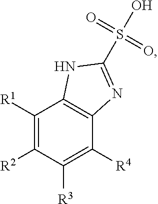
wherein R¹, R², R³, and R⁴ are independently selected from —H, halogen, —OH, —CN, —NH₂, —NO₂, —N₃, —CF₃, —C(O)O—R⁵, —S(O)ₙR⁶, —NHR⁷, —C(O)R⁸, C1-C6 alkyl, —(C1-C6 alkyl)-surface anchoring moiety, surface anchoring moiety, C2-C6 alkenyl, C2-C6 alkynyl, C1-C6 haloalkyl, C1-C6 polyhaloalkyl, C1-C6 alkoxy, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, and Cy¹; R¹ and R² are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; R² and R³ are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; R³ and R⁴ are optionally covalently bonded together to form a 3- to 10-membered monocycle or multicycle substituted with 0, 1, 2, or 3 groups independently selected from halogen, —NH₂, —OH, —CN, C1-C6 alkyl, C1-C6 haloalkyl, C1-C6 polyhaloalkyl, C1-C6 alkoxy, C1-C6 alkylamino, C1-C6 dialkylamino, Ar¹, and Cy¹; wherein each n is an integer independently selected from 0, 1 and 2; wherein each R⁹, when present, is independently selected from hydrogen and C1-C8 alkyl; wherein each R¹⁰, when present, is independently selected from hydrogen and C1-C8 alkyl; wherein each R¹¹, when present, is independently selected from hydrogen and C1-C8 alkyl; wherein each R¹², when present, is independently selected from hydrogen, C1-C8 alkyl, C1-C8 hydroxyalkyl, C1-C8 monohaloalkyl, C1-C8 polyhaloalkyl, C3-C9 cycloalkyl, C2-C7 heterocycloalkyl, —(C1-C6)-Ar², and Ar²; wherein each R¹³, when present, is independently selected from hydrogen and C1-C8 alkyl; wherein each R¹⁴, when present, is independently selected from hydrogen, C1-C8 alkyl, C3-C9 cycloalkyl, C2-C7 heterocycloalkyl, C1-C8 monohaloalkyl, C1-C8 polyhaloalkyl, —(C1-C6)-Ar³, and Ar³; wherein each Ar³, when present, is independently selected from phenyl, naphthyl, and heteroaryl, and wherein each Ar³ is independently substituted with 0, 1, 2, or 3 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino; wherein each Ar², when present, is independently selected from phenyl, naphthyl, and heteroaryl, and wherein each Ar² is independently substituted with 0, 1, 2, or 3 groups independently selected from halogen, —NH₂, —OH, —CN, C1-C8 alkyl, —(C1-C6 alkyl)-surface anchoring moiety, surface anchoring moiety, C1-C8 monohaloalkyl, C1-C8 polyhaloalkyl, C1-C8 alkoxy, C1-C8 alkylamino, and C1-C8 dialkylamino; wherein each Ar¹, when present, is selected from phenyl and monocycle heteroaryl; and wherein Ar¹ is substituted with 0, 1, 2, or 3 groups independently selected from halogen, —OH, —CN, —NH₂, —NO₂, —N₃, —NHR⁷, —C(O)R⁸, —C(O)O—R⁵, —SO₂R⁶, C1-C6 alkyl, —(C1-C6 alkyl)-surface anchoring moiety, surface anchoring moiety, C1-C6 alkoxy, C1-C6 haloalkyl, or C1-C6 polyhaloalkyl, C1-C6 alkylamino, and C1-C6 dialkylamino; wherein each Cy¹, when present, is selected from C3-C9 cycloalkyl and C3-C8 heterocycloalkyl; and wherein Cy¹ is substituted with 0, 1, 2, or 3 groups independently selected from halogen, —OH, —CN, —NH₂, —NO₂, N₃, —NHR⁷, —C(O)R⁸, —C(O)O—R⁵, —SO₂R⁶, C1-C6 alkyl, C1-C6 alkoxy, C1-C6 haloalkyl, or C1-C6 polyhaloalkyl, C1-C6 alkylamino, and C1-C6 dialkylamino; wherein each R⁵, when present, is independently selected from —H, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety; wherein each R⁶, when present, is independently selected from —H, —NHR⁷, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², —NR¹¹(C1-C6 alkyl)-S(O)ₙR¹², —NR¹¹(C1-C6 alkyl)-S(O)ₙNR⁹R¹⁰, —NR¹¹(C═O)R¹², —NR¹¹(C═O)OR¹², —NR¹¹(C═O)NR¹², —NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, and a surface anchoring moiety; wherein each R⁷, when present, is independently selected from —H, —C(O)R⁸, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —(C1-C6 alkyl)-(C═O)R¹², —(C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, —S(O)ₙNR¹³R¹⁴, —(C1-C3 alkyl)-Ar¹, Ar¹, —(C1-C3 alkyl)-Cy¹, Cy¹, C1-C6-surface anchoring moiety, and a surface anchoring moiety; and wherein each R⁸, when present, is independently selected from —H, C1-C6 alkyl, C2-C6 alkenyl, C2-C6 alkynyl, C1-C6 haloalkyl, C1-C6 polyhaloalkyl, C1-C6 alkylamino, C1-C6 dialkylamino, —(C1-C6 alkyl)-NR⁹R¹⁰, —(C1-C6 alkyl)-NR¹¹(C═O)R¹², —(C1-C6 alkyl)-NR¹¹(C═O)OR¹², —(C1-C6 alkyl)-NR¹¹(C═O)NR¹², —(C1-C6 alkyl)-NR¹¹S(O)ₙR¹², —NR¹¹(C1-C6 alkyl)-(C═O)R¹², —NR¹¹(C1-C6 alkyl)-(C═O)OR¹², —NR¹¹(C1-C6 alkyl)-(C═O)NR¹², NR¹¹(C1-C6 alkyl)-S(O)ₙR¹², —NR¹¹(C1-C6 alkyl)-S(O)ₙNR⁹R¹⁰, —NR¹¹(C═O)R¹², NR¹¹(C═O)OR¹², —NR¹¹(C═O)NR¹², —NR¹¹S(O)ₙR¹², —(C1-C6 alkyl)-(C═O)R¹², (C1-C6 alkyl)-(C═O)OR¹², —(C1-C6 alkyl)-(C═O)NR¹², —(C1-C6 alkyl)-S(O)ₙR¹², —(C1-C6 alkyl)-S(O)ₙNR¹³R¹⁴, S(O)ₙNR¹³R¹⁴, —(C1-C3 alkyl)-Ar¹, Ar¹, —(C1-C3 alkyl)-Cy¹, Cy¹ C1-C6-surface anchoring moiety, and a surface anchoring unit; or a pharmaceutically acceptable salt, solvate, or polymorph thereof.

20. The article of claim 19, wherein the compound is covalently bonded to the first surface via a surface anchoring moiety.

21-23. (canceled)
